(19) United States
(12) Patent Application Publication
(10) Pub. No.: US 2026/0092076 A1
(43) Pub. Date: Apr. 2, 2026

(54) NOVEL COMPOUNDS

(71) Applicant: Hoffmann-La Roche Inc., Little Falls, NJ (US)

(72) Inventors: Lewis Scott AITKEN, Nottingham (GB); Lea Aurelie BOUCHE, Basel (CH); Wolfgang GUBA, Muellheim (DE); Georg Stefan JAESCHKE, Basel (CH); Stefanie Katharina MESCH, Basel (CH); Christian SCHNIDER, Biel-Benken (CH); Jonathan Martin SHANNON, Nottingham (GB); Sandra STEINER, Sursee (CH)

(73) Assignee: Hoffmann-La Roche Inc., Little Falls, NJ (US)

(21) Appl. No.: 19/351,608

(22) Filed: Oct. 7, 2025

Related U.S. Application Data
(63) Continuation of application No. PCT/EP2024/059641, filed on Apr. 10, 2024.

(30) Foreign Application Priority Data
Apr. 12, 2023 (EP) 23167556.2

(51) Int. Cl. C07D 519/00; A61K 31/437; A61K 31/4375; A61K 31/4725; A61K 31/4985; A61K 31/5025; A61K 31/519; C07D 498/04

(52) U.S. Cl. CPC C07D 519/00; A61K 31/437; A61K 31/4375; A61K 31/4725; A61K 31/4985; A61K 31/5025; A61K 31/519; C07D 498/04

(57) ABSTRACT

The invention relates to novel compounds having the general formula I

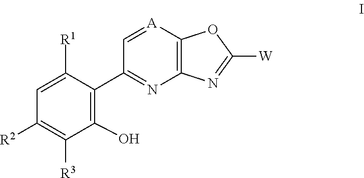

wherein R¹, R², R³, A and W are as described herein, composition including the compounds and methods of using the compounds.

### FIELD OF THE INVENTION

The present invention relates to organic compounds useful for therapy and/or prophylaxis in a mammal, and in particular to compounds that modulate NLRP3 inhibition.

The present invention provides novel compounds of formula I

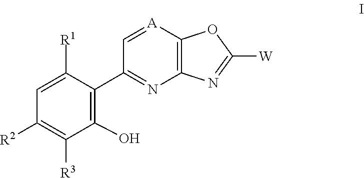

wherein,
R¹ is H, alkyl, hydroxyalkyl or alkoxyalkyl;
R² is halo, haloalkyl or cyano;
R³ is H;
or R² and R³, and the atoms to which they are bonded, form either a 4-6 membered heterocycle ring comprising a single O heteroatom optionally substituted with one or two substituents independently selected from halo and alkyl, or R² and R³, and the atoms to which they are bonded, form a 3-6 membered cycloalkyl ring optionally substituted with one or two substituents independently selected from halo and alkyl;
A is CH or N;
W is:

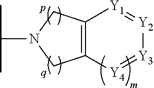

p is 1 or 2;
q is 1 or 2;
m is 0 or 1;
Y₁ is C═O, CH, CH₂, C—R^{x}, N, or NH
Y₂ is C═O, CH, C—R^{y}, O, N, NH, or N—CH₃,
Y₃ is C═O, CH, CH₂, CR^{y}, N, NH, or N^{z}
Y₄ is C═O, CH, CR^{y}, N, NH or N—CH₃
wherein R^{x} is H and each R^{y} is independently selected from —OH, alkyl, alkoxy, cyano and halo, or R^{x}+R^{y}, through the atoms to which they are attached, form a heterocycle ring;
R^{z} is H, alkyl or hydroxyalkyl;
and pharmaceutically acceptable salts thereof.

Furthermore, the invention includes all racemic mixtures, all their corresponding enantiomers and/or optical isomers.

### BACKGROUND OF THE INVENTION

The NOD-like receptor (NLR) family, pyrin domain-containing protein 3 (NLRP3) inflammasome is a component of the inflammatory process, and its aberrant activity is pathogenic in inherited disorders such as cryopyrin-associated periodic syndromes (CAPS) and complex diseases such as multiple sclerosis, type 2 diabetes, Alzheimer's disease and atherosclerosis.

NLRP3 is an intracellular signaling molecule that senses many pathogen-derived, environmental and host-derived factors. Upon activation, NLRP3 binds to apoptosis-associated speck-like protein containing a caspase activation and recruitment domain (ASC). ASC then polymerises to form a large aggregate known as an ASC speck. Polymerised ASC in turn interacts with the cysteine protease caspase-1 to form a complex termed the inflammasome. This results in the activation of caspase-1, which cleaves the precursor forms of the proinflammatory cytokines IL-1β and IL-18 (termed pro-IL-1β and pro-IL-18 respectively) to thereby activate these cytokines. Caspase-1 also mediates a type of inflammatory cell death known as pyroptosis. The ASC speck can also recruit and activate caspase-8, which can process pro-IL-1β and pro-IL-18 and trigger apoptotic cell death.

Caspase-1 cleaves pro-IL-1β and pro-IL-18 to their active forms, which are secreted from the cell. Active caspase-1 also cleaves gasdermin-D to trigger pyroptosis. Through its control of the pyroptotic cell death pathway, caspase-1 also mediates the release of alarmin molecules such as IL-33 and high mobility group box 1 protein (HMGB1). Caspase-1 also cleaves intracellular IL-IR2 resulting in its degradation and allowing the release of IL-1α. In human cells caspase-1 may also control the processing and secretion of IL-37. A number of other caspase-1 substrates such as components of the cytoskeleton and glycolysis pathway may contribute to caspase-1-dependent inflammation.

NLRP3-dependent ASC specks are released into the extracellular environment where they can activate caspase-1, induce processing of caspase-1 substrates and propagate inflammation.

Active cytokines derived from NLRP3 inflammasome activation are important drivers of inflammation and interact with other cytokine pathways to shape the immune response to infection and injury. For example, IL-1β signalling induces the secretion of the pro-inflammatory cytokines IL-6 and TNF. IL-1β and IL-18 synergise with IL-23 to induce IL-17 production by memory CD4 Th17 cells and by γδ T cells in the absence of T cell receptor engagement. IL-18 and IL-12 also synergise to induce IFN-γ production from memory T cells and NK cells driving a Th1 response.

The inherited CAPS diseases Muckle-Wells syndrome (MWS), familial cold autoinflammatory syndrome (FCAS) and neonatal-onset multisystem inflammatory disease (NOMID) are caused by gain-of-function mutations in NLRP3, thus defining NLRP3 as a critical component of the inflammatory process. NLRP3 has also been implicated in the pathogenesis of a number of complex diseases, notably including metabolic disorders such as type 2 diabetes, atherosclerosis, obesity and gout.

A role for NLRP3 in diseases of the central nervous system is emerging, and lung diseases have also been shown to be influenced by NLRP3. NLRP3 has also been suggested to have a role in a number of central nervous system conditions, including Parkinson's disease (PD), Alzheimer's disease (AD), dementia, Huntington's disease, cerebral malaria, brain injury from pneumococcal meningitis (Walsh et al., Nature Reviews, 15: 84-97, 2014, and Dempsey et al. Brain. Behav. Immun. 201761: 306-316). NLRP3 has also been shown to play a role in a number of lung diseases including chronic obstructive pulmonary disorder (COPD), asthma (including steroid-resistant asthma), asbestosis, and silicosis (De Nardo et al., Am. J. Pathol., 184: 42-54, 2014 and Kim et al. Am J Respir Crit Care Med. 2017 196(3): 283-97). Furthermore, NLRP3 has a role in the development of liver disease, kidney disease and aging. Many of these associations were defined using Nlrp3^{-/-} mice, but there have also been insights into the specific activation of NLRP3 in these diseases. In type 2 diabetes mellitus (T2D), the deposition of islet amyloid polypeptide in the pancreas activates NLRP3 and IL-1β signalling, resulting in cell death and inflammation.

Several small molecules have been shown to inhibit the NLRP3 inflammasome. Glyburide inhibits IL-1β production at micromolar concentrations in response to the activation of NLRP3 but not NLRC4 or NLRP1. Other previously characterised weak NLRP3 inhibitors include parthenolide, 3,4-methylenedioxy-β-nitrostyrene and dimethyl sulfoxide (DMSO), although these agents have limited potency and are nonspecific.

Current treatments for NLRP3-related diseases include biologic agents that target IL-1. These are the recombinant IL-1 receptor antagonist anakinra, the neutralizing IL-1β antibody canakinumab and the soluble decoy IL-1 receptor rilonacept. These approaches have proven successful in the treatment of CAPS, and these biologic agents have been used in clinical trials for other IL-1β-associated diseases.

There is a need to provide compounds with improved pharmacological and/or physiological and/or physicochemical properties and/or those that provide a useful alternative to known compounds.

### SUMMARY OF THE INVENTION

The present invention provides novel compounds of formula I

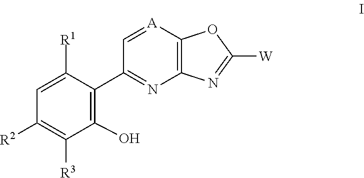

wherein,
R¹ is H, alkyl, hydroxyalkyl or alkoxyalkyl;
R² is halo, haloalkyl or cyano;
R³ is H;
or R² and R³, and the atoms to which they are bonded, form either a 4-6 membered heterocycle ring comprising a single O heteroatom optionally substituted with one or two substituents independently selected from halo and alkyl, or R² and R³, and the atoms to which they are bonded, form a 3-6 membered cycloalkyl ring optionally substituted with one or two substituents independently selected from halo and alkyl;
A is CH or N;
W is:

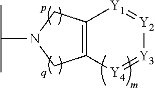

p is 1 or 2;
q is 1 or 2;
m is 0 or 1;
Y₁ is C═O, CH, CH₂, C—R^{x}, N, or NH
Y₂ is C═O, CH, C—R^{y}, O, N, NH, or N—CH₃,
Y₃ is C═O, CH, CH₂, CR^{y}, N, NH, or NR^{z}
Y₄ is C═O, CH, CR^{y}, N, NH or N—CH₃
wherein R^{x} is H and each R^{y} is independently selected from —OH, alkyl, alkoxy, cyano and halo, or R^{x}+R^{y}, through the atoms to which they are attached, form a heterocycle ring;
R^{z} is H, alkyl or hydroxyalkyl;
and pharmaceutically acceptable salts thereof.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl group is methyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy. Particular alkoxy group is methoxy.

The term “alkoxyalkyl” denotes an alkyl group wherein one of the hydrogen atoms of the alkyl group have been replaced by an alkoxy group. Examples of alkoxyalkyl are methoxymethyl and methoxyethyl.

The term “cycloalkyl” denotes monocyclic or polycyclic saturated or partially unsaturated, non-aromatic hydrocarbon. In some embodiments, unless otherwise described, cycloalkyl comprises 3 to 8 carbon atoms, 3 to 6 carbon atoms, or 3 to 5 carbon atoms. In some embodiments, cycloalkyl is a saturated monocyclic or polycyclic hydrocarbon. Examples of cycloalkyl groups include cyclopropyl, cyclobutyl, cyclopentyl and cyclohexyl, and the like.

The term “halogen”, “halide” and “halo” are used interchangeably herein and denote fluoro, chloro, bromo or iodo. Particular examples of halo are fluoro and chloro.

The term “haloalkyl” denotes a C₁₋₆-alkyl group wherein at least one of the hydrogen atoms of the C₁₋₆-alkyl group has been replaced by the same or different halogen atoms. Examples of haloalkyl include fluoromethyl, difluoromethyl and trifluoromethyl. Particular examples of haloalkyl include trifluoromethyl and difluoromethyl.

The term “heterocycle ring” denotes a monovalent saturated or partly unsaturated mono- or bicyclic ring system of 4 to 9 ring atoms, comprising 1, 2, or 3 ring heteroatoms selected from N, O and S, the remaining ring atoms being carbon. Examples for monocyclic saturated heterocycle rings are azetidinyl, diazepanyl, pyrrolidinyl, tetrahydrofuranyl, pyrazolidinyl, imidazolidinyl, oxazolidinyl, isoxazolidinyl, thiazolidinyl, piperidinyl, tetrahydropyranyl, tetrahydrothiopyranyl, morpholinyl, and piperazinyl. Examples of polycyclic saturated heterocycle rings are azaspiroheptanyl, diazaspiroheptanyl, azaspirooctanyl, diazospirooctanyl, diazaspirononanyl, oxaazaspirooctanyl, and oxadiazaspirononanyl.

The term “hydroxy” denotes a —OH group.

The term “hydroxyalkyl” denotes an alkyl group wherein at least one of the hydrogen atoms of the alkyl group has been replaced by a hydroxy group. Examples of hydroxyalkyl include hydroxy methyl, hydroxyethyl, hydroxypropyl, hydroxymethylethyl, hydroxy methylpropyl and dihydroxypropyl. Particular examples of hydroxyalkyl include hydroxymethyl and hydroxyethyl.

The term “cyano” denotes a —C≡N group.

The term “pharmaceutically acceptable salts” refers to those salts which retain the biological effectiveness and properties of the free bases or free acids, which are not biologically or otherwise undesirable. The salts are formed with inorganic acids such as trifluoroacetic acid, hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein R¹ is alkyl or hydroxyalkyl.

An embodiment of the present invention provides compounds according to formula I as described herein, wherein R¹ is alkyl.

An embodiment of the present invention provides compounds according to formula I as described herein, wherein n is 1.

An embodiment of the present invention provides compounds according to formula I as described herein, wherein q is 1.

An embodiment of the present invention provides compounds according to formula I as described herein, wherein m is 1.

An embodiment of the present invention provides compounds according to formula I as described herein, wherein Y₁ is CH, C—R^{x}, N, or NH.

An embodiment of the present invention provides compounds according to formula I as described herein, wherein Y₁ is CH, N, or NH.

An embodiment of the present invention provides compounds according to formula I as described herein, wherein Y₁ is CH.

An embodiment of the present invention provides compounds according to formula I as described herein, wherein Y₂ is C═O, CH, C—R^{y}, N, or NH.

An embodiment of the present invention provides compounds according to formula I as described herein, wherein Y₂ is C═O or N.

An embodiment of the present invention provides compounds according to formula I as described herein, wherein Y₃ is C═O, CH, C—R^{y}, N, NH or NR_{z}.

An embodiment of the present invention provides compounds according to formula I as described herein, wherein Y₃ is CH, N, NH or NR_{z}.

An embodiment of the present invention provides compounds according to formula I as described herein, wherein Y₃ is CH, NH or NR_{z}.

An embodiment of the present invention provides compounds according to formula I as described herein, wherein Y₄ is C═O, CH, CR^{y}, N, NH or N—CH₃, wherein R^{x} is H and each R^{y} is independently selected from OH, alkyl, alkoxy, cyano and halo, or R^{x}+R^{y}, through the atoms to which they are attached, form a heterocycle ring comprising 1 or 2 oxygen heteroatoms.

An embodiment of the present invention provides compounds according to formula I as described herein, wherein each R^{y} is independently selected from OH, alkyl, alkoxy, cyano and halo.

An embodiment of the present invention provides compounds according to formula I as described herein, wherein each R^{y} is alkyl.

An embodiment of the present invention provides compounds according to formula I as described herein, wherein each R^{z} is H, alkyl or hydroxyalkyl.

An embodiment of the present invention provides compounds according to formula I as described herein, wherein each R^{z} is hydroxyalkyl.

An embodiment of the present invention provides compounds according to formula I as described herein, wherein,
R¹ is alkyl or hydroxyalkyl;
R² is halo, haloalkyl, or cyano;
R³ is H;
A is CH or N;
p is 1 or 2;
q is 1 or 2;
m is 0 or 1;
Y₁ is CH, C—R^{x}, N, or NH;
Y₂ is C═O, CH, C—R^{y}, N, or NH;
Y₃ is C═O, CH, CR^{y}, N, NH or NR^{z};
Y₄ is C═O, CH, CR^{y}, N, NH or N—CH₃;
   wherein R^{x} is H and each R^{y} is independently selected from OH, alkyl, alkoxy, cyano and halo, or R^{x}+R^{y}, through the atoms to which they are attached, form a heterocycle ring comprising 1 or 2 oxygen heteroatoms;
   R^{z} is H, alkyl or hydroxyalkyl;
and pharmaceutically acceptable salts thereof.

An embodiment of the present invention provides compounds according to formula I as described herein, wherein,
R¹ is alkyl;
R² is halo, haloalkyl, or cyano;
R³ is H;
A is CH or N;
p is 1 or 2;
q is 1;
m is 0 or 1;
Y₁ is CH, N, or NH;
Y₂ is C═O, CH, C—R^{y}, N, or NH;
Y₃ is CH, N, NH or NR^{z};
Y₄ is CH or N;
   wherein each R^{y} is alkyl;
   R^{z} is H, alkyl or hydroxyalkyl;
and pharmaceutically acceptable salts thereof.

An embodiment of the present invention provides compounds according to formula I as described herein, wherein,
R¹ is alkyl;
R² is halo, haloalkyl, or cyano;
R³ is H;
A is CH or N;
p is 1 or 2;
q is 1;
m is 0 or 1;
Y₁ is CH, N, or NH;
Y₂ is C═O, CH, C—R^{y}, N, or NH;
Y₃ is CH, N, NH or NR^{z};
Y₄ is CH or N;
   wherein each R^{y} is alkyl;
   R^{z} is hydroxyalkyl.
and pharmaceutically acceptable salts thereof.

An embodiment of the present invention provides compounds according to formula I as described herein, wherein,
R¹ is alkyl;
R² is halo, haloalkyl, or cyano;
R³ is H;
A is CH or N;
p is 1;
q is 1;
m is 1;
Y₁ is CH;
Y₂ is C═O or N;
Y₃ is CH, NH or NR^{z};
Y₄ is CH;
R^{z} is hydroxyalkyl;
and pharmaceutically acceptable salts thereof.

Particular examples of compounds of formula I as described herein are selected from
2-[5-[2-Hydroxy-6-methyl-4-(trifluoromethyl)phenyl]oxazolo[4,5-b]pyridin-2-yl]-3,5-dihydro-1H-pyrrolo[3,4-c]pyridin-6-one;
2-[5-[2-Hydroxy-6-methyl-4-(trifluoromethyl)phenyl]oxazolo[4,5-b]pyridin-2-yl]-5-methyl-1,3-dihydropyrrolo[3,4-c]pyridin-6-one;
3-Hydroxy-5-methyl-4-[2-(6-oxo-3,5-dihydro-1H-pyrrolo[3,4-c]pyridin-2-yl)oxazolo[4,5-b]pyridin-5-yl]benzonitrile;
2-[5-(4-Chloro-2-hydroxy-6-methyl-phenyl)oxazolo[4,5-b]pyridin-2-yl]-3,5-dihydro-1H-pyrrolo[3,4-c]pyridin-6-one;
5-(2-Hydroxyethyl)-2-[5-[2-hydroxy-6-methyl-4-(trifluoromethyl)phenyl]oxazolo[4,5-b]pyridin-2-yl]-1,3-dihydropyrrolo[3,4-c]pyridin-6-one;
5-Chloro-2-(2-isoindolin-2-yloxazolo[4,5-b]pyridin-5-yl)-3-methyl-phenol;
5-chloro-2-[2-(1,3-dihydropyrrolo[3,4-c]pyridin-2-yl)oxazolo[4,5-b]pyridin-5-yl]-3-methyl-phenol;
2-[5-(4-Chloro-2-hydroxy-6-methyl-phenyl)oxazolo[4,5-b]pyridin-2-yl]isoindolin-5-ol;
5-Chloro-2-[2-(4-fluoroisoindolin-2-yl)oxazolo[4,5-b]pyridin-5-yl]-3-methyl-phenol;
4-[2-(1,3-dihydropyrrolo[3,4-c]pyridin-2-yl)oxazolo[4,5-b]pyridin-5-yl]-3-hydroxy-5-methyl-benzonitrile;
5-chloro-3-methyl-2-[2-(2-methyl-7,8-dihydro-5H-pyrido[4,3-d]pyrimidin-6-yl)oxazolo[4,5-b]pyridin-5-yl]phenol;
2-[2-(6-methoxy-1,3-dihydropyrrolo[3,4-c]pyridin-2-yl)oxazolo[4,5-b]pyridin-5-yl]-3-methyl-5-(trifluoromethyl)phenol;
5-chloro-2-[2-(5,7-dihydropyrrolo[3,4-d]pyrimidin-6-yl)oxazolo[4,5-b]pyridin-5-yl]-3-methyl-phenol; formic acid;
5-chloro-2-[2-(5,7-dihydropyrrolo[3,4-d]pyrimidin-6-yl)oxazolo[4,5-b]pyridin-5-yl]-3-methyl-phenol;
5-chloro-2-[2-(4,6-dihydro-1H-pyrrolo[3,4-c]pyrazol-5-yl)oxazolo[4,5-b]pyridin-5-yl]-3-methyl-phenol;
5-chloro-2-[2-(3,4-dihydro-1H-2,7-naphthyridin-2-yl)oxazolo[4,5-b]pyridin-5-yl]-3-methyl-phenol;
3-hydroxy-4-(2-isoindolin-2-yloxazolo[4,5-b]pyridin-5-yl)-5-methyl-benzonitrile;
4-[2-(7,8-dihydro-5H-pyrido[4,3-d]pyrimidin-6-yl)oxazolo[4,5-b]pyridin-5-yl]-3-hydroxy-5-methyl-benzonitrile;
2-[2-(5,7-dihydropyrrolo[3,4-d]pyrimidin-6-yl)oxazolo[4,5-b]pyridin-5-yl]-3-methyl-5-(trifluoromethyl)phenol;
7-[5-[2-hydroxy-6-methyl-4-(trifluoromethyl)phenyl]oxazolo[4,5-b]pyridin-2-yl]-2,5,6,8-tetrahydro-2,7-naphthyridin-3-one;
6-[5-[2-hydroxy-6-methyl-4-(trifluoromethyl)phenyl]oxazolo[4,5-b]pyridin-2-yl]-5,7-dihydro-1H-pyrrolo[3,4-b]pyridin-2-one;
6-[5-[2-hydroxy-6-methyl-4-(trifluoromethyl)phenyl]oxazolo[4,5-b]pyridin-2-yl]-1-methyl-5,7-dihydropyrrolo[3,4-b]pyridin-2-one;
6-[5-[2-hydroxy-6-methyl-4-(trifluoromethyl)phenyl]oxazolo[4,5-b]pyridin-2-yl]-2-methyl-5,7-dihydropyrrolo[3,4-c]pyridazin-3-one;
2-[2-(5,7-dihydropyrrolo[3,4-b]pyrazin-6-yl)oxazolo[4,5-b]pyridin-5-yl]-3-methyl-5-(trifluoromethyl)phenol;
2-[2-(7,8-dihydro-5H-pyrido[4,3-d]pyrimidin-6-yl)oxazolo[4,5-b]pyridin-5-yl]-3-methyl-5-(trifluoromethyl)phenol;
5-chloro-3-methyl-2-[2-(2,4,6,7-tetrahydropyrazolo[4,3-c]pyridin-5-yl)oxazolo[4,5-b]pyridin-5-yl]phenol;
5-(difluoromethyl)-2-[2-(7,8-dihydro-5H-pyrido[4,3-d]pyrimidin-6-yl)oxazolo[4,5-b]pyridin-5-yl]-3-methyl-phenol;
7-[5-(4-chloro-2-hydroxy-6-methyl-phenyl)oxazolo[4,5-b]pyridin-2-yl]-2,5,6,8-tetrahydro-2,7-naphthyridin-3-one;
5-Chloro-2-[2-(1,3-dihydropyrrolo[3,4-c]pyridin-2-yl)oxazolo[4,5-b]pyrazin-5-yl]-3-methyl-phenol; formic acid;
5-Chloro-2-[2-(1,3-dihydropyrrolo[3,4-c]pyridin-2-yl)oxazolo[4,5-b]pyrazin-5-yl]-3-methyl-phenol;
5-Chloro-2-[2-(1,3-dihydropyrrolo[3,4-c]pyridin-2-yl)oxazolo[4,5-b]pyrazin-5-yl]-3-(hydroxymethyl)phenol;
5-Chloro-2-[2-(7,8-dihydro-5H-pyrido[4,3-d]pyrimidin-6-yl)oxazolo[4,5-b]pyridin-5-yl]-3-methyl-phenol;
5-Chloro-2-[2-(2-methoxy-7,8-dihydro-5H-pyrido[4,3-d]pyrimidin-6-yl)oxazolo[4,5-b]pyridin-5-yl]-3-methyl-phenol;
2-[5-(4-Chloro-2-hydroxy-6-methyl-phenyl)oxazolo[4,5-b]pyridin-2-yl]-3,4-dihydro-1H-isoquinoline-5-carbonitrile;
5-Chloro-2-[2-(5-methoxy-3,4-dihydro-1H-isoquinolin-2-yl)oxazolo[4,5-b]pyridin-5-yl]-3-methyl-phenol;
5-Chloro-2-[2-(7-fluoro-3,4-dihydro-1H-isoquinolin-2-yl)oxazolo[4,5-b]pyridin-5-yl]-3-methyl-phenol;
6-[5-(4-Chloro-2-hydroxy-6-methyl-phenyl)oxazolo[4,5-b]pyridin-2-yl]-1,5,7,8-tetrahydropyrido[4,3-d]pyrimidin-2-one;
6-[5-(4-Chloro-2-hydroxy-6-methyl-phenyl)oxazolo[4,5-b]pyridin-2-yl]-1,5,7,8-tetrahydro-1,6-naphthyridin-2-one;
6-[5-(4-Chloro-2-hydroxy-6-methyl-phenyl)oxazolo[4,5-b]pyridin-2-yl]-5,7-dihydro-2H-pyrrolo[3,4-c]pyridazin-3-one;
5-chloro-2-[2-(7,8-dihydro-5H-pyrido[3,4-b]pyrazin-6-yl)oxazolo[4,5-b]pyridin-5-yl]-3-methyl-phenol;
6-[5-[2-hydroxy-6-methyl-4-(trifluoromethyl)phenyl]oxazolo[4,5-b]pyridin-2-yl]-3,5,7,8-tetrahydropyrido[4,3-d]pyrimidin-4-one;
5-chloro-2-(2-(6,8-dihydro-7H-[1,3]dioxolo[4,5-e]isoindol-7-yl)oxazolo[4,5-b]pyridin-5-yl)-3-methylphenol;
   and pharmaceutically acceptable salts thereof.

Other particular examples of compounds of formula I as described herein are selected from
2-[5-[2-hydroxy-6-methyl-4-(trifluoromethyl)phenyl]oxazolo[4,5-b]pyrazin-2-yl]-1,3-dihydropyrrolo[3,4-c]pyridin-6-ol; 2,2,2-trifluoroacetic acid;
2-[5-[2-hydroxy-6-methyl-4-(trifluoromethyl)phenyl]oxazolo[4,5-b]pyrazin-2-yl]-1,3-dihydropyrrolo[3,4-c]pyridin-6-ol;
6-[5-[2-hydroxy-6-methyl-4-(trifluoromethyl)phenyl]oxazolo[4,5-b]pyridin-2-yl]-5,7-dihydro-1H-pyrrolo[3,4-d]pyrimidine-2,4-dione;
   and pharmaceutically acceptable salts thereof.

Preferred examples of compounds of formula I as described herein are selected from
2-[5-[2-Hydroxy-6-methyl-4-(trifluoromethyl)phenyl]oxazolo[4,5-b]pyridin-2-yl]-3,5-dihydro-1H-pyrrolo[3,4-c]pyridin-6-one;
2-[5-[2-Hydroxy-6-methyl-4-(trifluoromethyl)phenyl]oxazolo[4,5-b]pyridin-2-yl]-5-methyl-1,3-dihydropyrrolo[3,4-c]pyridin-6-one;
3-Hydroxy-5-methyl-4-[2-(6-oxo-3,5-dihydro-1H-pyrrolo[3,4-c]pyridin-2-yl)oxazolo[4,5-b]pyridin-5-yl]benzonitrile;
2-[5-(4-Chloro-2-hydroxy-6-methyl-phenyl)oxazolo[4,5-b]pyridin-2-yl]-3,5-dihydro-1H-pyrrolo[3,4-c]pyridin-6-one;
5-(2-Hydroxyethyl)-2-[5-[2-hydroxy-6-methyl-4-(trifluoromethyl)phenyl]oxazolo[4,5-b]pyridin-2-yl]-1,3-dihydropyrrolo[3,4-c]pyridin-6-one;
5-chloro-2-[2-(1,3-dihydropyrrolo[3,4-c]pyridin-2-yl)oxazolo[4,5-b]pyridin-5-yl]-3-methyl-phenol;
5-chloro-3-methyl-2-[2-(2-methyl-7,8-dihydro-5H-pyrido[4,3-d]pyrimidin-6-yl)oxazolo[4,5-b]pyridin-5-yl]phenol;
5-chloro-2-[2-(5,7-dihydropyrrolo[3,4-d]pyrimidin-6-yl)oxazolo[4,5-b]pyridin-5-yl]-3-methyl-phenol; formic acid;
5-chloro-2-[2-(5,7-dihydropyrrolo[3,4-d]pyrimidin-6-yl)oxazolo[4,5-b]pyridin-5-yl]-3-methyl-phenol;
5-chloro-2-[2-(4,6-dihydro-1H-pyrrolo[3,4-c]pyrazol-5-yl)oxazolo[4,5-b]pyridin-5-yl]-3-methyl-phenol;
5-chloro-2-[2-(3,4-dihydro-1H-2,7-naphthyridin-2-yl)oxazolo[4,5-b]pyridin-5-yl]-3-methyl-phenol;
2-[2-(7,8-dihydro-5H-pyrido[4,3-d]pyrimidin-6-yl)oxazolo[4,5-b]pyridin-5-yl]-3-methyl-5-(trifluoromethyl)phenol;
5-chloro-3-methyl-2-[2-(2,4,6,7-tetrahydropyrazolo[4,3-c]pyridin-5-yl)oxazolo[4,5-b]pyridin-5-yl]phenol;
5-Chloro-2-[2-(1,3-dihydropyrrolo[3,4-c]pyridin-2-yl)oxazolo[4,5-b]pyrazin-5-yl]-3-methyl-phenol; formic acid;
5-Chloro-2-[2-(1,3-dihydropyrrolo[3,4-c]pyridin-2-yl)oxazolo[4,5-b]pyrazin-5-yl]-3-methyl-phenol;
6-[5-(4-Chloro-2-hydroxy-6-methyl-phenyl)oxazolo[4,5-b]pyridin-2-yl]-1,5,7,8-tetrahydropyrido[4,3-d]pyrimidin-2-one;
6-[5-(4-Chloro-2-hydroxy-6-methyl-phenyl)oxazolo[4,5-b]pyridin-2-yl]-5,7-dihydro-2H-pyrrolo[3,4-c]pyridazin-3-one;
   and pharmaceutically acceptable salts thereof.

More preferred examples of compounds of formula 1 as described herein are selected from
2-[5-[2-Hydroxy-6-methyl-4-(trifluoromethyl)phenyl]oxazolo[4,5-b]pyridin-2-yl]-3,5-dihydro-1H-pyrrolo[3,4-c]pyridin-6-one;
3-Hydroxy-5-methyl-4-[2-(6-oxo-3,5-dihydro-1H-pyrrolo[3,4-c]pyridin-2-yl)oxazolo[4,5-b]pyridin-5-yl]benzonitrile;
2-[5-(4-Chloro-2-hydroxy-6-methyl-phenyl)oxazolo[4,5-b]pyridin-2-yl]-3,5-dihydro-1H-pyrrolo[3,4-c]pyridin-6-one;
5-(2-Hydroxyethyl)-2-[5-[2-hydroxy-6-methyl-4-(trifluoromethyl)phenyl]oxazolo[4,5-b]pyridin-2-yl]-1,3-dihydropyrrolo[3,4-c]pyridin-6-one;
5-chloro-3-methyl-2-[2-(2-methyl-7,8-dihydro-5H-pyrido[4,3-d]pyrimidin-6-yl)oxazolo[4,5-b]pyridin-5-yl]phenol;
5-chloro-2-[2-(3,4-dihydro-1H-2,7-naphthyridin-2-yl)oxazolo[4,5-b]pyridin-5-yl]-3-methyl-phenol;
2-[2-(7,8-dihydro-5H-pyrido[4,3-d]pyrimidin-6-yl)oxazolo[4,5-b]pyridin-5-yl]-3-methyl-5-(trifluoromethyl)phenol;
5-chloro-3-methyl-2-[2-(2,4,6,7-tetrahydropyrazolo[4,3-c]pyridin-5-yl)oxazolo[4,5-b]pyridin-5-yl]phenol;
5-Chloro-2-[2-(1,3-dihydropyrrolo[3,4-c]pyridin-2-yl)oxazolo[4,5-b]pyrazin-5-yl]-3-methyl-phenol; formic acid;
5-Chloro-2-[2-(1,3-dihydropyrrolo[3,4-c]pyridin-2-yl)oxazolo[4,5-b]pyrazin-5-yl]-3-methyl-phenol;
6-[5-(4-Chloro-2-hydroxy-6-methyl-phenyl)oxazolo[4,5-b]pyridin-2-yl]-1,5,7,8-tetrahydropyrido[4,3-d]pyrimidin-2-one;
6-[5-(4-Chloro-2-hydroxy-6-methyl-phenyl)oxazolo[4,5-b]pyridin-2-yl]-5,7-dihydro-2H-pyrrolo[3,4-c]pyridazin-3-one;
   and pharmaceutically acceptable salts thereof.

Most preferred examples of compounds of formula 1 as described herein are selected from
2-[5-[2-Hydroxy-6-methyl-4-(trifluoromethyl)phenyl]oxazolo[4,5-b]pyridin-2-yl]-3,5-dihydro-1H-pyrrolo[3,4-c]pyridin-6-one;
3-Hydroxy-5-methyl-4-[2-(6-oxo-3,5-dihydro-1H-pyrrolo[3,4-c]pyridin-2-yl)oxazolo[4,5-b]pyridin-5-yl]benzonitrile;
2-[5-(4-Chloro-2-hydroxy-6-methyl-phenyl)oxazolo[4,5-b]pyridin-2-yl]-3,5-dihydro-1H-pyrrolo[3,4-c]pyridin-6-one;
5-(2-Hydroxyethyl)-2-[5-[2-hydroxy-6-methyl-4-(trifluoromethyl)phenyl]oxazolo[4,5-b]pyridin-2-yl]-1,3-dihydropyrrolo[3,4-c]pyridin-6-one;
5-Chloro-2-[2-(1,3-dihydropyrrolo[3,4-c]pyridin-2-yl)oxazolo[4,5-b]pyrazin-5-yl]-3-methyl-phenol; formic acid;
5-Chloro-2-[2-(1,3-dihydropyrrolo[3,4-c]pyridin-2-yl)oxazolo[4,5-b]pyrazin-5-yl]-3-methyl-phenol;
   and pharmaceutically acceptable salts thereof.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

An embodiment of the present invention is a compound according to formula I as described herein for use as a therapeutically active substance.

An embodiment of the present invention is a compound according to formula I as described herein for use in the treatment or prevention of a disease, disorder or condition, wherein the disease, disorder or condition is responsive to NLRP3 inhibition.

An embodiment of the present invention is a compound according to formula I as described herein for the treatment or prophylaxis of a disease, disorder or condition, wherein the disorder or condition is responsive to NLRP3 inhibition.

As used herein, the term “NLRP3 inhibition” refers to the complete or partial reduction in the level of activity of NLRP3 and includes, for example, the inhibition of active NLRP3 and/or the inhibition of activation of NLRP3.

There is evidence for a role of NLRP3-induced IL-1 and IL-18 in the inflammatory responses occurring in connection with, or as a result of, a multitude of different disorders (Menu et al., Clinical and Experimental Immunology, 166: 1-15, 2011; Strowig et al., Nature, 481: 278-286, 2012).

In one embodiment, the disease, disorder or condition is selected from:
(i) inflammation;
(ii) an auto-immune disease;
(iii) cancer;
(iv) an infection;
(v) a central nervous system disease;
(vi) a metabolic disease;
(vii) a cardiovascular disease;
(viii) a respiratory disease;
(ix) a liver disease;
(x) a renal disease;
(xi) an ocular disease;
(xii) a skin disease;
(xiii) a lymphatic condition;
(xiv) a psychological disorder;
(xv) graft versus host disease;
(xvi) allodynia;
(xvii) a condition associated with diabetes; and
(xviii) any disease where an individual has been determined to carry a germline or somatic non-silent mutation in NLRP3

In another embodiment, the disease, disorder or condition is selected from:
(i) cancer;
(ii) an infection;
(iii) a central nervous system disease;
(iv) a cardiovascular disease;
(v) a liver disease;
(vi) an ocular disease; and
(vii) a skin disease.

In a further typical embodiment of the invention, the disease, disorder or condition is inflammation. Examples of inflammation that may be treated or prevented include inflammatory responses occurring in connection with, or as a result of:
(i) a skin condition such as contact hypersensitivity, bullous pemphigoid, sunburn, psoriasis, atopical dermatitis, contact dermatitis, allergic contact dermatitis, seborrhoetic dermatitis, lichen planus, scleroderma, pemphigus, epidermolysis bullosa, urticaria, erythemas, or alopecia;
(ii) a joint condition such as osteoarthritis, systemic juvenile idiopathic arthritis, adult-onset Still's disease, relapsing polychondritis, rheumatoid arthritis, juvenile chronic arthritis, gout, or a seronegative spondyloarthropathy (e.g. ankylosing spondylitis, psoriatic arthritis or Reiter's disease);
(iii) a muscular condition such as polymyositis or myasthenia gravis;
(iv) a gastrointestinal tract condition such as inflammatory bowel disease (including Crohn's disease and ulcerative colitis), colitis, gastric ulcer, Coeliac disease, proctitis, pancreatitis, eosinopilic gastro-enteritis, mastocytosis, antiphospholipid syndrome, or a food-related allergy which may have effects remote from the gut (e.g., migraine, rhinitis or eczema);
(v) a respiratory system condition such as chronic obstructive pulmonary disease (COPD), asthma (including eosinophilic, bronchial, allergic, intrinsic, extrinsic or dust asthma, and particularly chronic or inveterate asthma, such as late asthma and airways hyper-responsiveness), bronchitis, rhinitis (including acute rhinitis, allergic rhinitis, atrophic rhinitis, chronic rhinitis, rhinitis caseosa, hypertrophic rhinitis, rhinitis pumlenta, rhinitis sicca, rhinitis medicamentosa, membranous rhinitis, seasonal rhinitis e.g. hay fever, and vasomotor rhinitis), sinusitis, idiopathic pulmonary fibrosis (IPF), sarcoidosis, farmer's lung, silicosis, asbestosis, volcanic ash induced inflammation, adult respiratory distress syndrome, hypersensitivity pneumonitis, or idiopathic interstitial pneumonia;
(vi) a vascular condition such as atherosclerosis, Behcet's disease, vasculitides, or Wegener's granulomatosis;
(vii) an autoimmune condition such as systemic lupus erythematosus, Sjögren's syndrome, systemic sclerosis, Hashimoto's thyroiditis, type I diabetes, idiopathic thrombocytopenia purpura, or Graves disease;
(viii) an ocular condition such as uveitis, allergic conjunctivitis, or vernal conjunctivitis;
(ix) a nervous condition such as multiple sclerosis or encephalomyelitis;
(x) an infection or infection-related condition, such as Acquired Immunodeficiency Syndrome (AIDS), acute or chronic bacterial infection, acute or chronic parasitic infection, acute or chronic viral infection, acute or chronic fungal infection, meningitis, hepatitis (A, B or C, or other viral hepatitis), peritonitis, pneumonia, epiglottitis, malaria, dengue hemorrhagic fever, leishmaniasis, streptococcal myositis, Mycobacterium tuberculosis (including Mycobacterium tuberculosis and HIV co-infection), Mycobacterium avium intracellulare, Pneumocystis carinii pneumonia, orchitis/epidydimitis, legionella, Lyme disease, influenza A, Epstein-Barr virus infection, viral encephalitis/aseptic meningitis, or pelvic inflammatory disease;
(xi) a renal condition such as mesangial proliferative glomerulonephritis, nephrotic syndrome, nephritis, glomerular nephritis, obesity related glomerulopathy, acute renal failure, acute kidney injury, uremia, nephritic syndrome, kidney fibrosis including chronic crystal nephropathy, or renal hypertension;
(xii) a lymphatic condition such as Castleman's disease;
(xiii) a condition of, or involving, the immune system, such as hyper IgE syndrome, lepromatous leprosy, familial hemophagocytic lymphohistiocytosis, or graft versus host disease;
(xiv) a hepatic condition such as chronic active hepatitis, non-alcoholic steatohepatitis (NASH), alcohol-induced hepatitis, non-alcoholic fatty liver disease (NAFLD), alcoholic fatty liver disease (AFLD), alcoholic steatohepatitis (ASH), primary biliary cirrhosis, fulminant hepatitis, liver fibrosis, or liver failure;
(xv) a cancer, including those cancers listed above;
(xvi) a burn, wound, trauma, haemorrhage or stroke;
(xvii) radiation exposure;
(xviii) a metabolic disease such as type 2 diabetes (T2D), atherosclerosis, obesity, gout or pseudo-gout; and/or
(xix) pain such as inflammatory hyperalgesia, pelvic pain, allodynia, neuropathic pain, or cancer-induced bone pain.

An embodiment of the present invention is a compound according to formula I as described herein for the treatment or prophylaxis of a disease, disorder or condition selected from:
inflammation;
an auto-immune disease;
cancer;
an infection;
a central nervous system disease;
a metabolic disease;
a cardiovascular disease;
a respiratory disease;
a liver disease;
a renal disease;
an ocular disease;
a skin disease;
a lymphatic condition;
a psychological disorder;
graft versus host disease;
allodynia;
a condition associated with diabetes; and
any disease where an individual has been determined to carry a germline or somatic non-silent mutation in NLRP3.

An embodiment of the present invention is the use of a compound according to formula I as described herein in the treatment or prophylaxis of a disease, disorder or condition, wherein the disease, disorder or condition is responsive to NLRP3 inhibition.

An embodiment of the present invention is the use of a compound according to formula I as described herein in the treatment or prophylaxis of a disease, disorder or condition selected from Alzheimer's disease and Parkinson's disease.

An embodiment of the present invention is the use of a compound according to formula I as described herein in the treatment or prophylaxis of a disease, disorder or condition selected from Multiple Sclerosis (MS) and Amyotrophic lateral sclerosis (ALS).

An embodiment of the present invention is the use a compound according to formula I as described herein for use in the treatment or prophylaxis of a disease, disorder or condition selected from Asthma and COPD.

An embodiment of the present invention is the use a compound according to formula I as described herein for use in the treatment or prophylaxis of a disease, disorder or condition selected from Cryopyrin-associated periodic syndromes.

An embodiment of the present invention is a compound according to formula I as described herein for the treatment or prophylaxis of a disease, disorder or condition selected from Alzheimer's disease and Parkinson's disease.

An embodiment of the present invention is a compound according to formula I as described herein for the treatment or prophylaxis of a disease, disorder or condition selected from Multiple Sclerosis (MS) and Amyotrophic lateral sclerosis (ALS).

An embodiment of the present invention is a compound according to formula I as described herein for the treatment or prophylaxis of a disease, disorder or condition selected from Asthma and COPD.

An embodiment of the present invention is a compound according to formula I as described herein for the treatment or prophylaxis of a disease, disorder or condition selected from Cryopyrin-associated periodic syndromes.

An embodiment of the present invention is the use of a compound according to formula I as described herein for preparation of a medicament for the treatment or prophylaxis of a disease, disorder or condition selected from Alzheimer's disease and Parkinson's disease.

An embodiment of the present invention is the use of a compound according to formula I as described herein for preparation of a medicament for the treatment or prophylaxis of a disease, disorder or condition selected from Multiple Sclerosis (MS) and Amyotrophic lateral sclerosis (ALS).

An embodiment of the present invention is the use of a compound according to formula I as described herein for the preparation of a medicament for the treatment or prophylaxis of a disease, disorder or condition selected from Asthma and COPD.

An embodiment of the present invention is the use of a compound according to formula I as described herein for the preparation of a medicament for the treatment or prophylaxis of a disease, disorder or condition selected from Cryopyrin-associated periodic syndromes.

An embodiment of the present invention is a method of treatment or prophylaxis of a disease, disorder or condition selected from Alzheimer's disease and Parkinson's disease, which method comprises administering an effective amount of a compound according to formula I as described herein.

An embodiment of the present invention is a method of treatment or prophylaxis of a disease, disorder or condition selected from Multiple Sclerosis (MS) and Amyotrophic lateral sclerosis (ALS), which method comprises administering an effective amount of a compound according to formula I as described herein.

An embodiment of the present invention is a method of treatment or prophylaxis of a disease, disorder or condition selected from Asthma and COPD, which method comprises administering an effective amount of a compound according to formula I as described herein.

An embodiment of the present invention is a method of treatment or prophylaxis of a disease, disorder or condition selected from Cryopyrin-associated periodic syndromes, which method comprises administering an effective amount of a compound according to formula I as described herein.

An embodiment of the present invention relates to a method of inhibiting NLRP3, which method comprises administering an effective amount of a compound according to formula I as described herein.

Also an embodiment of the present invention are compounds of formula I as described herein, when manufactured according to any one of the described processes.

An embodiment of the present invention is a pharmaceutical composition comprising a compound according to formula I as described herein and a therapeutically inert carrier.

### Assay Procedures

### NLRP3 and Pyroptosis

It is well established that the activation of NLRP3 leads to cell pyroptosis and this feature plays an important part in the manifestation of clinical disease (Yan-gang Liu et al., Cell Death & Disease, 2017, 8(2), e2579; Alexander Wree et al., Hepatology, 2014, 59(3), 898-910; Alex Baldwin et al., Journal of Medicinal Chemistry, 2016, 59(5), 1691-1710; Ema Ozaki et al., Journal of Inflammation Research, 2015, 8, 15-27; Zhen Xie & Gang Zhao, Neuroimmunology Neuroinflammation, 2014, 1(2), 60-65; Mattia Cocco et al., Journal of Medicinal Chemistry, 2014, 57(24), 10366-10382; T. Satoh et al., Cell Death & Disease, 2013, 4, e644). Therefore, it is anticipated that inhibitors of NLRP3 will block pyroptosis, as well as the release of pro-inflammatory cytokines (e.g. IL-1β) from the cell.

### THP-1 Cells: Culture and Preparation

THP-1 cells (ATCC #TIB-202) were grown in RPMI containing L-glutamine (Gibco #11835) supplemented with 1 mM sodium pyruvate (Sigma #S8636) and penicillin (100 units/ml)/streptomycin (0.1 mg/ml) (Sigma #P4333) in 10% Fetal Bovine Serum (FBS) (Sigma #F0804). The cells were routinely passaged and grown to confluency (˜10⁶ cells/ml). On the day of the experiment, THP-1 cells were harvested and resuspended into RPMI medium (without FBS). The cells were then counted and viability (>90%) checked by Trypan blue (Sigma #T8154). Appropriate dilutions were made to give a concentration of 625,000 cells/ml. To this diluted cell solution was added LPS (Sigma #L4524) to give a 1 μg/ml Final Assay Concentration (FAC). 40 μl of the final preparation was aliquoted into each well of a 96-well plate. The plate thus prepared was used for compound screening.

### THP-1 Cells Pyroptosis Assay

The following method step-by-step assay was followed for compound screening.
Seed THP-1 cells (25,000 cells/well) containing 1.0 μg/ml LPS in 40 μl of RPMI medium (without FBS) in 96-well, black walled, clear bottom cell culture plates coated with poly-D-lysine (VWR #734-0317)
Add 5 μl compound (8 points half-log dilution, with 10 μM top dose) or vehicle (DMSO 0.1% FAC) to the appropriate wells
Incubate for 3 hours at 37° C., 5% CO₂
Add 5 μl nigericin (Sigma #N7143) (FAC 5 μM) to all wells
Incubate for 1 hr at 37° C., 5% CO₂
At the end of the incubation period, spin plates at 300×g for 3 mins and remove supernatant
Then add 50 μl of resazurin (Sigma #R7017) (FAC 100 μM resazurin in RPMI medium without FBS) and incubate plates for a further 1-2 hours at 37° C. and 5% CO₂
Plates were read in an Envision reader at Ex 560 nm and Em 590 nm
IC₅₀ data is fitted to a non-linear regression equation (log inhibitor vs response-variable slope 4-parameters)

The results of the pyroptosis assay are summarised in Table 1 below as THP IC₅₀.

### Human Whole Blood IL-1β Release Assay

For systemic delivery, the ability to inhibit NLRP3 when the compounds are present within the bloodstream is of great importance. For this reason, the NLRP3 inhibitory activity of a number of compounds in human whole blood was investigated in accordance with the following protocol.
Human whole blood in Li-heparin tubes was obtained from healthy donors from a volunteer donor panel.
Plate out 80 μl of whole blood containing 1 μg/ml of LPS in 96-well, clear bottom cell culture plate (Corning #3585)
Add 10 μl compound (8 points half-log dilution with 10 μM top dose) or vehicle (DMSO 0.1% FAC) to the appropriate wells
Incubate for 3 hours at 37° C., 5% CO₂
Add 10 μl nigericin (Sigma #N7143) (10 μM FAC) to all wells
Incubate for 1 hr at 37° C., 5% CO₂
At the end of the incubation period, spin plates at 300×g for 5 mins to pellet cells and remove 20 μl of supernatant and add to 96-well v-bottom plates for IL-1β analysis (note: these plates containing the supernatants can be stored at −80° C. to be analysed at a later date)
IL-1β was measured according to the manufacturer protocol (Perkin Elmer-AlphaLisa IL-1 Kit AL220F-5000)
IC₅₀ data is fitted to a non-linear regression equation (log inhibitor vs response-variable slope 4-parameters)

The results of the human whole blood assay are summarised in Table 1 below as HWB IC₅₀.

### Microsomal Stability:

Incubations of test compounds at 1 μM in microsomes (0.5 mg/mL) plus cofactor NADPH are performed in 96 well plates at 37° C. on a TECAN (Tecan Group Ltd, Switzerland) automated liquid handling system. After a 10 minutes pre-incubation step of the test compound with the microsomes, the enzymatic reaction is started by the addition of cofactors. At 1, 3, 6, 9, 15, 25, 35 and 45 minutes, aliquots of the incubations are removed and quenched with 1:3 (v/v) acetonitrile containing internal standard. Samples are then cooled and centrifuged before analysis of the supernatant by LC-MS/MS 2.

### Metabolic Stability in Hepatocytes:

### Assay Descriptions:

Biological materials. Cryopreserved hepatocytes [mouse, rat, rabbit, monkey and human (male and female; mixed)] are obtained. Viability of hepatocytes after reconstitution is at least 80% throughout the study. Ready-to-use rat/human HepatoPac® cultures [long-term hepatocyte co-cultures; pooled (n=5 for male and n=5 for female for human)] with stromal mouse fibroblasts (negative control; pooled) with the plates for incubations, application medium and maintenance medium are acquired.

Metabolism by suspended hepatocytes. Primary pooled cryopreserved hepatocytes are reconstituted in pre-warmed William's E media containing 10% FCS, 0.05 mg/mL streptomycin and 50 U/mL penicillin and 0.4 mM L-glutamine; and 0.01 mg/mL gentamicin, 0.048 mg/mL hydrocortisone and 0.004 mg/mL insulin, to a final suspension density of 1×106 cells/mL. The incubation was performed fully automatically with Liquid Handling System (Tecan) equipped with a CO2 incubator with an orbital shaker. After the addition of a test compound at e.g. 1 μM to the wells (1×105 cells/well), the 96-well hepatocyte suspension culture plates are incubated in a 5% CO2 at 37° C. Samples are quenched by addition of acetonitrile (including an internal standard) to the incubation well at the designated time points up to 2 h.

Metabolism by HepatoPac®. Incubations for a test article (at e.g. 1 μM, 0.1% v/v DMSO) as conducted in suspension assays are performed in 96-well plates containing either a co-culture of adherent hepatocytes with mouse fibroblast control cells or control cells alone (5% CO2 atmosphere and 37° C.). The incubation media in human HepatoPac® is identical with that in suspended hepatocytes. At defined time points (2, 18, 26, 48, 72 and 96 h), whole wells are quenched with ice-cold acetonitrile containing an internal standard.

Samples are then centrifuged appropriately and the supernatant analyzed by LC-MS/MS. The incubation is conducted in n=1 or 2.

TABLE 1
NLRP3 inhibitory activity

| Example | THP-1 pyroptosis | Human whole blood |
|---|---|---|
| No. | Assay IC₅₀ (nM) | IL-1β Assay IC₅₀ (nM) |
| 1 | 0.7 | 108.5 |
| 2 | 3.3 | 300.7 |
| 3 | 7.7 | 116.9 |
| 4 | 1.7 | 77.0 |
| 5 | 2.6 | 151.2 |
| 6 | 90.0 | |
| 7 | 9.5 | 486.8 |
| 8 | 69.9 | |
| 9 | 262.6 | |
| 10 | 29.0 | 2231.0 |
| 11 | 27.1 | 225.4 |
| 12 | 2.0 | 839.6 |
| 13 | 15.0 | 303.6 |
| 14 | 56.2 | 305.1 |
| 15 | 10.5 | 173.5 |
| 16 | 66.0 | |
| 17 | 34.9 | 225.1 |
| 18 | 6.9 | 2345.6 |
| 19 | 11.1 | 962.1 |
| 20 | 4.2 | 1446.7 |
| 21 | 22.3 | 1957.9 |
| 22 | 20.7 | 10000.0 |
| 23 | | 3717.2 |
| 24 | 23.6 | 191.9 |
| 25 | 25.6 | 141.7 |
| 26 | 46.7 | 166.3 |
| 27 | 52.2 | 423.1 |
| 29 | 2.1 | 35.9 |
| 30 | 24.8 | 326.1 |
| 31 | 17.9 | 344.9 |
| 32 | 24.9 | 284.9 |
| 33 | 447.2 | |
| 34 | 675.3 | |
| 35 | 236.0 | |
| 36 | 46.3 | 168.5 |
| 37 | 71.1 | 676.3 |
| 38 | 22.0 | 168.3 |
| 40 | 71.6 | |
| 41 | 174.4 | |
| 42 | 144.5 | |
| 43 | 1.7 | 128.3 |
| 44 | 245 | |

The invention will now be illustrated by the following examples which have no limiting character.

In case the preparative examples are obtained as a mixture of enantiomers or diastereoisomers, the pure enantiomers or diastereomers can be obtained by methods described herein or by methods known to those skilled in the art, such as e.g. chiral chromatography or crystallization.

### EXPERIMENTAL METHODS

Abbreviations

| | |
|---|---|
| AcOK | Potassium acetate |
| Aq. | Aqueous |
| BnBr | Benzyl bromide |
| t-BuONO | tert-butyl nitrite |
| DAST | Diethylaminosulfur trifluoride |
| DCM | Dichloromethane |
| DIPEA | N,N-Diisopropylethylamine |
| DMF | Dimethylformamide |
| ESI | Electrospray ionization |
| EtOH | Ethanol |
| EtOAC | Ethyl acetate |
| eq | Equivalent |
| FA | Formic acid |
| H, hrs | Hour(s) |
| HPLC | High-performance liquid chromatography |
| LCMS | Liquid chromatography-mass spectrometry |
| MeCN | Acetonitrile |
| MeOH | Methanol |
| NCS | N-Chlorosuccinimid |
| PE | Petroleum ether |
| TBSCl | tert-butyldimethylsilyl chloride |
| TFA | Trifluoroacetic acid |
| THF | Tetrahydrofuran |
| TLC | Thin layer chromatography |
| SEMCl | 2-(Trimethylsilyl)ethoxymethyl chloride |

### EXAMPLES

All examples and intermediates were prepared under nitrogen atmosphere if not specified otherwise.

### Preparation of Intermediates

### Intermediate 1

### 5-(4-Chloro-2-hydroxy-6-methyl-phenyl)-3H-oxazolo[4,5-b]pyridine-2-thione

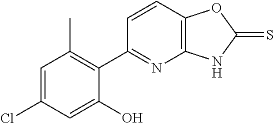 [SMILES: Cc1cc(Cl)cc(O)c1-c1ccc2oc(=S)[nH]c2n1]

### Step A: 2-Amino-6-(4-chloro-2-hydroxy-6-methyl-phenyl)pyridin-3-ol

(4-Chloro-2-hydroxy-6-methyl-phenyl)boronic acid (CAS #1207961-50-9, 1972.4 mg, 10.6 mmol, 1.0 eq), XPhos Pd G3 (269.02 mg, 0.32 mmol, 0.03 eq), sat. aq. NaHCO₃ (5 mL) and 2-amino-6-bromopyridin-3-ol (CAS #934758-27-7, 2000.0 mg, 10.6 mmol, 1.0 eq) were suspended in dioxane (40 mL) and degassed with N₂ (5 min). The reaction mixture was heated to 80° C. and stirred for 18 h. The reaction mixture was filtered through a plug of celite, then dry-loaded onto silica and purified by flash chromatography on silica gel (80 g column, 0-10% MeOH/DCM) to afford the title compound (1001 mg, 37% yield) as a light brown solid. LCMS m/z 251.1 [M+H]⁺ ESI pos.

### Step B: 5-(4-Chloro-2-hydroxy-6-methyl-phenyl)-3H-oxazolo[4,5-b]pyridine-2-thione

1,1′-Thiocarbonyldiimidazole (938.4 mg, 5.27 mmol, 1.2 eq) was added to a stirred solution of 2-amino-6-(4-chloro-2-hydroxy-6-methyl-phenyl)pyridin-3-ol (1100.0 mg, 4.39 mmol, 1.0 eq) in DMF (20 mL) at room temperature and the reaction mixture was stirred for 18 h. The mixture was poured into 1 N aq. HCl (100 mL). The mixture was extracted EtOAc (2×100 mL). The combined organic layers were washed with 10 wt % aqueous LiCl (3×50 mL), dried (phase separator) and concentrated in vacuo to afford the title compound (1102.0 mg, 77% yield) as a light brown solid. LCMS m/z 293.0 [M+H]⁺ ESI pos.

### Intermediate 2

### 5-[2-Hydroxy-6-methyl-4-(trifluoromethyl)phenyl]-3H-oxazolo[4,5-b]pyridine-2-thione

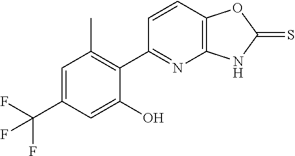 [SMILES: Cc1cc(C(F)(F)F)cc(O)c1-c1ccc2oc(=S)[nH]c2n1]

### Step A: 2-Amino-6-[2-hydroxy-6-methyl-4-(trifluoromethyl)phenyl]pyridin-3-ol

A solution of 2-amino-6-bromopyridin-3-ol (250.0 mg, 1.32 mmol, 1.0 eq), 3-methyl-2-(4,4,5,5-tetramethyl-1,3,2-dioxaborolan-2-yl)-5-(trifluoromethyl)phenol (359.6 mg, 1.19 mmol, 0.9 eq) and XPhos Pd G3 (112.09 mg, 0.13 mmol, 0.1 eq) in 1,4-dioxane (1 mL) was sparged with nitrogen for 5 mins. sat. aq. Na₂CO₃ (0.5 mL) and water (0.1 mL) were added after which the reaction mixture was heated to 80° C. and stirred at this temperature for 18 hours. The reaction mixture was cooled to r.t. after which it was concentrated in vacuo. The resulting residue was dissolved in EtOAc (20 mL) and filtered through a pad of celite. The filtrate was diluted with 1:1 water:brine (20 mL) after which the layers were separated. The aqueous layer was extracted with EtOAc (2×20 mL) after which the combined organics were concentrated in vacuo. The crude product was dry loaded onto celite and purified by silica gel chromatography (12 g cartridge, 0-100% ((3:1 EtOAc:EtOH with 2% NH₃OH)/isohexane)) to afford the title compound (119.0 mg, 31% yield) as a light grey solid. LCMS m/z 285.1 [M+H]⁺ ESI pos.

### Step B: 5-[2-Hydroxy-6-methyl-4-(trifluoromethyl)phenyl]-3H-oxazolo[4,5-b]pyridine-2-thione

A solution of 1,1′-thiocarbonyldiimidazole (89.5 mg, 0.5 mmol, 1.2 eq) and 2-amino-6-[2-hydroxy-6-methyl-4-(trifluoromethyl)phenyl]pyridin-3-ol (119.0 mg, 0.42 mmol, 1.0 eq) in DMF (3 mL) was stirred at r.t. for 18 h. The reaction mixture was diluted with EtOAc (10 mL) and 1:1 water:brine (10 mL) after which the layers were separated. The aqueous layer was extracted with EtOAc (2×10 mL) after which the combined organics were concentrated in vacuo. The crude product was triturated with isohexane (10 mL) to afford the title compound (156 mg, 78% yield) as a pale brown solid. LCMS m/z 327.1 [M+H]⁺ ESI pos.

### PREPARATION OF EXAMPLES

### Example 1

### 2-[5-[2-Hydroxy-6-methyl-4-(trifluoromethyl)phenyl]oxazolo[4,5-b]pyridin-2-yl]-3,5-dihydro-1H-pyrrolo[3,4-c]pyridin-6-one

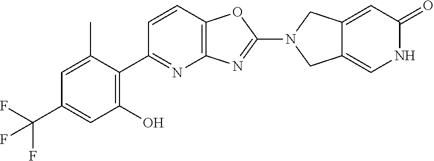 [SMILES: Cc1cc(C(F)(F)F)cc(O)c1-c1ccc2oc(N3Cc4c[nH]c(=O)cc4C3)nc2n1]

### Step A: tert-Butyl 6-benzyloxy-1,3-dihydropyrrolo[3,4-c]pyridine-2-carboxylate

To a mixture of tert-butyl 6-chloro-1,3-dihydropyrrolo[3,4-c]pyridine-2-carboxylate (CAS #1700330-18-2, 988 mg, 3.68 mmol, 1.00 eq) and benzyl alcohol (603 mg, 0.58 mL, 5.58 mmol, 1.51 eq) in 1,4-dioxane (11 mL) was added potassium tert-butoxide (579 mg, 5.16 mmol, 1.40 eq). The reaction mixture was stirred at 100° C. for 16 hours. The reaction mixture was cooled to room temperature, quenched with water and extracted with ethyl acetate. The aqueous layer was backextracted with ethyl acetate. The organic layers were washed with brine. The combined organic layers were dried over sodium sulfate, filtered and concentrated in vacuo. The residue was adsorbed on ISOLUTE HM-N and purified by flash chromatography (silica gel, 40 g, gradient 0% to 20% ethyl acetate in heptane) to afford the title compound (898 mg, 71% yield) as a purple solid. LCMS: m/z 327.2 [M+H]⁺, ESI pos.

### Step B: tert-Butyl 6-oxo-3,5-dihydro-1H-pyrrolo[3,4-c]pyridine-2-carboxylate

A solution of tert-butyl 6-benzyloxy-1,3-dihydropyrrolo[3,4-c]pyridine-2-carboxylate (Example 1, step A) (895 mg, 2.61 mmol, 1.00 eq) in ethyl acetate (10 mL) and methanol (10 mL) was three times alternating evacuated and flushed with argon. Palladium on activated charcoal, 10% Pd basis (90 mg, 0.08 mmol, 0.03 eq) was added carefully. The reaction flask was three times alternating evacuated and flushed with argon. Then, the reaction flask was evacuated again and flushed with hydrogen. The reaction mixture was stirred under hydrogen atmosphere (balloon) at room temperature for 4 hours. The reaction mixture was filtered and rinsed well with warm ethyl acetate/methanol. The filtrate was concentrated in vacuo to afford the title compound (654 mg, 96% yield, 90% purity) as an off-white solid, which was used without further purification. LCMS: m/z 237.1 [M+H]⁺, ESI pos.

### Step C: 2-(5-Chlorooxazolo[4,5-b]pyridin-2-yl)-3,5-dihydro-1H-pyrrolo[3,4-c]pyridin-6-one

To a solution of tert-butyl 6-oxo-3,5-dihydro-1H-pyrrolo[3,4-c]pyridine-2-carboxylate (Example 1, step B) (60 mg, 0.24 mmol, 1.13 eq) in dichloromethane (0.70 mL) and methanol (0.30 mL) was added dropwise 4 M HCl in dioxane (720 mg, 0.60 mL, 2.40 mmol, 11.3 eq). Let stir at room temperature for 1.5 hours. The reaction mixture was concentrated in vacuo. The residue was taken up in 1,4-dioxane (0.50 mL) and 5-chloro-2-(methylthio)oxazolo[4,5-b]pyridine (CAS #1783370-92-2, 45 mg, 0.21 mmol, 1.00 eq) was added followed by triethylamine (73 mg, 0.10 mL, 0.72 mmol, 3.37 eq). The reaction mixture was stirred at 90° C. for 25 hours and then left standing at room temperature for 4 days. The reaction mixture was extracted three times with a mixture of dichloromethane/methanol (9:1) and half-saturated aq. NaHCO₃-solution (solids were present in the aqueous layer). The aqueous layer was filtered, rinsing with water. The filter cake was dried using the rotavap and then put under high vacuum to afford the title compound (20 mg, 31% yield) as a dark brown solid. LCMS: m/z 289.1 [M+H]⁺, ESI pos.

### Step D: 2-[5-[2-Hydroxy-6-methyl-4-(trifluoromethyl)phenyl]oxazolo[4,5-b]pyridin-2-yl]-3,5-dihydro-1H-pyrrolo[3,4-c]pyridin-6-one

A mixture of 2-(5-chlorooxazolo[4,5-b]pyridin-2-yl)-3,5-dihydro-1H-pyrrolo[3,4-c]pyridin-6-one (Example 1, step C) (20 mg, 0.07 mmol, 1.00 eq), 3-methyl-2-(4,4,5,5-tetramethyl-1,3,2-dioxaborolan-2-yl)-5-(trifluoromethyl)phenol (CAS #2557358-38-8, 38 mg, 0.11 mmol, 1.72 eq, 90% purity), cesium carbonate (65 mg, 0.20 mmol, 3.03 eq) and XPhos Pd G3 (9 mg, 0.01 mmol, 0.16 eq) in 1,4-dioxane (0.60 mL) and water (0.15 mL) was flushed with argon and stirred at 100° C. for 2 hours. The reaction mixture was cooled to room temperature and extracted with a mixture of dichloromethane/methanol (9:1) and half-saturated aq. NH₄Cl-solution. The aqueous layers were backextracted twice with a mixture of dichloromethane/methanol (9:1). The combined organic layers were dried over sodium sulfate, filtered and concentrated in vacuo. The crude product was adsorbed on ISOLUTE HM-N and purified by flash chromatography (silica gel, 12 g, gradient 0% to 10% methanol in dichloromethane) to afford the title compound (14 mg, 47% yield) as an off-white solid. LCMS: m/z 429.2 [M+H]⁺, ESI pos.

### Example 2

### 2-[5-[2-Hydroxy-6-methyl-4-(trifluoromethyl)phenyl]oxazolo[4,5-b]pyridin-2-yl]-5-methyl-1,3-dihydropyrrolo[3,4-c]pyridin-6-one

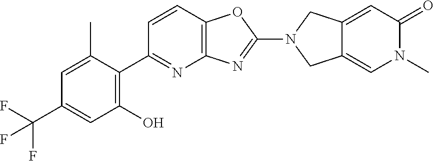 [SMILES: Cc1cc(C(F)(F)F)cc(O)c1-c1ccc2oc(N3Cc4cc(=O)n(C)cc4C3)nc2n1]

### Step A: tert-Butyl 5-methyl-6-oxo-1,3-dihydropyrrolo[3,4-c]pyridine-2-carboxylate

To a mixture of tert-butyl 6-oxo-3,5-dihydro-1H-pyrrolo[3,4-c]pyridine-2-carboxylate (Example 1, step B) (170 mg, 0.68 mmol, 1.00 eq) in N,N-dimethylformamide (0.80 mL) was added potassium carbonate (222 mg, 1.61 mmol, 2.35 eq) followed by iodomethane (227 mg, 0.10 mL, 1.60 mmol, 2.34 eq). The reaction mixture was stirred at room temperature for 16 hours. The reaction mixture was added onto saturated aq. NH₄Cl-solution and extracted with ethyl acetate. The aqueous layer was back extracted with ethyl acetate. The organic layers were washed with saturated aq. NH₄Cl-solution and water. The combined organic layers were dried over sodium sulfate, filtered and concentrated in vacuo. The crude product was adsorbed on ISOLUTE HM-N and purified by flash chromatography (silica gel, 12 g. gradient 0% to 10% methanol in dichloromethane) to afford the title compound (144 mg, 80% yield) as an off-white solid. LCMS: m/z 251.1 [M+H]⁺, ESI pos.

### Step B: 2-(5-Chlorooxazolo[4,5-b]pyridin-2-yl)-5-methyl-1,3-dihydropyrrolo[3,4-c]pyridin-6-one

To a solution of tert-butyl 5-methyl-6-oxo-1,3-dihydropyrrolo[3,4-c]pyridine-2-carboxylate (Example 2, step A) (106 mg, 0.40 mmol, 1.21 eq) in dichloromethane (1.0 mL) and methanol (0.50 mL) was added dropwise 4 M HCl in dioxane (1.20 g, 1.0 mL, 4.00 mmol, 12.1 eq). Let stir at room temperature for 4 hours. The reaction mixture was concentrated in vacuo. The residue was taken up in 1,4-dioxane (0.70 mL) and 5-chloro-2-(methylthio)oxazolo[4,5-b]pyridine (CAS #1783370-92-2, 70 mg, 0.33 mmol, 1.00 eq) was added followed by triethylamine (112 mg, 0.154 mL, 1.10 mmol, 3.33 eq). The reaction mixture was stirred at 90° C. for 2 days. The reaction mixture was cooled to room temperature and extracted three times with a mixture of dichloromethane/methanol (9:1) and half-saturated aq. NaHCO₃-solution. The combined organic layers were dried over sodium sulfate, filtered and concentrated in vacuo. The crude product was adsorbed on ISOLUTE HM-N and purified by flash chromatography (silica gel, 12 g. gradient 0% to 100% (dichloromethane:methanol:NH₄OH 9:1:0.05) in dichloromethane) to afford the title compound (20 mg, 19% yield) as an off-white solid. LCMS: m/z 303.1 [M+H]⁺, ESI pos.

### Step C: 2-[5-[2-Hydroxy-6-methyl-4-(trifluoromethyl)phenyl]oxazolo[4,5-b]pyridin-2-yl]-5-methyl-1,3-dihydropyrrolo[3,4-c]pyridin-6-one

A mixture of 2-(5-chlorooxazolo[4,5-b]pyridin-2-yl)-5-methyl-1,3-dihydropyrrolo[3,4-c]pyridin-6-one (Example 2, step B) (27 mg, 0.08 mmol, 1.00 eq), 3-methyl-2-(4,4,5,5-tetramethyl-1,3,2-dioxaborolan-2-yl)-5-(trifluoromethyl)phenol (CAS #2557358-38-8, 49 mg, 0.15 mmol, 1.72 eq, 90% purity), cesium carbonate (84 mg, 0.26 mmol, 3.04 eq) and XPhos Pd G3 (12 mg, 0.01 mmol, 0.17 eq) in 1,4-dioxane (0.80 mL) and water (0.20 mL) was flushed with argon and stirred at 100° C. for 3.5 h. The reaction mixture was cooled to room temperature and extracted with a mixture of dichloromethane/methanol (9:1) and half-saturated aq. NH₄Cl-solution. The aqueous layer was back extracted twice with a mixture of dichloromethane/methanol (9:1). The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in dichloromethane) to afford the title compound (25 mg, 63% yield) as an off-white solid. LCMS: m/z 443.2 [M+H]⁺, ESI pos.

### Example 3

### 3-Hydroxy-5-methyl-4-[2-(6-oxo-3,5-dihydro-1H-pyrrolo[3,4-c]pyridin-2-yl)oxazolo[4,5-b]pyridin-5-yl]benzonitrile

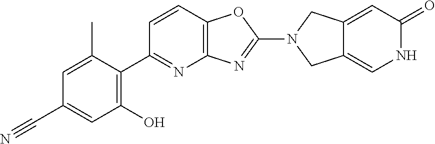 [SMILES: Cc1cc(C#N)cc(O)c1-c1ccc2oc(N3Cc4c[nH]c(=O)cc4C3)nc2n1]

### Step A: 2-(5-Bromooxazolo[4,5-b]pyridin-2-yl)-3,5-dihydro-1H-pyrrolo[3,4-c]pyridin-6-one

To a solution of tert-butyl 6-oxo-3,5-dihydro-1H-pyrrolo[3,4-c]pyridine-2-carboxylate (Example 1, step B) (165 mg, 0.63 mmol, 1.16 eq, 90% purity) in dichloromethane (1.6 mL) and methanol (0.80 mL) was added dropwise 4 M HCl in dioxane (1.80 g, 1.5 mL, 6.00 mmol, 11.1 eq). Let stir at room temperature for 3.5 hours. The reaction mixture was concentrated in vacuo. The residue was taken up in 1,4-dioxane (1.3 mL) and 5-bromo-2-(methylthio)oxazolo[4,5-b]pyridine (CAS #1780768-09-3, 140 mg, 0.54 mmol, 1.00 eq) was added followed by triethylamine (189 mg, 0.26 mL, 1.87 mmol, 3.44 eq). The reaction mixture was stirred at 95° C. for 2 days. The reaction mixture was diluted with a small amount of dichloromethane/methanol (19:1) and half-saturated aq. NaHCO₃-solution. The mixture was sonicated, filtered, rinsing with a minimal amount of water and a mixture of dichloromethane/methanol (19:1). The filter cake was dried using the rotavap and then put under high vacuum to afford the title compound (74 mg, 37% yield, 90% purity) as a grey solid. LCMS: m/z 333.0/335.0 [M+H]⁺, ESI pos.

### Step A′: 4-Amino-3-methoxy-5-methyl-benzonitrile

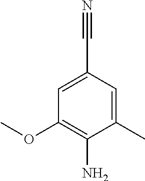

Two Batches were Carried Out in Parallel.

To a solution of commercially available 4-bromo-2-methoxy-6-methylbenzenamine (CAS #348169-39-1, 25.0 g, 115 mmol, 1.00 eq) in DMF (250 mL) was added Zn(CN)₂ (13.5 g, 115 mmol, 7.34 mL, 1.0 eq) and Pd(PPh₃)₄ (66.8 g, 57.8 mmol, 0.5 eq). The reaction mixture was stirred at 100° C. for 12 hrs. The reaction mixture was poured into water (1.50 L) and extracted with ethyl acetate (1 L×3). The organic phase was washed with brine (1 L×3), dried over Na₂SO₄, filtered and concentrated under reduced pressure to give a residue, which was purified by column chromatography (SiO₂, petroleum ether/ethyl acetate=100/1 to 0/1) to give the title compound (28.0 g, 75% yield) as a yellow solid. ¹H NMR (DMSO-d₆) δ 7.05 (s, 2H), 5.47 (bs, 2H), 3.81 (s, 3H), 2.09 (s, 3H).

### Step B′: 4-Bromo-3-methoxy-5-methyl-benzonitrile

To a solution of CuBr (46.4 g, 323 mmol, 9.86 mL, 1.50 eq) in MeCN (180 mL) was added t-BuONO (33.3 g, 323 mmol, 38.5 mL, 1.50 eq) and stirred at 65° C. Then solution of aforementioned Intermediate 2B 4-bromo-3-methoxy-5-methyl-benzonitrile (35.0 g, 215 mmol, 1.00 eq) in MeCN (180 mL) was added at 65° C. The mixture was stirred at 65° C. for 3.5 hrs. After completion, sat. aq. Na₂SO₃ (400 mL) and sat. aq. NH₄Cl (200 mL) was added to the mixture and extracted with ethyl acetate (500 mL×3). The organic phase was washed with brine (500 mL×2), dried over Na₂SO₄, filtered and concentrated under reduced pressure to give a residue. The residue was purified by column chromatography (SiO₂, petroleum ether/ethyl acetate=100/1 to 0/1, R_{f}=0.75) to give the title compound (20.7 g, 42% yield) as a white solid. ¹H NMR (DMSO-d₆) δ 7.43, 7.40 (2s, 1H each), 3.90 (s, 3H), 2.37 (s, 3H).

### Step C′: 3-Methoxy-5-methyl-4-(4,4,5,5-tetramethyl-1,3,2-dioxaborolan-2-yl)benzonitrile

To a solution of aforementioned 4-bromo-3-methoxy-5-methyl-benzonitrile (18.0 g, 79.6 mmol, 1.00 eq) in DMF (180 mL) was added B₂Pin₂ (30.3 g, 119 mmol, 1.50 eq) and AcOK (35.1 g, 358 mmol, 4.50 eq). The mixture was stirred at 20° C. for 0.5 hr and Pd(dppf)Cl₂·CH₂Cl₂ (13.0 g, 15.9 mmol, 0.20 eq) was added. The mixture was stirred at 100° C. for 12 hrs. The mixture was filtered with diatomite and dilutered with H₂O (500 mL) and extracted with ethyl acetate (800 mL×3). The organic phase was washed with brine (800 mL×3), dried over Na₂SO₄, filtered and concentrated under reduced pressure to give a residue. The residue was purified by column chromatography (SiO₂, petroleum ether/ethyl acetate=100/1 to 1/1, R_{f}=0.30) to give the title compound (18.0 g, 83% yield) as a white solid. ¹H NMR (DMSO-d₆) δ 7.22, 7.21 (2s, 1H each), 3.75 (s, 3H), 2.27 (s, 3H), 1.30 (s, 12H).

### Step D′: (4-Cyano-2-hydroxy-6-methyl-phenyl)boronic acid

A solution of aforementioned 3-methoxy-5-methyl-4-(4,4,5,5-tetramethyl-1,3,2-dioxaborolan-2-yl)benzonitrile (17.0 g, 96.0 mmol, 1.00 eq) in dichloromethane (170 mL) was cooled to 0° C. and BBr₃ (38.9 g, 155 mmol, 2.50 eq) was added dropwise at 0° C. The mixture was stirred at 0° C. for 0.5 hr. The mixture was poured into H₂O (200 mL), filtered, and the cake was collected and triturated with EtOAc (20 mL) to give the title compound (4.67 g, 42% yield) as a gray solid. LCMS: m/z 178.1 [M+H]⁺, ESI pos.

### Step B: 3-Hydroxy-5-methyl-4-[2-(6-oxo-3,5-dihydro-1H-pyrrolo[3,4-c]pyridin-2-yl)oxazolo[4,5-b]pyridin-5-yl]benzonitrile

A mixture of 2-(5-bromooxazolo[4,5-b]pyridin-2-yl)-3,5-dihydro-1H-pyrrolo[3,4-c]pyridin-6-one (Example 3, step A) (35 mg, 0.09 mmol, 1.00 eq, 90% purity), (4-cyano-2-hydroxy-6-methyl-phenyl)boronic acid (step D′) (9 mg, 0.16 mmol, 1.73 eq), cesium carbonate (93 mg, 0.29 mmol, 3.02 eq) and XPhos Pd G3 (13 mg, 0.02 mmol, 0.16 eq) in 1,4-dioxane (1.0 mL) and water (0.25 mL) was flushed with argon and stirred at 95° C. for 2 hours. The reaction mixture was cooled to room temperature and extracted with a mixture of dichloromethane/methanol (9:1) and half-saturated aq. NH₄Cl-solution. The aqueous layers were backextracted twice with a mixture of dichloromethane/methanol (9:1). The combined organic layers were dried over sodium sulfate, filtered and concentrated in vacuo. The crude product was adsorbed on ISOLUTE HM-N and purified by flash chromatography (silica gel, gradient 0% to 20% methanol in dichloromethane). All fractions containing product were combined and concentrated in vacuo. The residue was triturated with ethyl acetate to afford the title compound (7.7 mg, 20% yield) as an off-white powder. LCMS: m/z 386.1 [M+H]⁺, ESI pos.

### Example 4

### 2-[5-(4-Chloro-2-hydroxy-6-methyl-phenyl)oxazolo[4,5-b]pyridin-2-yl]-3,5-dihydro-1H-pyrrolo[3,4-c]pyridin-6-one

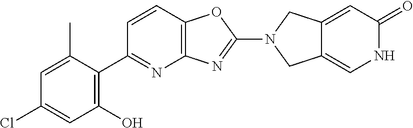 [SMILES: Cc1cc(Cl)cc(O)c1-c1ccc2oc(N3Cc4c[nH]c(=O)cc4C3)nc2n1]

A mixture of 2-(5-bromooxazolo[4,5-b]pyridin-2-yl)-3,5-dihydro-1H-pyrrolo[3,4-c]pyridin-6-one (Example 3, step A) (37 mg, 0.10 mmol, 1.00 eq, 90% purity), (4-chloro-2-hydroxy-6-methyl-phenyl)boronic acid (CAS #1207961-50-9, 30 mg, 0.16 mmol, 1.61 eq), potassium carbonate (50 mg, 0.36 mmol, 3.62 eq) and 1,1′-bis(diphenylphosphino)ferrocene-palladium(II) dichloride dichloromethane complex (13 mg, 0.02 mmol, 0.16 eq) in 1,4-dioxane (0.80 mL) and water (0.40 mL) was flushed with argon and stirred at 95° C. for 2 hours. The reaction mixture was cooled to room temperature and extracted with a mixture of dichloromethane/methanol (19:1) and half-saturated aq. NH₄Cl-solution. The organic layer was washed with water and brine. The aqueous layers were back extracted twice with a mixture of dichloromethane/methanol (19:1). The combined organic layers were dried over sodium sulfate, filtered and concentrated in vacuo. The crude product was adsorbed on ISOLUTE HM-N and purified by flash chromatography (silica gel, 4 g, gradient 0% to 100% (dichloromethane:methanol:NH₄OH 9:1:0.05) in dichloromethane). All fractions containing product were combined and concentrated in vacuo. The residue was triturated with ethyl acetate to afford the title compound (18 mg, 43% yield) as a light brown powder. LCMS: m/z 395.1 [M+H]⁺, ESI pos.

### Example 5

### 5-(2-Hydroxyethyl)-2-[5-[2-hydroxy-6-methyl-4-(trifluoromethyl)phenyl]oxazolo[4,5-b]pyridin-2-yl]-1,3-dihydropyrrolo[3,4-c]pyridin-6-one

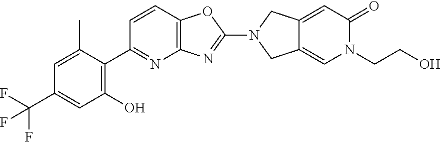 [SMILES: Cc1cc(C(F)(F)F)cc(O)c1-c1ccc2oc(N3Cc4cc(=O)n(CCO)cc4C3)nc2n1]

### Step A: tert-Butyl 5-(2-hydroxyethyl)-6-oxo-1,3-dihydropyrrolo[3,4-c]pyridine-2-carboxylate

To a suspension of tert-butyl 6-oxo-3,5-dihydro-1H-pyrrolo[3,4-c]pyridine-2-carboxylate (Example 1, step B) (100 mg, 0.38 mmol, 1.00 eq, 90% purity) in N,N-dimethylformamide (0.50 mL) was added cesium carbonate (290 mg, 0.89 mmol, 2.34 eq) followed by 2-iodoethanol (132 mg, 0.06 mL, 0.77 mmol, 2.02 eq). The reaction mixture was stirred at 90° C. for 16 hours and left standing at room temperature for 16 hours. The reaction mixture was extracted with 5% aq. LiCl-solution and ethyl acetate. The aqueous layer was backextracted with ethyl acetate. The organic layers were washed three times with 5% aq. LiCl-solution and once with brine. The combined organic layers were dried over sodium sulfate, filtered and concentrated in vacuo to afford the title compound (120 mg, 96% yield, 85% purity) as an off-white solid, which was used without further purification. LCMS: m/z 281.1 [M+H]⁺, ESI pos.

### Step B: 2-(5-Bromooxazolo[4,5-b]pyridin-2-yl)-5-(2-hydroxyethyl)-1,3-dihydropyrrolo[3,4-c]pyridin-6-one

To a solution of tert-butyl 5-(2-hydroxyethyl)-6-oxo-1,3-dihydropyrrolo[3,4-c]pyridine-2-carboxylate (Example 5, step A) (117 mg, 0.35 mmol, 1.14 eq, 85% purity) in dichloromethane (0.92 mL) and methanol (0.46 mL) was added dropwise 4 M HCl in dioxane (1.03 g, 0.86 mL, 3.44 mmol, 11.1 eq). Let stir at room temperature for 2 hours. The reaction mixture was concentrated in vacuo. The residue was taken up in 1,4-dioxane (0.74 mL) and water (0.08 mL) and 5-bromo-2-(methylthio)oxazolo[4,5-b]pyridine (CAS #1780768-09-3, 80 mg, 0.31 mmol, 1.00 eq) was added followed by N,N-diisoproylethylamine (129 mg, 0.17 mL, 1.00 mmol, 3.22 eq). The reaction mixture was stirred at 100° C. for 16 hours. The reaction mixture was cooled to room temperature and then extracted with a mixture of dichloromethane/methanol (9:1) and half-saturated aq. NaHCO₃-solution. The aqueous layer was backextracted five times with a mixture of dichloromethane/methanol (9:1). The combined organic layers were dried over sodium sulfate, filtered and concentrated in vacuo. The residue was adsorbed on ISOLUTE HM-N and purified by flash chromatography (silica gel, 12 g, gradient 0% to 10% methanol in dichloromethane) to afford the title compound (54 mg, 42% yield, 90% purity) as an off-white solid. LCMS: m/z 377.0/379.0 [M+H]⁺, ESI pos.

### Step C: 5-(2-Hydroxyethyl)-2-[5-[2-hydroxy-6-methyl-4-(trifluoromethyl)phenyl]oxazolo[4,5-b]pyridin-2-yl]-1,3-dihydropyrrolo[3,4-c]pyridin-6-one

A mixture of 2-(5-bromooxazolo[4,5-b]pyridin-2-yl)-5-(2-hydroxyethyl)-1,3-dihydropyrrolo[3,4-c]pyridin-6-one (Example 5, step B) (53 mg, 0.13 mmol, 1.00 eq, 90% purity), 3-methyl-2-(4,4,5,5-tetramethyl-1,3,2-dioxaborolan-2-yl)-5-(trifluoromethyl)phenol (CAS #2557358-38-8, 73 mg, 0.22 mmol, 1.72 eq, 90% purity), cesium carbonate (125 mg, 0.38 mmol, 3.03 eq) and XPhos Pd G3 (17 mg, 0.02 mmol, 0.16 eq) in 1,4-dioxane (1.2 mL) and water (0.30 mL) was flushed with argon and stirred at 95° C. for 2 hours. The reaction mixture was cooled to room temperature and extracted with a mixture of dichloromethane/methanol (9:1) and half-saturated aq. NH₄Cl-solution. The aqueous layers were backextracted twice with a mixture of dichloromethane/methanol (9:1). The organic layers were washed with water and brine (a precipitate was present in the aqueous layers). The combined aqueous layers were filtered, rinsing with water and ethyl acetate. The filter cake was dried on the rotavap and then put under high vacuum to afford the title compound (32 mg, 51% yield) as an off-white powder. LCMS: m/z 473.1 [M+H]⁺, ESI pos.

### Examples 6-9

5-(4-Chloro-2-hydroxy-6-methyl-phenyl)-3H-oxazolo[4,5-b]pyridine-2-thione Intermediate 1 (1320.0 mg, 4.51 mmol, 1.0 eq) was dissolved in DCM (50 mL) and oxalyl chloride (5.8 mL, 67.6 mmol, 15.0 eq) was added, followed by drop-wise addition of DMF (1.5 mL). The mixture was stirred at room temperature for 30 min, then concentrated in vacuo. The resulting residue was taken up in DCM (45 mL) and sat. aq. K₂CO₃ (15 mL) was added. The DCM layer was isolated and the aqueous back-extracted with DCM (30 mL). The combined organic extracts were dried using a phase separator. The resulting solution (containing 5-chloro-2-(2-chlorooxazolo[4,5-b]pyridin-5-yl)-3-methylphenol) was made up to 92 mL with DCM and used as a stock solution in a series of reactions using the following general procedure:

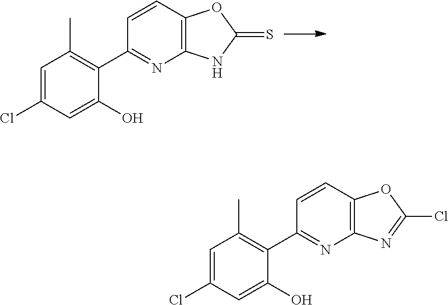 [SMILES: Cc1cc(Cl)cc(O)c1-c1ccc2oc(=S)[nH]c2n1 ; Cc1cc(Cl)cc(O)c1-c1ccc2oc(Cl)nc2n1]

Amine (5.41 mmol, 1.2 eq) was dissolved in DMF (0.1 mL) and triethylamine (0.02 mL, 0.15 mmol, 3.0 eq) was added, followed by 1 mL of the stock solution of 5-chloro-2-(2-chlorooxazolo[4,5-b]pyridin-5-yl)-3-methylphenol (1 eq.). The mixture was shaken at room temperature for 20 min. The DCM was evaporated overnight, and the resulting residue made up to 1 mL with DMF, and then filtered. The resulting filtrate was purified by prep HPLC.

| Ex- | | | Amine name, |
|---|---|---|---|
| ample | | | Prep Gradient, |
| No | Structure | Name | LCMS data |
| 6 | 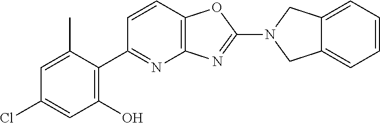 [SMILES: Cc1cc(Cl)cc(O)c1-c1ccc2oc(N3Cc4ccccc4C3)nc2n1] | 5-Chloro-2-(2- isoindolin-2- yloxazolo[4,5- b]pyridin-5-yl)-3- methyl-phenol | isoindoline (CAS #496-12-8), 40-70% MeCN in water, LCMS m/z 378.0 (M + H)+, ESI pos. |
| 7 | 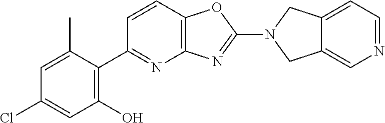 [SMILES: Cc1cc(Cl)cc(O)c1-c1ccc2oc(N3Cc4ccncc4C3)nc2n1] | 5-chloro-2-[2-(1,3- dihydropyrrolo[3,4- c]pyridin-2- yl)oxazolo[4,5- b]pyridin-5-yl]-3- methyl-phenol | 2,3-Dihydro-1H- pyrrolo[3,4- c]pyridine dihydrochloride (CAS # 6000-50- 6). 15-45% MeCN in water, LCMS m/z 378.91 [M + H]+, ESI pos. |
| 8 | 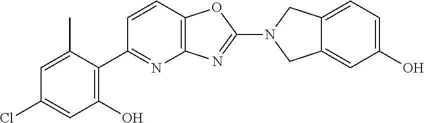 [SMILES: Cc1cc(Cl)cc(O)c1-c1ccc2oc(N3Cc4ccc(O)cc4C3)nc2n1] | 2-[5-(4-Chloro-2- hydroxy-6-methyl- phenyl)oxazolo[4,5- b]pyridin-2- yl]isoindolin-5-ol | 2,3-dihydro-1H- isoindol-5-ol hydrobromide (CAS # 105358- 58-5), 30-60% MeCN in water, LCMS m/z 394.0 (M + H)+, ESI pos. |
| 9 | 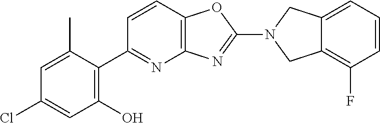 [SMILES: Cc1cc(Cl)cc(O)c1-c1ccc2oc(N3Cc4cccc(F)c4C3)nc2n1] | 5-Chloro-2-[2-(4- fluoroisoindolin-2- yl)oxazolo[4,5- b]pyridin-5-yl]-3- methyl-phenol | 4- Fluoroisoindoline hydrochloride (CAS # 924305- 06-6), 40-70% MeCN in water, LCMS m/z 395.9 (M + H)+, ESI pos. |

### Example 10

### 4-[2-(1,3-Dihydropyrrolo[3,4-c]pyridin-2-yl)oxazolo[4,5-b]pyridin-5-yl]-3-hydroxy-5-methyl-benzonitrile

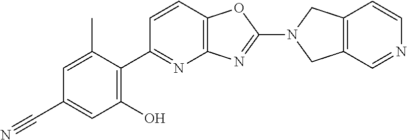 [SMILES: Cc1cc(C#N)cc(O)c1-c1ccc2oc(N3Cc4ccncc4C3)nc2n1]

### Step A: 5-Chloro-2-(1,3-dihydropyrrolo[3,4-c]pyridin-2-yl)oxazolo[4,5-b]pyridine

To a mixture of 5-chloro-2-(methylthio)oxazolo[4,5-b]pyridine (CAS #1783370-92-2, 100 mg, 0.47 mmol, 1.00 eq) and 2,3-dihydro-1H-pyrrolo[3,4-c]pyridine dihydrochloride (CAS #6000-50-6, 128 mg, 0.66 mmol, 1.40 eq) in 1,4-dioxane (0.90 mL) and water (0.10 mL) was added N,N-diisopropylethylamine (243 mg, 0.32 mL, 1.88 mmol, 3.97 eq). The reaction mixture was stirred at 90° C. for 72 hours. The reaction mixture was cooled to room temperature and extracted with half-saturated aq. NaHCO₃-solution and three times with a mixture of dichloromethane/methanol (19:1). The combined organic layers were dried over sodium sulfate, filtered and concentrated in vacuo. The crude product was adsorbed on ISOLUTE HM and purified by flash chromatography (silica gel, 12 g, gradient 0% to 5% methanol in dichloromethane) to afford the title compound (117 mg, 86% yield) as an off-white solid. LCMS: m/z 273.1 [M+H]⁺, ESI pos.

### Step B: 4-[2-(1,3-Dihydropyrrolo[3,4-c]pyridin-2-yl)oxazolo[4,5-b]pyridin-5-yl]-3-hydroxy-5-methyl-benzonitrile

The title compound was obtained as an off-white solid, LCMS: m/z 370.1 [M+H]⁺, ESI pos, using chemistry similar to that described in Example 3, step B starting from 5-chloro-2-(1,3-dihydropyrrolo[3,4-c]pyridin-2-yl)oxazolo[4,5-b]pyridine (Example 10, step A) and (4-cyano-2-hydroxy-6-methyl-phenyl)boronic acid (Example 3, step D′).

### Example 11

### 5-Chloro-3-methyl-2-[2-(2-methyl-7,8-dihydro-5H-pyrido[4,3-d]pyrimidin-6-yl)oxazolo[4,5-b]pyridin-5-yl]phenol

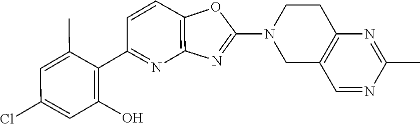 [SMILES: Cc1ncc2c(n1)CCN(c1nc3nc(-c4c(C)cc(Cl)cc4O)ccc3o1)C2]

### Step A: 5-Chloro-2-(2-methyl-7,8-dihydro-5H-pyrido[4,3-d]pyrimidin-6-yl)oxazolo[4,5-b]pyridine

To a mixture of 5-chloro-2-(methylthio)oxazolo[4,5-b]pyridine (CAS #1783370-92-2, 80 mg, 0.38 mmol, 1.00 eq) and 2-methyl-5,6,7,8-tetrahydropyrido[4,3-d]pyrimidine (CAS #676994-65-3, 74 mg, 0.50 mmol, 1.31 eq) in 1,4-dioxane (0.90 mL) and water (0.10 mL) was added N,N-diisoproylethylamine (106 mg, 0.14 mL, 0.82 mmol, 2.17 eq). The reaction mixture was stirred at 110° C. for 16 hours. The reaction mixture was cooled to room temperature and extracted with half-saturated aq. NaHCO₃-solution and three times with a mixture of dichloromethane/methanol (19:1). The combined organic layers were dried over sodium sulfate, filtered and concentrated in vacuo. The crude product was adsorbed on ISOLUTE HM-N and purified by flash chromatography (silica gel, gradient 0% to 5% methanol in dichloromethane) to afford the title compound (99 mg, 82% yield) as an off-white solid. LCMS: m/z 302.1 [M+H]⁺, ESI pos.

### Step B: 5-Chloro-3-methyl-2-[2-(2-methyl-7,8-dihydro-5H-pyrido[4,3-d]pyrimidin-6-yl)oxazolo[4,5-b]pyridin-5-yl]phenol

The title compound was obtained as an off-white foam, LCMS: m/z 408.2 [M+H]⁺, ESI pos, using chemistry similar to that described in Example 4 starting from 5-chloro-2-(2-methyl-7,8-dihydro-5H-pyrido[4,3-d]pyrimidin-6-yl)oxazolo[4,5-b]pyridine (Example 11, step A) and (4-chloro-2-hydroxy-6-methyl-phenyl)boronic acid (CAS #1207961-50-9).

### Example 12

### 2-[2-(6-Methoxy-1,3-dihydropyrrolo[3,4-c]pyridin-2-yl)oxazolo[4,5-b]pyridin-5-yl]-3-methyl-5-(trifluoromethyl)phenol

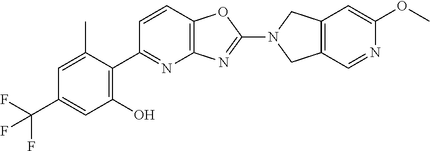 [SMILES: COc1cc2c(cn1)CN(c1nc3nc(-c4c(C)cc(C(F)(F)F)cc4O)ccc3o1)C2]

### Step A: 6-Methoxy-2,3-dihydro-1H-pyrrolo[3,4-c]pyridine

To a mixture of 6-chloro-2,3-dihydro-1H-pyrrolo[3,4-c]pyridine (CAS #905273-90-7, 210 mg, 1.36 mmol, 1.00 eq) in 1,4-dioxane (2.8 mL) was added sodium methoxide solution (25 wt. % in methanol) (1.42 g, 1.5 mL, 6.56 mmol, 4.83 eq) at room temperature. The reaction mixture was stirred at 100° C. for 16 hours. The reaction mixture was cooled to room temperature, poured onto half-saturated aq. NH₄Cl-solution and extracted three times with dichloromethane. The combined organic layers were dried over sodium sulfate, filtered and concentrated in vacuo.

The crude product was adsorbed on ISOLUTE HM-N and purified by flash chromatography (silica gel, gradient 0% to 20% methanol in dichloromethane) to afford the title compound (175 mg, 81% yield) as a purple solid. LCMS: m/z 151.1 [M+H]⁺, ESI pos.

### Step B: 5-Chloro-2-(6-methoxy-1,3-dihydropyrrolo[3,4-c]pyridin-2-yl)oxazolo[4,5-b]pyridine

To a mixture of 5-chloro-2-(methylthio)oxazolo[4,5-b]pyridine (CAS #1783370-92-2, 115 mg, 0.54 mmol, 1.00 eq) and 6-methoxy-2,3-dihydro-1H-pyrrolo[3,4-c]pyridine (Example 12, step A) (114 mg, 0.72 mmol, 1.32 eq) in 1,4-dioxane (1.3 mL) and water (0.14 mL) was added N,N-diisoproylethylamine (152 mg, 0.20 mL, 1.18 mmol, 2.16 eq). The reaction mixture was stirred at 100° C. for 16 hours. The reaction mixture was cooled to room temperature and extracted with half-saturated aq. NaHCO₃-solution and three times with a mixture of dichloromethane/methanol (19:1). The combined organic layers were dried over sodium sulfate, filtered and concentrated in vacuo. The crude product was adsorbed on ISOLUTE HM-N and purified by flash chromatography (silica gel, 12 g, gradient 0% to 5% methanol in dichloromethane) to afford the title compound (159 mg, 92% yield) as a pink solid. LCMS: m/z 303.1 [M+H]⁺, ESI pos.

### Step C: 2-[2-(6-Methoxy-1,3-dihydropyrrolo[3,4-c]pyridin-2-yl)oxazolo[4,5-b]pyridin-5-yl]-3-methyl-5-(trifluoromethyl)phenol

The title compound was obtained as a light brown solid, LCMS: m/z 443.2 [M+H]⁺, ESI pos, using chemistry similar to that described in Example 1, step D starting from 5-chloro-2-(6-methoxy-1,3-dihydropyrrolo[3,4-c]pyridin-2-yl)oxazolo[4,5-b]pyridine (Example 12, step B) and 3-methyl-2-(4,4,5,5-tetramethyl-1,3,2-dioxaborolan-2-yl)-5-(trifluoromethyl)phenol (CAS #2557358-38-8).

### Example 13

### 5-Chloro-2-[2-(5,7-dihydropyrrolo[3,4-d]pyrimidin-6-yl)oxazolo[4,5-b]pyridin-5-yl]-3-methyl-phenol formic acid salt

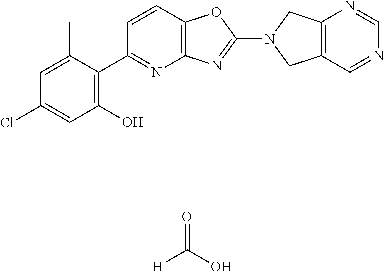 [SMILES: Cc1cc(Cl)cc(O)c1-c1ccc2oc(N3Cc4cncnc4C3)nc2n1 ; O=CO]

### Step A: 5-Chloro-2-(5,7-dihydropyrrolo[3,4-d]pyrimidin-6-yl)oxazolo[4,5-b]pyridine

To a mixture of 5-chloro-2-(methylthio)oxazolo[4,5-b]pyridine (CAS #1783370-92-2, 200 mg, 1.00 mmol, 1.00 eq) and 6,7-dihydro-5H-pyrrolo[3,4-d]pyrimidine (CAS #53493-80-4, 133 mg, 1.10 mmol, 1.10 eq) in 1,4-dioxane (2.1 mL) was added under argon triethylamine (113 mg, 0.16 mL, 1.12 mmol, 1.12 eq). The reaction mixture was stirred at 90° C. for 16 hours. Additional 6,7-dihydro-5H-pyrrolo[3,4-d]pyrimidine (CAS #53493-80-4, 121 mg, 1.00 mmol, 1.00 eq) was added and the reaction mixture was stirred at 100° C. for 16 hours. The reaction mixture was cooled to room temperature and extracted with ethyl acetate and half-saturated aq. NaHCO₃-solution. The organic layers were washed with water and brine. The combined organic layers were dried over sodium sulfate, filtered and concentrated in vacuo. The crude product was adsorbed on ISOLUTE HM-N and purified by flash chromatography (silica gel, gradient 0% to 80% ethyl acetate in heptane) to afford the title compound (70 mg, 26% yield) as a brown solid. LCMS: m/z 274.0 [M+H]⁺, ESI pos.

### Step B: 5-Chloro-2-[2-(5,7-dihydropyrrolo[3,4-d]pyrimidin-6-yl)oxazolo[4,5-b]pyridin-5-yl]-3-methyl-phenol formic acid salt

The title compound was obtained as a white solid, LCMS: m/z 380.1 [M+H]⁺, ESI pos, using chemistry similar to that described in Example 4 starting from 5-chloro-2-(5,7-dihydropyrrolo[3,4-d]pyrimidin-6-yl)oxazolo[4,5-b]pyridine (Example 13, step A) and (4-chloro-2-hydroxy-6-methyl-phenyl)boronic acid (CAS #1207961-50-9) and purification by reversed-phase chromatography (column: YMC-Triart C18, eluent: water+0.1% HCOOH in acetonitrile).

### Example 14

### 5-Chloro-2-[2-(4,6-dihydro-2H-pyrrolo[3,4-c]pyrazol-5-yl)oxazolo[4,5-b]pyridin-5-yl]-3-methyl-phenol

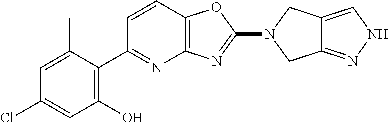

### Step A: 5-Chloro-2-(4,6-dihydro-2H-pyrrolo[3,4-c]pyrazol-5-yl)oxazolo[4,5-b]pyridine

To a mixture of 5-chloro-2-(methylthio)oxazolo[4,5-b]pyridine (CAS #1783370-92-2, 100 mg, 0.47 mmol, 1.00 eq) and 2,4,5,6-tetrahydropyrrolo[3,4-c]pyrazole (67 mg, 0.61 mmol, 1.30 eq) in 1,4-dioxane (1.0 mL) and water (0.11 mL) was added N,N-diisoproylethylamine (135 mg, 0.178 mL, 1.05 mmol, 2.21 eq). The reaction mixture was stirred at 90° C. for 72 hours and at 110° C. for 16 hours. The reaction mixture was cooled to room temperature and extracted with half-saturated aq. NaHCO₃-solution and three times with a mixture of dichloromethane/methanol (19:1) and four times with a mixture of dichloromethane/methanol (9:1). The combined organic layers were dried over sodium sulfate, filtered and concentrated in vacuo. The crude product was adsorbed on ISOLUTE HM and purified by flash chromatography (silica gel, gradient 0% to 5% methanol in dichloromethane) to afford the title compound (77 mg, 56% yield, 90% purity) as a white solid. LCMS: m/z 262.1 [M+H]⁺, ESI pos.

### Step B: 5-Chloro-2-[2-(4,6-dihydro-2H-pyrrolo[3,4-c]pyrazol-5-yl)oxazolo[4,5-b]pyridin-5-yl]-3-methyl-phenol

The title compound was obtained as an off-white solid, LCMS: m/z 368.2 [M+H]⁺, ESI pos, using chemistry similar to that described in Example 4 starting from 5-chloro-2-(4,6-dihydro-2H-pyrrolo[3,4-c]pyrazol-5-yl)oxazolo[4,5-b]pyridine (Example 14, step A) and (4-chloro-2-hydroxy-6-methyl-phenyl)boronic acid (CAS #1207961-50-9).

### Example 15

### 5-Chloro-2-[2-(3,4-dihydro-1H-2,7-naphthyridin-2-yl)oxazolo[4,5-b]pyridin-5-yl]-3-methyl-phenol

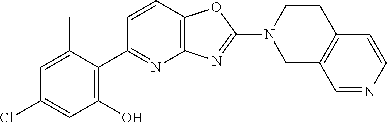 [SMILES: Cc1cc(Cl)cc(O)c1-c1ccc2oc(N3CCc4ccncc4C3)nc2n1]

### Step A: 5-Chloro-2-(3,4-dihydro-1H-2,7-naphthyridin-2-yl)oxazolo[4,5-b]pyridine

To a mixture of 5-chloro-2-(methylthio)oxazolo[4,5-b]pyridine (CAS #1783370-92-2, 100 mg, 0.47 mmol, 1.00 eq) and 1,2,3,4-tetrahydro-2,7-naphthyridine hydrochloride (CAS #1354940-72-9, 113 mg, 0.66 mmol, 1.40 eq) in 1,4-dioxane (0.90 mL) and water (0.10 mL) was added N,N-diisoproylethylamine (243 mg, 0.32 mL, 1.88 mmol, 3.97 eq). The reaction mixture was stirred at 110° C. for 16 hours. The reaction mixture was cooled to room temperature and extracted with half-saturated aq. NaHCO₃-solution and three times with a mixture of dichloromethane/methanol (19:1). The combined organic layers were dried over sodium sulfate, filtered and concentrated in vacuo. The crude product was adsorbed on ISOLUTE HM-N and purified by flash chromatography (silica gel, gradient 0% to 5% methanol in dichloromethane) to afford the title compound (116 mg, 81% yield) as a light yellow solid. LCMS: m/z 287.1 [M+H]⁺, ESI pos.

### Step B: 5-Chloro-2-[2-(3,4-dihydro-1H-2,7-naphthyridin-2-yl)oxazolo[4,5-b]pyridin-5-yl]-3-methyl-phenol

The title compound was obtained as a white solid, LCMS: m/z 393.2 [M+H]⁺, ESI pos, using chemistry similar to that described in Example 4 starting from 5-chloro-2-(3,4-dihydro-1H-2,7-naphthyridin-2-yl)oxazolo[4,5-b]pyridine (Example 15, step A) and (4-chloro-2-hydroxy-6-methyl-phenyl)boronic acid (CAS #1207961-50-9).

### Example 16

### 3-Hydroxy-4-(2-isoindolin-2-yloxazolo[4,5-b]pyridin-5-yl)-5-methyl-benzonitrile

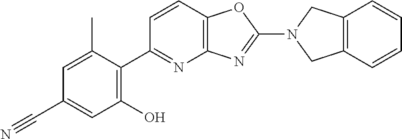 [SMILES: Cc1cc(C#N)cc(O)c1-c1ccc2oc(N3Cc4ccccc4C3)nc2n1]

### Step A: 5-Chloro-2-isoindolin-2-yl-oxazolo[4,5-b]pyridine

To a mixture of 5-chloro-2-(methylthio)oxazolo[4,5-b]pyridine (CAS #1783370-92-2, 150 mg, 0.74 mmol, 1.00 eq) and isoindoline (CAS #496-12-8, 121 mg, 0.115 mL, 0.96 mmol, 1.30 eq) in 1,4-dioxane (1.8 mL) and water (0.20 mL) was added N,N-diisoproylethylamine (143 mg, 0.189 mL, 1.11 mmol, 1.50 eq). The reaction mixture was stirred at 100° C. for 16 hours in a sealed tube. The reaction mixture was extracted with ethyl acetate and 1 M Na₂CO₃-solution. The organic layers were washed with water and brine. The combined organic layers were dried over sodium sulfate, filtered and concentrated in vacuo. The residue was purified by flash chromatography (silica gel, 12 g, gradient 0% to 20% ethyl acetate in dichloromethane) to afford the title compound (151 mg, 71% yield) as a light grey solid. LCMS: m/z 272.0 [M+H]⁺, ESI pos.

### Step B: 3-Hydroxy-4-(2-isoindolin-2-yloxazolo[4,5-b]pyridin-5-yl)-5-methyl-benzonitrile

The title compound was obtained as an off-white solid, LCMS: m/z 369.2 [M+H]⁺, ESI pos, using chemistry similar to that described in Example 3, step B starting from 5-chloro-2-isoindolin-2-yl-oxazolo[4,5-b]pyridine (Example 16, step A) and (4-cyano-2-hydroxy-6-methyl-phenyl)boronic acid (Example 3, step D′).

### Example 17

### 4-[2-(7,8-Dihydro-5H-pyrido[4,3-d]pyrimidin-6-yl)oxazolo[4,5-b]pyridin-5-yl]-3-hydroxy-5-methyl-benzonitrile

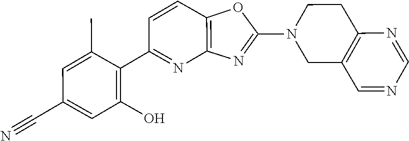 [SMILES: Cc1cc(C#N)cc(O)c1-c1ccc2oc(N3CCc4ncncc4C3)nc2n1]

### Step A: 5-Chloro-2-(7,8-dihydro-5H-pyrido[4,3-d]pyrimidin-6-yl)oxazolo[4,5-b]pyridine

To a mixture of 5-chloro-2-(methylthio)oxazolo[4,5-b]pyridine (CAS #1783370-92-2, 253 mg, 1.20 mmol, 1.00 eq) and 5,6,7,8-tetrahydropyrido[4,3-d]pyrimidine (CAS #192869-50-4, 227 mg, 1.68 mmol, 1.40 eq) in 1,4-dioxane (3.0 mL) was added under argon triethylamine (218 mg, 0.30 mL, 2.16 mmol, 1.80 eq). The reaction mixture was stirred at 90° C. for 48 hours. The reaction mixture was cooled to room temperature and extracted with ethyl acetate and aq. NaHCO₃-solution. The organic layers were washed with water and brine. The combined organic layers were dried over sodium sulfate, filtered and concentrated in vacuo. The residue was purified by flash chromatography (silica gel, gradient 0% to 10% methanol in dichloromethane) to afford the title compound (270 mg, 74% yield) as light yellow solid. LCMS: m/z 288.0 [M+H]⁺, ESI pos.

### Step B: 4-[2-(7,8-Dihydro-5H-pyrido[4,3-d]pyrimidin-6-yl)oxazolo[4,5-b]pyridin-5-yl]-3-hydroxy-5-methyl-benzonitrile

The title compound was obtained as a white solid, LCMS: m/z 385.1 [M+H]⁺, ESI pos, using chemistry similar to that described in Example 3, step B starting from 5-chloro-2-(7,8-dihydro-5H-pyrido[4,3-d]pyrimidin-6-yl)oxazolo[4,5-b]pyridine (Example 17, step A) and (4-cyano-2-hydroxy-6-methyl-phenyl)boronic acid (Example 3, step D′).

### Example 18

### 2-[2-(5,7-Dihydropyrrolo[3,4-d]pyrimidin-6-yl)oxazolo[4,5-b]pyridin-5-yl]-3-methyl-5-(trifluoromethyl)phenol

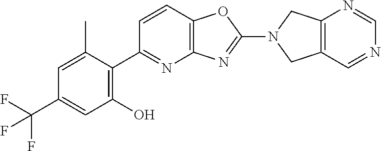 [SMILES: Cc1cc(C(F)(F)F)cc(O)c1-c1ccc2oc(N3Cc4cncnc4C3)nc2n1]

The title compound was obtained as an off-white solid, LCMS: m/z 414.1 [M+H]⁺, ESI pos, using chemistry similar to that described in Example 1, step D starting from 5-chloro-2-(5,7-dihydropyrrolo[3,4-d]pyrimidin-6-yl)oxazolo[4,5-b]pyridine (Example 13, step A) and 3-methyl-2-(4,4,5,5-tetramethyl-1,3,2-dioxaborolan-2-yl)-5-(trifluoromethyl)phenol (CAS #2557358-38-8).

### Example 19

### 7-[5-[2-Hydroxy-6-methyl-4-(trifluoromethyl)phenyl]oxazolo[4,5-b]pyridin-2-yl]-2,5,6,8-tetrahydro-2,7-naphthyridin-3-one

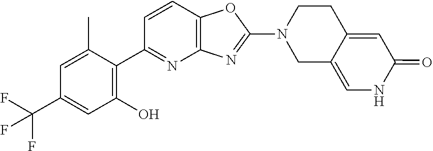 [SMILES: Cc1cc(C(F)(F)F)cc(O)c1-c1ccc2oc(N3CCc4cc(=O)[nH]cc4C3)nc2n1]

### Step A: tert-Butyl 6-benzyloxy-3,4-dihydro-1H-2,7-naphthyridine-2-carboxylate

To a mixture of tert-butyl 6-chloro-3,4-dihydro-1H-2,7-naphthyridine-2-carboxylate (CAS #1396777-92-6, 150 mg, 0.56 mmol, 1.00 eq) and benzyl alcohol (91 mg, 0.087 mL, 0.84 mmol, 1.50 eq) in 1,4-dioxane (2.0 mL) was added potassium tert-butoxide (88 mg, 0.78 mmol, 1.40 eq). The reaction mixture was stirred at 100° C. for 16 hours. The reaction mixture was cooled to room temperature. The reaction mixture was quenched with water and extracted twice with ethyl acetate. The organic layers were washed with brine. The combined organic layers were dried over sodium sulfate, filtered and concentrated in vacuo. The residue was purified by flash chromatography (silica gel, 12 g, gradient 0% to 30% ethyl acetate in heptane) to afford the title compound (80 mg, 40% yield) as a colorless oil. LCMS: m/z 341.2 [M+H]⁺, ESI pos.

### Step B: 6-Benzyloxy-1,2,3,4-tetrahydro-2,7-naphthyridine hydrochloride

To a solution of tert-butyl 6-benzyloxy-3,4-dihydro-1H-2,7-naphthyridine-2-carboxylate (Example 19, step A) (78 mg, 0.22 mmol, 1.00 eq) in dichloromethane (2.0 mL) and methanol (1.0 mL) was added at room temperature 4 M HCl in 1,4-dioxane (0.54 mL, 2.18 mmol, 10.00 eq) dropwise. The reaction mixture was stirred at room temperature for 16 hours. The white suspension was concentrated in vacuo to afford the title compound (53 mg, 84% yield) as a white solid. LCMS: m/z 241.1 [M+H]⁺, ESI pos.

### Step C: 2-(6-Benzyloxy-3,4-dihydro-1H-2,7-naphthyridin-2-yl)-5-chloro-oxazolo[4,5-b]pyridine

To a mixture of 5-chloro-2-(methylthio)oxazolo[4,5-b]pyridine (CAS #1783370-92-2, 32 mg, 0.16 mmol, 1.00 eq) and 6-benzyloxy-1,2,3,4-tetrahydro-2,7-naphthyridine hydrochloride (Example 19, step B) (51 mg, 0.18 mmol, 1.10 eq) in 1,4-dioxane (1.0 mL) and water (0.10 mL) was added under argon triethylamine (48 mg, 0.067 mL, 0.48 mmol, 3.00 eq). The reaction mixture was stirred at 100° C. for 48 hours. The reaction mixture was cooled to room temperature and extracted with ethyl acetate and aq. NaHCO₃-solution. The organic layers were washed with water and brine. The combined organic layers were dried over sodium sulfate, filtered and concentrated in vacuo. The residue was purified by flash chromatography (silica gel, gradient 0% to 50% ethyl acetate in heptane) to afford the title compound (31 mg, 47% yield) as a light yellow foam. LCMS: m/z 393.1 [M+H]⁺, ESI pos.

### Step D: 7-[5-[2-Hydroxy-6-methyl-4-(trifluoromethyl)phenyl]oxazolo[4,5-b]pyridin-2-yl]-2,5,6,8-tetrahydro-2,7-naphthyridin-3-one

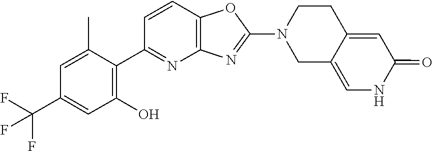 [SMILES: Cc1cc(C(F)(F)F)cc(O)c1-c1ccc2oc(N3CCc4cc(=O)[nH]cc4C3)nc2n1]

A mixture of 2-(6-benzyloxy-3,4-dihydro-1H-2,7-naphthyridin-2-yl)-5-chloro-oxazolo[4,5-b]pyridine (Example 19, step C) (30 mg, 0.07 mmol, 1.00 eq), 3-methyl-2-(4,4,5,5-tetramethyl-1,3,2-dioxaborolan-2-yl)-5-(trifluoromethyl)phenol (CAS #2557358-38-8, 35 mg, 0.12 mmol, 1.60 eq) and cesium carbonate (71 mg, 0.22 mmol, 3.00 eq) in 1,4-dioxane (1.6 mL) and water (0.40 mL) was put under argon, and XPhos Pd G3 (9 mg, 0.01 mmol, 0.15 eq) was added. The reaction mixture was stirred at 95° C. for 3 hours. Additional XPhos Pd G3 (9 mg, 0.01 mmol, 0.15 eq) was added and stirring was continued at 95° C. for 2 hours and at room temperature for 16 hours. The reaction mixture was cooled to room temperature, quenched with water and equal amount of saturated aq. NH₄Cl-solution and then extracted twice with ethyl acetate. The organic layers were washed with brine, dried over sodium sulfate, filtered and concentrated in vacuo. The crude product was adsorbed on ISOLUTE HM-N and purified by flash chromatography (silica gel, 12 g, gradient 0% to 50% (dichloromethane:methanol:NH₄OH 110:10:1) in dichloromethane) followed by crystallization with ethyl acetate/heptane 1:1 (v/v) to afford the title compound (9 mg, 27% yield) as a white solid. LCMS: m/z 443.1 [M+H]⁺, ESI pos.

### Example 20

### 6-[5-[2-Hydroxy-6-methyl-4-(trifluoromethyl)phenyl]oxazolo[4,5-b]pyridin-2-yl]-5,7-dihydro-1H-pyrrolo[3,4-b]pyridin-2-one

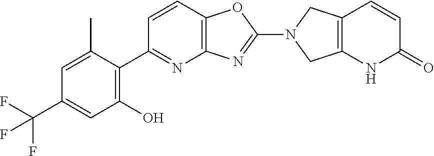 [SMILES: Cc1cc(C(F)(F)F)cc(O)c1-c1ccc2oc(N3Cc4ccc(=O)[nH]c4C3)nc2n1]

### Step A: tert-Butyl 2-benzyloxy-5,7-dihydropyrrolo[3,4-b]pyridine-6-carboxylate

To a mixture of tert-butyl 2-chloro-5,7-dihydropyrrolo[3,4-b]pyridine-6-carboxylate (CAS #1257854-60-6, 325 mg, 1.28 mmol, 1.00 eq) and benzyl alcohol (207 mg, 0.199 mL, 1.91 mmol, 1.50 eq) in 1,4-dioxane (5.0 mL) was added at room temperature potassium tert-butoxide (200 mg, 1.79 mmol, 1.40 eq). The reaction mixture was stirred at 100° C. for 16 hours. The reaction mixture was cooled to room temperature. The reaction mixture was quenched with water and extracted twice with ethyl acetate. The organic layers were washed with brine. The combined organic layers were dried over sodium sulfate, filtered and concentrated in vacuo. The residue was purified by flash chromatography (silica gel, 25 g, gradient 0% to 30% ethyl acetate in heptane) to afford the title compound (305 mg, 69% yield) as a purple oil. LCMS: m/z 327.2 [M+H]⁺, ESI pos.

### Step B: tert-Butyl 2-oxo-5,7-dihydro-1H-pyrrolo[3,4-b]pyridine-6-carboxylate

A solution of tert-butyl 2-benzyloxy-5,7-dihydropyrrolo[3,4-b]pyridine-6-carboxylate (Example 20, step A) (303 mg, 0.87 mmol, 1.00 eq) in ethyl acetate (10 mL) and methanol (10 mL) was three times alternating evacuated and flushed with argon. Palladium on activated charcoal, 10% Pd basis (93 mg, 0.09 mmol, 0.10 eq) was added carefully. The reaction flask was evacuated, flushed with argon, evacuated and flushed with hydrogen. The reaction mixture was stirred under hydrogen atmosphere (balloon) at room temperature for 4 hours. The reaction mixture was filtered and rinsed well with ethyl acetate/methanol. The filtrate was concentrated in vacuo to afford the title compound (208 mg, 96% yield) as a white solid, which was used without further purification. LCMS: m/z 237.1 [M+H]⁺, ESI pos.

### Step C: 1,5,6,7-Tetrahydropyrrolo[3,4-b]pyridin-2-one hydrochloride

To a solution of tert-butyl 2-oxo-5,7-dihydro-1H-pyrrolo[3,4-b]pyridine-6-carboxy late (Example 20, step B) (97 mg, 0.41 mmol, 1.00 eq) in dichloromethane (2.0 mL) and methanol (1.0 mL) was added dropwise at ambient temperature 4 M HCl in 1,4-dioxane (1.23 g, 1.03 mL, 4.11 mmol, 10.0 eq). The reaction mixture was stirred at room temperature for 16 hours. The reaction mixture was concentrated in vacuo to afford the title compound (87 mg, 98% yield) as a light grey solid. LCMS: m/z 137.1 [M+H]⁺, ESI pos.

### Step D: 6-(5-Chlorooxazolo[4,5-b]pyridin-2-yl)-5,7-dihydro-1H-pyrrolo[3,4-b]pyridin-2-one

To a mixture of 5-chloro-2-(methylthio)oxazolo[4,5-b]pyridine (CAS #1783370-92-2, 85 mg, 0.42 mmol, 1.00 eq) and 1,5,6,7-tetrahydropyrrolo[3,4-b]pyridin-2-one hydrochloride (Example 20, step C) (88 mg, 0.51 mmol, 1.20 eq) in 1,4-dioxane (1.0 mL) and water (0.10 mL) was added N,N-diisopropylethylamine (274 mg, 0.36 mL, 2.12 mmol, 5.00 eq). The reaction mixture was stirred in a sealed tube at 100° C. for 24 hours resulting in a grey suspension. The reaction mixture was cooled and diluted with a small amount of ethyl acetate, water and half-saturated aq. NaHCO₃-solution. The solid was filtered off, washed with a mixture of ethyl acetate/water followed by rinsing the solid with diethyl ether. The filter cake was put under high vacuum and dried at 50° C. for 1 hour to afford the title compound (67 mg, 52% yield) as a grey solid. LCMS: m/z 287.0 [M−H]⁻, ESI pos.

### Step E: 6-[5-[2-Hydroxy-6-methyl-4-(trifluoromethyl)phenyl]oxazolo[4,5-b]pyridin-2-yl]-5,7-dihydro-1H-pyrrolo[3,4-b]pyridin-2-one

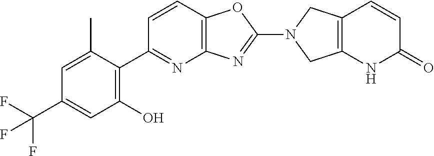 [SMILES: Cc1cc(C(F)(F)F)cc(O)c1-c1ccc2oc(N3Cc4ccc(=O)[nH]c4C3)nc2n1]

A mixture of 6-(5-chlorooxazolo[4,5-b]pyridin-2-yl)-5,7-dihydro-1H-pyrrolo[3,4-b]pyridin-2-one (Example 20, step D) (66 mg, 0.23 mmol, 1.00 eq), 3-methyl-2-(4,4,5,5-tetramethyl-1,3,2-dioxaborolan-2-yl)-5-(trifluoromethyl)phenol (CAS #2557358-38-8, 111 mg, 0.37 mmol, 1.60 eq) and cesium carbonate (223 mg, 0.69 mmol, 3.00 eq) in 1,4-dioxane (1.0 mL) and water (1.0 mL) and N,N-dimethylformamide (3.0 mL) was flushed with argon and XPhos Pd G3 (29 mg, 0.03 mmol, 0.15 eq) was added. The reaction mixture was stirred at 100° C. for 3 hours. The reaction mixture was cooled to room temperature, quenched with water and equal amount of saturated aq. NH₄Cl-solution. The mixture was extracted twice with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, 12 g, gradient 0% to 50% (dichloromethane:methanol:NH₄OH 110:10:1) in dichloromethane) followed by crystallisation with ethyl acetate/heptane 1:1 (v/v) to afford the title compound (49 mg, 48% yield) as an off-white solid. LCMS: m/z 429.1 [M+H]⁺, ESI pos.

### Example 21

### 6-[5-[2-Hydroxy-6-methyl-4-(trifluoromethyl)phenyl]oxazolo[4,5-b]pyridin-2-yl]-1-methyl-5,7-dihydropyrrolo[3,4-b]pyridin-2-one

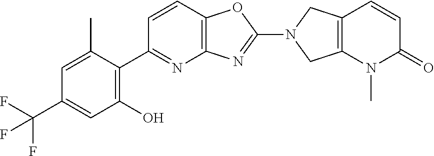 [SMILES: Cc1cc(C(F)(F)F)cc(O)c1-c1ccc2oc(N3Cc4ccc(=O)n(C)c4C3)nc2n1]

### Step A: tert-Butyl 1-methyl-2-oxo-5,7-dihydropyrrolo[3,4-b]pyridine-6-carboxylate

The title compound was obtained as a light brown oil, LCMS: m/z 251.0 [M+H]⁺, ESI pos, using chemistry similar to that described in Example 2, step A starting from tert-butyl 2-oxo-5,7-dihydro-1H-pyrrolo[3,4-b]pyridine-6-carboxylate (Example 20, step B) and iodomethane.

### Step B: 1-Methyl-6,7-dihydro-5H-pyrrolo[3,4-b]pyridin-2-one hydrochloride

The title compound was obtained as a light brown solid, LCMS: m/z 151.0 [M+H]⁺, ESI pos, using chemistry similar to that described in Example 20, step C starting from tert-butyl 1-methyl-2-oxo-5,7-dihydropyrrolo[3,4-b]pyridine-6-carboxylate (Example 21, step A).

### Step C: 6-(5-Chlorooxazolo[4,5-b]pyridin-2-yl)-1-methyl-5,7-dihydropyrrolo[3,4-b]pyridin-2-one

The title compound was obtained as a light grey solid, LCMS: m/z 303.0 [M+H]⁺, ESI pos, using chemistry similar to that described in Example 20, step D starting from 5-chloro-2-(methylthio)oxazolo[4,5-b]pyridine (CAS #1783370-92-2) and 1-methyl-6,7-dihydro-5H-pyrrolo[3,4-b]pyridin-2-one hydrochloride (Example 21, step B).

### Step D: 6-[5-[2-Hydroxy-6-methyl-4-(trifluoromethyl)phenyl]oxazolo[4,5-b]pyridin-2-yl]-1-methyl-5,7-dihydropyrrolo[3,4-b]pyridin-2-one

The title compound was obtained as an off-white foam, LCMS: m/z 443.1 [M+H]⁺, ESI pos, using chemistry similar to that described in Example 20, step E starting from 6-(5-chlorooxazolo[4,5-b]pyridin-2-yl)-1-methyl-5,7-dihydropyrrolo[3,4-b]pyridin-2-one (Example 21, step C) and 3-methyl-2-(4,4,5,5-tetramethyl-1,3,2-dioxaborolan-2-yl)-5-(trifluoromethyl)phenol (CAS #2557358-38-8).

### Example 22

### 6-[5-[2-Hydroxy-6-methyl-4-(trifluoromethyl)phenyl]oxazolo[4,5-b]pyridin-2-yl]-2-methyl-5,7-dihydropyrrolo[3,4-c]pyridazin-3-one

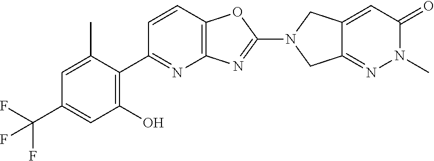 [SMILES: Cc1cc(C(F)(F)F)cc(O)c1-c1ccc2oc(N3Cc4cc(=O)n(C)nc4C3)nc2n1]

### Step A: tert-Butyl 2-methyl-3-oxo-5,7-dihydropyrrolo[3,4-c]pyridazine-6-carboxylate

The title compound was obtained as a dark brown solid, LCMS: m/z 252.1 [M+H]⁺, ESI pos, using chemistry similar to that described in Example 2, step A starting from tert-butyl 3-oxo-5,7-dihydro-2H-pyrrolo[3,4-c]pyridazine-6-carboxylate (CAS #1395493-25-0) and iodomethane.

### Step B: 2-Methyl-6,7-dihydro-5H-pyrrolo[3,4-c]pyridazin-3-one hydrochloride

The title compound was obtained as a black solid, LCMS: m/z 152.0 [M+H]⁺, ESI pos, using chemistry similar to that described in Example 20, step C starting from tert-butyl 2-methyl-3-oxo-5,7-dihydropyrrolo[3,4-c]pyridazine-6-carboxylate (Example 22, step A).

### Step C: 6-(5-Chlorooxazolo[4,5-b]pyridin-2-yl)-2-methyl-5,7-dihydropyrrolo[3,4-c]pyridazin-3-one

To a mixture of 5-chloro-2-(methylthio)oxazolo[4,5-b]pyridine (CAS #1783370-92-2, 130 mg, 0.65 mmol, 1.00 eq) and 2-methyl-6,7-dihydro-5H-pyrrolo[3,4-c]pyridazin-3-one hydrochloride (Example 22, step B) (162 mg, 0.78 mmol, 1.20 eq, 90% purity) in 1,4-dioxane (2.6 mL) and water (0.26 mL) was added N,N-diisopropylethylamine (419 mg, 0.55 mL, 3.24 mmol, 5.00 eq). The reaction mixture was stirred in a sealed tube at 100° C. for 60 hours. The reaction mixture was quenched with water and extracted twice with ethyl acetate. The organic layers were washed with brine. The combined organic layers were dried over sodium sulfate, filtered and concentrated in vacuo. The crude product was adsorbed on ISOLUTE HM-N and purified by flash chromatography (silica gel, gradient 0% to 50% (dichloromethane:methanol:NH₄OH 110:10:1) in dichloromethane) to afford the title compound (32 mg, 15%) as a light brown solid. LCMS: m/z 304.0 [M+H]⁺, ESI pos.

### Step D: 6-[5-[2-Hydroxy-6-methyl-4-(trifluoromethyl)phenyl]oxazolo[4,5-b]pyridin-2-yl]-2-methyl-5,7-dihydropyrrolo[3,4-c]pyridazin-3-one

The title compound was obtained as an off-white solid, LCMS: m/z 444.1 [M+H]⁺, ESI pos, using chemistry similar to that described in Example 20, step E starting from 6-(5-chlorooxazolo[4,5-b]pyridin-2-yl)-2-methyl-5,7-dihydropyrrolo[3,4-c]pyridazin-3-one (Example 22, step C) and 3-methyl-2-(4,4,5,5-tetramethyl-1,3,2-dioxaborolan-2-yl)-5-(trifluoromethyl)phenol (CAS #2557358-38-8).

### Example 23

### 2-[2-(5,7-Dihydropyrrolo[3,4-b]pyrazin-6-yl)oxazolo[4,5-b]pyridin-5-yl]-3-methyl-5-(trifluoromethyl)phenol

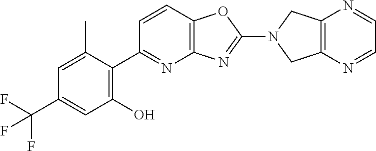 [SMILES: Cc1cc(C(F)(F)F)cc(O)c1-c1ccc2oc(N3Cc4nccnc4C3)nc2n1]

### Step A: 5-Chloro-2-(5,7-dihydropyrrolo[3,4-b]pyrazin-6-yl)oxazolo[4,5-b]pyridine

To a mixture of 5-chloro-2-(methylthio)oxazolo[4,5-b]pyridine (CAS #1783370-92-2, 140 mg, 0.69 mmol, 1.00 eq) and 6,7-dihydro-5H-pyrrolo[3,4-b]pyrazine hydrochloride (CAS #1255099-34-3, 163 mg, 1.04 mmol, 1.50 eq) in 1,4-dioxane (2.0 mL) and water (0.20 mL) was added N,N-diisoproylethylamine (357 mg, 0.47 mL, 2.76 mmol, 4.00 eq). The reaction mixture was stirred at 100° C. for 16 h. The reaction mixture was cooled to room temperature and then diluted with ethyl acetate (2.0 mL) and water (2.0 mL). Let stir for 5 min. Then, the solid was filtered, rinsing with water and diethyl ether to afford the title compound (148 mg, 74% yield) as a grey solid, which was used without further purification. LCMS: m/z 274.0 [M+H]⁺, ESI pos.

### Step B: 2-[2-(5,7-Dihydropyrrolo[3,4-b]pyrazin-6-yl)oxazolo[4,5-b]pyridin-5-yl]-3-methyl-5-(trifluoromethyl)phenol

The title compound was obtained as a white solid, LCMS: m/z 414.2 [M+H]⁺, ESI pos, using chemistry similar to that described in Example 1, step D starting from 5-chloro-2-(5,7-dihydropyrrolo[3,4-b]pyrazin-6-yl)oxazolo[4,5-b]pyridine (Example 23, step A) and 3-methyl-2-(4,4,5,5-tetramethyl-1,3,2-dioxaborolan-2-yl)-5-(trifluoromethyl)phenol (CAS #2557358-38-8).

### Example 24

### 2-[2-(7,8-Dihydro-5H-pyrido[4,3-d]pyrimidin-6-yl)oxazolo[4,5-b]pyridin-5-yl]-3-methyl-5-(trifluoromethyl)phenol

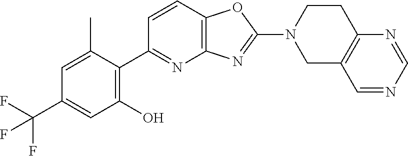 [SMILES: Cc1cc(C(F)(F)F)cc(O)c1-c1ccc2oc(N3CCc4ncncc4C3)nc2n1]

The title compound was obtained as a white solid, LCMS: m/z 428.3 [M+H]⁺, ESI pos, using chemistry similar to that described in Example 1, step D starting from 5-chloro-2-(7,8-dihydro-5H-pyrido[4,3-d]pyrimidin-6-yl)oxazolo[4,5-b]pyridine (Example 17, step A) and 3-methyl-2-(4,4,5,5-tetramethyl-1,3,2-dioxaborolan-2-yl)-5-(trifluoromethyl)phenol (CAS #2557358-38-8).

### Example 25

### 5-Chloro-3-methyl-2-[2-(2,4,6,7-tetrahydropyrazolo[4,3-c]pyridin-5-yl)oxazolo[4,5-b]pyridin-5-yl]phenol

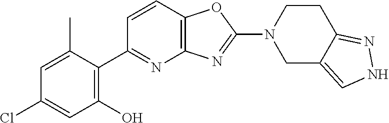 [SMILES: Cc1cc(Cl)cc(O)c1-c1ccc2oc(N3CCc4n[nH]cc4C3)nc2n1]

### Step A: 5-Chloro-2-(2,4,6,7-tetrahydropyrazolo[4,3-c]pyridin-5-yl)oxazolo[4,5-b]pyridine

To a mixture of 5-chloro-2-(methylthio)oxazolo[4,5-b]pyridine (CAS #1783370-92-2, 200 mg, 0.95 mmol, 1.00 eq) and 4,5,6,7-tetrahydro-2H-pyrazolo[4,3-c]pyridine (CAS #933742-87-1, 117 mg, 0.95 mmol, 1.00 eq) in 1,4-dioxane (2.0 mL) was added under argon triethylamine (172 mg, 0.24 mL, 1.70 mmol, 1.80 eq). The reaction mixture was stirred at 95° C. for 16 hours. The reaction mixture was cooled to room temperature and extracted with ethyl acetate and saturated aq. NaHCO₃-solution. The aqueous layer was backextracted three times with ethyl acetate. The organic layers were washed with water and brine. The combined organic layers were dried over sodium sulfate, filtered and concentrated in vacuo. The residue was purified by flash chromatography (silica gel, gradient 0% to 10% methanol in dichloromethane) to afford the title compound (178 mg, 65% yield) as a light yellow solid. LCMS: m/z 275.9 [M+H]⁺, ESI pos.

### Step B: 5-Chloro-3-methyl-2-[2-(2,4,6,7-tetrahydropyrazolo[4,3-c]pyridin-5-yl)oxazolo[4,5-b]pyridin-5-yl]phenol

In a sealed tube, 5-chloro-2-(2,4,6,7-tetrahydropyrazolo[4,3-c]pyridin-5-yl)oxazolo[4,5-b]pyridine (Example 25, step A) (100 mg, 0.33 mmol, 1.00 eq) was dissolved in 1,4-dioxane (2.0 mL) and water (0.50 mL). Then, (4-chloro-2-hydroxy-6-methyl-phenyl)boronic acid (CAS #1207961-50-9, 103 mg, 0.55 mmol, 1.70 eq), cesium carbonate (319 mg, 0.98 mmol, 3.00 eq) and XPhos Pd G3 (41 mg, 0.05 mmol, 0.15 eq) was added under an argon atmosphere. The reaction mixture was stirred at 95° C. for 16 hours. The reaction mixture was quenched with half-saturated aq. NH₄Cl-solution and extracted twice with ethyl acetate. The organic layers were washed with brine. The combined organic layers were dried over sodium sulfate, filtered and concentrated in vacuo. The crude product was adsorbed on ISOLUTE HM-N and purified by flash chromatography (Si-amine, 25 g, gradient 0% to 20% methanol in ethyl acetate) to afford the title compound (75 mg, 57% yield) as a white solid. LCMS: m/z 382.1 [M+H]⁺, ESI pos.

### Example 26

### 5-(Difluoromethyl)-2-[2-(7,8-dihydro-5H-pyrido[4,3-d]pyrimidin-6-yl)oxazolo[4,5-b]pyridin-5-yl]-3-methyl-phenol

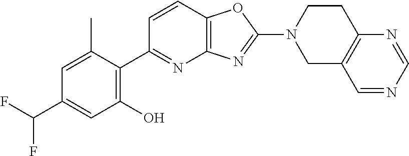 [SMILES: Cc1cc(C(F)F)cc(O)c1-c1ccc2oc(N3CCc4ncncc4C3)nc2n1]

### Step A′: 3-Bromo-5-methyl-benzaldehyde

Two batches were carried out in parallel. To a solution of compound 1 (50.0 g, 200 mmol, 1.0 eq) in THF (500 mL) was added drop wise n-BuLi (2.50 M, 96.0 mL, 1.50 eq) at −70° C. under N₂, then DMF (43.9 g, 600 mmol, 46.2 mL, 2.00 eq) was added and the mixture was stirred at −70° C. for 1 h. TLC (petroleum ether/ethyl acetate=10/1, material R_{f}=0.8, product R_{f}=0.7) showed starting material was consumed and a new spot was formed. The reaction mixture was quenched with water (1000 mL) and extracted with ethyl acetate (3×800 mL). The combined organic layers were dried over anhydrous sodium sulfate, filtered and concentrated under reduced pressure to give a residue. The residue was purified by column chromatography (SiO₂, petroleum ether/ethyl acetate=1/0 to 50/1, product: R_{f}=0.7) to give the title compound (45.0 g, 67% yield) as yellow oil. ¹H NMR: (400 MHz, CDCl₃) δ 9.93 (s, 1H), 7.81 (s, 1H), 7.60 (d, 2H), 2.43 (s, 3H).

### Step B′: 1-Bromo-3-(difluoromethyl)-5-methyl-benzene

To a solution of aforementioned 3-bromo-5-methyl-benzaldehyde (42.0, 211 mmol, 1.0 eq) in DCM (500 mL) was added DAST (74.8 g, 464 mmol, 61.3 mL, 2.2 eq), then the mixture was stirred at 25° C. for 10 hrs. TLC (petroleum ether/ethyl acetate=10/1, material R_{f}=0.500, product R_{f}=0.700) showed starting material was consumed and a new spot was formed. The reaction mixture was quenched with NaHCO₃ (2000 mL) and extracted with DCM (3×1000 mL). The combined organic layers were dried over anhydrous sodium sulfate, filtered and concentrated under reduced pressure to give the title compound (45.0 g, 97% yield) as yellow oil which was used in the next step without further purification.

### Step C′: 2-[3-(Difluoromethyl)-5-methyl-phenyl]-4,4,5,5-tetramethyl-1,3,2-dioxaborolane

To a solution of 1-bromo-3-(difluoromethyl)-5-methyl-benzene (43.0 g, 194 mmol, 1.0 eq), 4,4,5,5-tetramethyl-2-(4,4,5,5-tetramethyl-1,3,2-dioxaborolan-2-yl)-1,3,2-dioxaborolane (59.3 g, 233 mmol, 1.1 eq) and Pd(dppf)Cl₂ (14.2 g, 19.4 mmol, 0.05 eq) in dioxane (400 mL) was added AcOK (40.1 g, 408 mmol, 2.0 eq), then the mixture was stirred at 90° C. for 2 hrs. TLC (petroleum ether/ethyl acetate=5/1, material R_{f}=0.8, product R_{f}=0.7) showed starting material was consumed and a new spot was formed. The reaction mixture was poured into H₂O (200 mL) and extracted with ethyl acetate (3×200 mL). The combined organic layers were dried over anhydrous sodium sulfate, filtered and concentrated under reduced pressure to give the title compound (53.0 g, crude) as black brown oil which was used in the next step without further purification.

### Step D′: 3-(Difluoromethyl)-5-methyl-phenol

To a solution of 2-[3-(difluoromethyl)-5-methyl-phenyl]-4,4,5,5-tetramethyl-1,3,2-dioxaborolane (52.0 g, 194 mmol, 1.0 eq) in THF (800 mL) was added NaOH (2.00 M, 291 mL, 3.0 eq), then H₂O₂ (110 g, 970 mmol, 93.2 mL, 30% purity, 4.0 eq) was added at 0° C. and the mixture was stirred at 25° C. for 3 h. The reaction mixture was then quenched with Na₂SO₃ (1000 mL) and extracted with ethyl acetate (3×500 mL). The combined organic layers were dried over anhydrous sodium sulfate, filtered and concentrated under reduced pressure to give a residue. The crude product was purified by reversed-phase HPLC (0.1% NH₃·H₂O) to yield the title compound (25.0 g, 82% yield) as a brown oil. LCMS: Rₜ=0.463 min, m/z=157.0 [M−H]⁺.

### Step E′: 5-(Difluoromethyl)-2-iodo-3-methyl-phenol

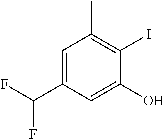

To a solution of 3-(difluoromethyl)-5-methyl-phenol (27.0 g, 171 mmol, 1.0 eq) in toluene (540 mL) was added NaH (13.7 g, 341 mmol, 60% purity, 2.0 eq) at 0° C. under N₂. The mixture was stirred at 0° C. for 0.5 hour then iodine (34.7 g, 136 mmol, 0.8 eq) was added. The mixture was stirred at 25° C. for 1.5 h. The reaction mixture was quenched with aq. NH₄Cl (1000 mL), and extracted with ethyl acetate (3×400 mL). The combined organic layers were dried over Na₂SO₄, filtered and concentrated under reduced pressure. The crude product was purified by reversed-phase HPLC (0.1% FA condition) to give the desired compound and a byproduct (region isomer) as a brown oil. The residue was purified by SFC (column: DAICEL CHIRALPAK AD (250 mm*50 mm, 10 um); mobile phase: [Neu-IPA]; B %: 40%-40%, 2.2 min) to yield the title compound (28.0 g, 73% yield) as a brown oil LCMS: Rₜ=0.499 min, m/z=283.0 [M−H]⁻, ESI neg.

### Step F′: 1-Benzyloxy-5-(difluoromethyl)-2-iodo-3-methyl-benzene

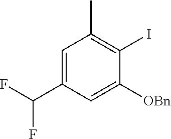 [SMILES: Cc1cc(C(F)F)cc(OCc2ccccc2)c1I]

To a solution of 5-(difluoromethyl)-2-iodo-3-methyl-phenol (19.0 g, 66.9 mmol, 1.0 eq) in DMF (190 mL) was added BnBr (17.2 g, 100 mmol, 11.9 mL, 1.5 eq) and K₂CO₃ (11.1 g, 80.3 mmol, 1.2 eq). The mixture was stirred at 25° C. for 12 hrs. TLC (petroleum ether/ethyl acetate=10/1, material R_{f}=0.4, product R_{f}=0.7) showed the starting material was consumed and a new spot was formed. The reaction mixture was diluted with H₂O (200 mL), and extracted with ethyl acetate (3×200 m). The combined organic layers were washed with H₂O (3×300 mL), dried over Na₂SO₄, filtered and concentrated under reduced pressure to give a residue. The residue was purified by column chromatography (SiO₂, petroleum ether/ethyl acetate=50/1 to 10/1) to give the title compound (22.0 g, 88% yield) as a white solid. ¹H NMR (400 MHz, CDCl₃) δ 7.54 (d, 2H), 7.40-7.44 (m, 2H), 7.33-7.37 (m, 1H), 7.03 (s, 1H), 6.82 (s, 1H), 6.44-6.72 (m, 1H), 5.19 (s, 2H), 2.54 (s, 3H).

### Step G′: 2-[2-Benzyloxy-4-(difluoromethyl)-6-methyl-phenyl]-4,4,5,5-tetramethyl-1,3,2-dioxaborolane

To a solution of 1-benzyloxy-5-(difluoromethyl)-2-iodo-3-methyl-benzene (9.00 g, 24.0 mmol, 1.0 eq) and 2-isopropoxy-4,4,5,5-tetramethyl-1,3,2-dioxaborolane (6.71 g, 36.1 mmol, 7.36 mL, 1.5 eq) in THF (90.0 mL) was added n-BuLi (2.50 M, 14.4 mL, 1.5 eq) at −70° C. under N₂. The mixture was stirred at −60° C. for 1 h. TLC (petroleum ether/ethyl acetate=10/1, material R_{f}=0.70, product R_{f}=0.65) showed the material was consumed completely and a new spot was formed. The reaction mixture was quenched with NH₄Cl aqueous solution (100 mL), and extracted with ethyl acetate (3×100 mL). The combined organic layers were dried over Na₂SO₄, filtered and concentrated under reduced pressure to give a residue. The residue was purified by column chromatography (SiO₂, petroleum ether/ethyl acetate=1/0 to 20/1) to give the target compound (11.2 g, 60% yield, 97% purity) as a white solid. LCMS: m/z=375.3 [M+H]⁺, ESI pos.

### Step H′: 5-(Difluoromethyl)-3-methyl-2-(4,4,5,5-tetramethyl-1,3,2-dioxaborolan-2-yl)phenol

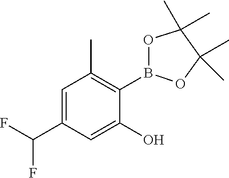 [SMILES: Cc1cc(C(F)F)cc(O)c1B1OC(C)(C)C(C)(C)O1]

To a solution of 2-[2-benzyloxy-4-(difluoromethyl)-6-methyl-phenyl]-4,4,5,5-tetramethyl-1,3,2-dioxaborolane (800 mg, 2.14 mmol, 1.0 eq) and ethyl acetate (20 mL) was added under argon Palladium on carbon (227.5 mg, 213.77 μmol, 0.1 eq). The black suspension was degassed under vacuum and purged with hydrogen several times. The reaction mixture was stirred under hydrogen (balloon) for 4 hours. The reaction mixture was filtered through two glassfibre filters, washed with ethyl acetate (3×20 mL) and evaporated to afford the title compound (595 mg, 89% yield) as colorless oil. LCMS: m/z 285.1 [M+H]⁺, ESI pos.

### Step B: 5-(Difluoromethyl)-2-[2-(7,8-dihydro-5H-pyrido[4,3-d]pyrimidin-6-yl)oxazolo[4,5-b]pyridin-5-yl]-3-methyl-phenol

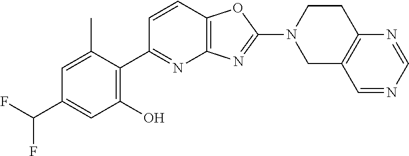 [SMILES: Cc1cc(C(F)F)cc(O)c1-c1ccc2oc(N3CCc4ncncc4C3)nc2n1]

The title compound was obtained as a light yellow solid, LCMS: m/z 410.1 [M+H]⁺, ESI pos, using chemistry similar to that described in Example 1, step D starting from 5-chloro-2-(7,8-dihydro-5H-pyrido[4,3-d]pyrimidin-6-yl)oxazolo[4,5-b]pyridine (Example 17, step A) and 5-(difluoromethyl)-3-methyl-2-(4,4,5,5-tetramethyl-1,3,2-dioxaborolan-2-yl)phenol (Example 26, step H′).

### Example 27

### 7-[5-(4-Chloro-2-hydroxy-6-methyl-phenyl)oxazolo[4,5-b]pyridin-2-yl]-2,5,6,8-tetrahydro-2,7-naphthyridin-3-one

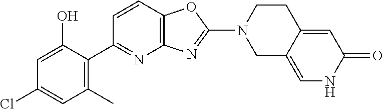 [SMILES: Cc1cc(Cl)cc(O)c1-c1ccc2oc(N3CCc4cc(=O)[nH]cc4C3)nc2n1]

### Step A: tert-Butyl 6-oxo-1,3,4,7-tetrahydro-2,7-naphthyridine-2-carboxylate

The title compound was obtained as a grey solid, LCMS: m/z 251.1 [M+H]⁺, ESI pos, using chemistry similar to that described in Example 20, step B starting from tert-butyl 6-benzyloxy-3,4-dihydro-1H-2,7-naphthyridine-2-carboxylate (Example 19, step A).

### Step B: 5,6,7,8-Tetrahydro-2H-2,7-naphthyridin-3-one hydrochloride

The title compound was obtained as a grey solid, LCMS: m/z 151.1 [M+H]⁺, ESI pos, using chemistry similar to that described in Example 20, step C starting from tert-butyl 6-oxo-1,3,4,7-tetrahydro-2,7-naphthyridine-2-carboxylate (Example 27, step A).

### Step C: 7-(5-Chlorooxazolo[4,5-b]pyridin-2-yl)-2,5,6,8-tetrahydro-2,7-naphthyridin-3-one

The title compound was obtained as a light brown solid, LCMS: m/z 303.0 [M+H]⁺, ESI pos, using chemistry similar to that described in Example 20, step D starting from 5-chloro-2-(methylthio)oxazolo[4,5-b]pyridine (CAS #1783370-92-2) and 5,6,7,8-tetrahydro-2H-2,7-naphthyridin-3-one hydrochloride (Example 27, step B).

### Step D: 7-[5-(4-Chloro-2-hydroxy-6-methyl-phenyl)oxazolo[4,5-b]pyridin-2-yl]-2,5,6,8-tetrahydro-2,7-naphthyridin-3-one

The title compound was obtained as a white amorph freeze-dried solid, LCMS: m/z 409.1 [M+H]⁺, ESI pos, using chemistry similar to that described in Example 25, step B starting from 7-(5-chlorooxazolo[4,5-b]pyridin-2-yl)-2,5,6,8-tetrahydro-2,7-naphthyridin-3-one (Example 27, step C) and (4-chloro-2-hydroxy-6-methyl-phenyl)boronic acid (CAS #1207961-50-9).

### Example 29

### 5-Chloro-2-[2-(1,3-dihydropyrrolo[3,4-c]pyridin-2-yl)oxazolo[4,5-b]pyrazin-5-yl]-3-methyl-phenol; formic acid

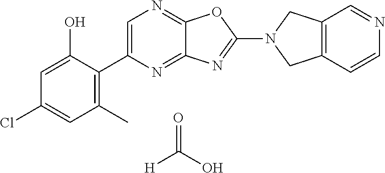 [SMILES: Cc1cc(Cl)cc(O)c1-c1cnc2oc(N3Cc4ccncc4C3)nc2n1 ; O=CO]

### Step A: 3-Benzyloxy-6-bromo-pyrazin-2-amine

To a mixture of BnOH (4.0 g, 36.99 mmol, 1.54 eq) in THF (100 mL) was added NaH (1.4 g, 35.0 mmol, P: 60% in mineral oil, 1.46 eq) in portions at 0° C., then the mixture was heated to 70° C. and stirred for 1 h. The reaction mixture was cooled to 20° C. and 6-bromo-3-chloro-pyrazin-2-amine (CAS #1082843-72-8; 5.0 g, 24 mmol, 1.0 eq) was added. The mixture was stirred for 16 hours at 70° C. Upon the reaction completion, the mixture was quenched with water (150 mL) and extracted with ethyl acetate (150 mL×3), the organic phase was washed with brine (30 mL×2), dried over anhydrous Na₂SO₄, filtered, and concentrated in vacuum. The residue was purified over column chromatography (PE/EtOAc, 5:1) to afford the title compound as a white solid (4.60 g, 67% yield). LCMS: m/z 280.0 [M+H]⁺, ESI pos.

### Step B: N-(3-Benzyloxy-6-bromo-pyrazin-2-yl)-1,3-dihydropyrrolo[3,4-c]pyridine-2-carboxamide; 2,2,2-trifluoroacetic acid

To a solution of 3-benzyloxy-6-bromo-pyrazin-2-amine (1.43 g, 5.11 mmol, 0.8 eq) in THF (20 mL) was added 1,4-nitrophenyl carbonochloridate (CAS #7693-46-1; 1.29 g, 6.39 mmol, 1.0 eq). The mixture was stirred at 60° C. for 1 h. Upon the reaction completion, the mixture was concentrated under vacuum and resolved in DCM (24 mL). Then DIPEA (2.78 mL, 15.9 mmol, 2.5 eq) and 2, 3-dihydro-1H-pyrrolo[3,4-c]pyridine hydrochloride (CAS #6000-50-6; 1.0 g, 6.39 mmol, 1.0 eq) was added in the reaction mixture. The mixture was stirred at 20° C. for 1 hour. Upon the reaction completion, the reaction mixture was added saturated sodium bicarbonate (20 mL) and ethyl acetate (20 mL), and layers were separated. The aqueous phase was extracted with ethyl acetate (10 mL×3). Combined extracts were washed with brine (10 mL×2), dried over Na₂SO₄, filtered, and concentrated under vacuum. The residue was purified by C₁₈ column chromatography (20 g, 0.1% TFA in water/acetonitrile, acetonitrile: 30%-50%) to afford the title compound (900 mg, 33% yield) as a white solid. ¹H NMR (400 MHz, CD₃OD) δ=8.82 (s, 1H), 8.73 (d, 1H), 8.02 (s, 1H), 7.96 (d, 1H), 7.46 (d, 2H), 7.35-7.30 (m, 2H), 7.29-7.23 (m, 1H), 5.44 (s, 2H), 5.09 (s, 2H), 5.05 (s, 2H).

### Step C: N-(6-Bromo-3-hydroxy pyrazin-2-yl)-1,3-dihydro-2H-pyrrolo[3,4-c]pyridine-2-carboxamide

A solution of N-(3-benzyloxy-6-bromo-pyrazin-2-yl)-1,3-dihydropyrrolo[3,4-c]pyridine-2-carboxamide; 2,2,2-trifluoroacetic acid (900 mg, 1.67 mmol, 1.0 eq) in TFA (9.0 mL, 121.2 mmol, 72.7 eq) was stirred at 20° C. for 1 hour. Upon the reaction completion, the reaction mixture was concentrated under vacuum to give a residue, which was dissolved in methanol (5 mL) and basified to pH=9 by addition of NH₃·H₂O resulting in the formation of a white precipitate. After filtration, the residue was air-dried to afford the title compound (670.0 mg, 89% yield) as white solid. ¹H NMR (400 MHz, DMSO-d₆) δ=12.55 (s, 1H), 8.96 (s, 1H), 8.76 (s, 1H), 8.66 (d, 1H), 7.73 (d, 1H), 7.31 (s, 1H), 4.92 (s, 4H).

### Step D: 5-Bromo-2-(1,3-dihydropyrrolo[3,4-c]pyridin-2-yl)oxazolo[4,5-b]pyrazine

To a solution of N-(6-bromo-3-hydroxy-pyrazin-2-yl)-1,3-dihydropyrrolo[3,4-c]pyridine-2-carboxamide (200.0 mg, 0.44 mmol, 1.0 eq) in toluene (2 mL) was added POCl₃ (0.81 mL, 8.89 mmol, 20.0 eq). The reaction mixture was stirred at 100° C. for 2 h. Upon the reaction completion, the reaction mixture was cooled to room temperature. It was slowly pipetted into water (10 mL) and basified to pH=9 by NH₃·H₂O. During this period, white precipitate was formed. It was collected by filtration and air-dried to afford the title compound (110.0 mg, 58% yield) as white solid. ¹H NMR (400 MHz, DMSO-d₆) δ=8.93 (s, 1H), 8.81 (d, 1H), 8.08 (s, 1H), 7.96 (d, 1H), 5.20 (s, 4H).

### Step E: 5-Chloro-2-[2-(1,3-dihydropyrrolo[3,4-c]pyridin-2-yl)oxazolo[4,5-b]pyrazin-5-yl]-3-methyl-phenol; formic acid

To a solution of 5-bromo-2-(1,3-dihydropyrrolo[3,4-c]pyridin-2-yl)oxazolo[4,5-b]pyrazine (50.0 mg, 0.16 mmol, 1.0 eq) in 1,4-dioxane (2 mL) and water (0.4 mL), CsF (95.5 mg, 0.63 mmol, 4.0 eq) and (4-chloro-2-hydroxy-6-methyl-phenyl)boronic acid (23.4 mg, 0.13 mmol, 0.8 eq) was added XPhos Pd G3 (26.6 mg, 0.03 mmol, 0.2 eq). The reaction mixture was stirred at 80° C. for 4 hours under N₂. Upon the reaction completion, the mixture was cooled to room temperature and concentrated under vacuum to give a residue, which was purified by C₁₈ column chromatography (20 g, 0.1% FA in water/acetonitrile, acetonitrile: 30%-40%) and the eluent was lyophilized to give a residue. The residue was triturated in acetonitrile (2 mL) and stirred for 10 mins to afford the title compound (12.0 mg, 18%) as white solid. LCMS: m/z 380.0 [M+H]⁺, ESI pos.

### Example 30

### 5-Chloro-2-[2-(1,3-dihydropyrrolo[3,4-c]pyridin-2-yl)oxazolo[4,5-b]pyrazin-5-yl]-3-(hydroxymethyl)phenol

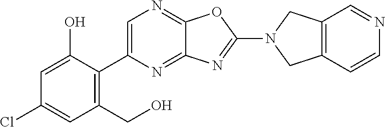 [SMILES: OCc1cc(Cl)cc(O)c1-c1cnc2oc(N3Cc4ccncc4C3)nc2n1]

### Step A′: Methyl 2-amino-5-chloro-3-methoxy-benzoate

To a solution of methyl 2-amino-3-methoxy-benzoate (CAS #5121-34-6, 10.0 g, 55.2 mmol, 1.0 eq) in DMF (100 mL) at 25° C. was added NCS (7.59 g, 56.9 mmol, 1.03 eq). The mixture was then stirred at 50° C. for 2 h. The mixture was poured into water (100 mL) and extracted with EA (3×50 mL), The combined organic layers were washed with brine (50 mL×3), dried over Na₂SO₄, filtered and concentrated under reduced pressure and purified by silica gel column chromatography (petroleum ether:ethyl acetate=1:0 to 5:1) and purified by C₁₈ column chromatography (20 g, 0.1% TFA in water/MeCN), followed by lyophilization to afford the title compound (10.8 g, 91% yield) as a light yellow solid. ¹H NMR (CDCl₃, 400 MHz): δ 7.46 (d, 1H), 6.79 (d, 1H), 6.01 (s, 2H), 3.87 (s, 6H).

### Step B′: Methyl 2-bromo-5-chloro-3-methoxy-benzoate

To a solution of methyl 2-amino-5-chloro-3-methoxy-benzoate (10.0 g, 46.4 mmol, 1.0 eq) in MeCN (80 mL) was added CuBr₂ (20.7 g, 92.8 mmol, 2.0 eq) and CuBr (665.5 mg, 4.64 mmol, 0.1 eq) resulting in a dark color. The mixture was stirred for 20 min at 25° C., and t-BuONO (8.61 g, 83.5 mmol, 1.8 eq) was added dropwise over 10 min. The reaction mixture was stirred for an additional 30 min and then heated at 60° C. for 6 h. The reaction mixture was concentrated in a vacuo then water (150 mL) and EtOAc (100 mL) were added. The resulting mixture was stirred at 25° C. for 30 mins. The organic phase became brown, and the aqueous was green with insoluble materials. The whole mixture was filtered through Celite and washed with EtOAc (100 mL×3). The organic layer was separated, washed with brine (100 mL×3), dried over Na₂SO₄, filtered and concentrated under reduced pressure to give a residue. The residue was purified by column chromatography (SiO₂, petroleum ether/ethyl acetate=10/1 to 3/1) to give the title compound (8.10 g, 62% yield) as a white solid. ¹H NMR (CDCl₃, 400 MHz): δ 7.28 (d, 1H), 6.98 (d, 1H), 3.94 (s, 3H), 3.93 (s, 3H).

### Step C′: 2-Bromo-5-chloro-3-hydroxy-benzoic acid (CAS 1889090-86-1)

To a solution of methyl 2-bromo-5-chloro-3-methoxy-benzoate (8.1 g, 28.98 mmol, 1.0 eq) in DCM (200 mL) was slowly added BBr₃ (8.38 mL, 86.93 mmol, 3.0 eq) at −78° C. under N₂, then the reaction mixture was stirred at 25° C. for 12 h. To the reaction mixture was slowly added MeOH (100 mL), and the resulting mixture was stirred at 20° C. for 30 min. It was mixed with ice-water 500 mL at 0° C., and the organic phase was separated. The aqueous was extracted with DCM (100 mL×3). The combined organic layers were washed with brine (2×200 mL), dried over Na₂SO₄, filtered and concentrated under reduced pressure to give a residue. The residue was purified by column chromatography (SiO₂, petroleum ether/ethyl acetate=10/1 to 0/1) to give the title compound (6.5 g, 89% yield) as a white solid. ¹H NMR (400 MHz, CDCl₃) δ=7.62 (d, 1H), 7.28 (s, 1H), 6.24-6.15 (m, 1H).

### Step D′: 2-Trimethylsilylethoxymethyl-2-bromo-5-chloro-3-(2-trimethylsilylethoxymethoxy)benzoate

A mixture of Cs₂CO₃ (25 g, 77.6 mmol, 3.0 eq) and 2-bromo-5-chloro-3-hydroxy-benzoic acid (6.50 g, 25.9 mmol, 1.0 eq) in DMF (40 mL) was degassed and purged with N₂ three times and stirred for 30 mins. Then SEMCl (12928.7 mg, 77.6 mmol, 3.0 eq) was added to the mixture in one portion. The mixture was stirred at 25° C. for 2 h. The mixture was poured into water (70 mL) and extracted with ethyl acetate (3×40 mL). The combined organic layers were washed with brine (50 mL), dried over anhydrous sodium sulfate, filtered, and the filtrate was concentrated under reduced pressure, Then The residue was purified by silica gel column chromatography (petroleum ether:ethyl acetate=1:0 to 10:1) after concentrated under reduced pressure the title compound (10.8 g, 82% yield) was obtained as colorless oil. ¹H NMR (400 MHz, CD₃OD) δ=7.35 (d, 1H), 7.27 (d, 1H), 5.47 (s, 2H), 5.34 (s, 2H), 3.88-3.76 (m, 4H), 0.99-0.88 (m, 4H), 0.00 (s, 9H), −0.04 (s, 9H).

### Step E′: 2-Bromo-5-chloro-3-(2-trimethylsilylethoxymethoxy)phenyl]methanol

To a solution of 2-trimethylsilylethoxymethyl 2-bromo-5-chloro-3-(2-trimethylsilylethoxy methoxy)benzoate (10.7 g, 20.9 mmol, 1.0 eq) in THF (50 mL) was added 2 M LiBH₄ in THF solution (20.9 mL, 41.8 mmol, 2.0 eq), then the reaction mixture was stirred at 25° C. for 16 h. The mixture was poured into water (100 ml) and extracted with ethyl acetate (50 mL*3), The combined organic layers were washed with brine (100 mL), dried anhydrous sodium sulfate, filtered, and the filtrate was concentrated under reduced pressure The residue was purified by column chromatography (SiO₂, petroleum ether/ethyl acetate=0/1 to 10/1) to afford the title compound (6.0 g, 76% yield) as a colourless oil. ¹H NMR (400 MHz, CD₃OD) δ=7.21 (d, 1H), 7.13 (d, 1H), 5.31 (s, 2H), 4.63 (s, 2H), 3.81 (t, 2H), 0.98 (t, 2H), 0.00 (s, 9H).

### Step F′: 2-[[2-Bromo-3-[[tert-butyl(dimethyl)silyl]oxymethyl]-5-chloro-phenoxy]methoxy]ethyl-trimethyl-silane

A mixture of NaH (163.2 mg, 4.08 mmol, 60%, 1.5 eq) and [2-bromo-5-chloro-3-(2-trimethylsilylethoxymethoxy)phenyl]methanol (1000.0 mg, 2.72 mmol, 1.0 eq) in DMF (10 mL) was degassed and purged with N₂ three times and stirred for 30 min. Then TBSCl (614.79 mg, 4.08 mmol, 1.5 eq) was added to the mixture in one portion. The mixture was stirred at 25° C. for 3 h. The mixture was poured into water (30 mL) and extracted with ethyl acetate (30 mL×3), The combined organic layers were washed with brine (50 mL), dried over anhydrous sodium sulfate, filtered, and the filtrate was concentrated under reduced pressure The residue was purified by column chromatography (SiO₂, petroleum ether/ethyl acetate=0/1 to 20/1) to afford the title compound (600.0 mg, 46% yield) as a colourless oil. ¹H NMR (400 MHz, CD₃OD) δ=7.22 (s, 1H), 7.17 (s, 1H), 5.36 (s, 2H), 4.77 (s, 2H), 3.88 (t, 1H), 1.02-0.98 (s, 9H), 0.97-0.92 (m, 2H), 0.19 (s, 6H), 0.03 (s, 9H).

### Step F: tert-Butyl-[[5-chloro-2-(4,4,5,5-tetramethyl-1,3,2-dioxaborolan-2-yl)-3-(2-trimethylsilylethoxymethoxy)phenyl]methoxy]-dimethyl-silane

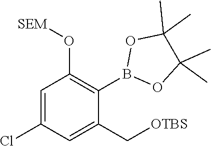 [SMILES: CC1(C)OB(c2c(CO[Si](C)(C)C(C)(C)C)cc(Cl)cc2OCOCC[Si](C)(C)C)OC1(C)C]

To a solution of 2-[[2-bromo-3-[[tert-butyl(dimethyl)silyl]oxymethyl]-5-chloro-phenoxy]methoxy]ethyl-trimethyl-silane (390.0 mg, 0.81 mmol, 1.0 eq) and 4,4,4′,4′,5,5,5′,5′-octamethyl-2,2′-bi(1,3,2-dioxaborolane) (CAS #73183-34-3, 411.04 mg, 1.62 mmol, 2.0 eq) in 1,4-dioxane (4 mL) was added Cs₂CO₃ (527.3 mg, 1.62 mmol, 2.0 eq) and tris(4-methoxy-3,5-dimethylphenyl)phosphane (35.3 mg, 0.08 mmol, 0.1 eq). And the mixture was added Pd(OAc)₂ (18.2 mg, 0.08 mmol, 0.1 eq) under N₂. Finally, the mixture was stirred at 95° C. for 5 hours. The above reaction mixture was cooled to room temperature, then diluted with water (30 mL) and ethyl acetate (30 mL), filtered and the filtrate was extracted with ethyl acetate (30 mL×3). The combined organic phase was washed with brine (40 mL), dried over anhydrous sodium sulfate, filtered and the filtrate was concentrated under reduced pressure. The residue was purified silica gel column chromatography (petroleum ether:ethyl acetate=1:0 to 20:1) to afford the title compound (400.0 mg, 75% yield) as a colourless oil. ¹H NMR (400 MHz, CD₃OD) δ=7.08 (s, 1H), 6.99 (d, 1H), 5.20 (s, 2H), 4.71 (s, 2H), 3.81 (t, 2H), 1.37 (s, 12H), 0.98-0.95 (m, 2H), 0.94 (s, 9H), 0.09 (s, 6H), 0.00 (s, 9H).

### Step A: tert-Butyl-[[5-chloro-2-[2-(1,3-dihydropyrrolo[3,4-c]pyridin-2-yl)oxazolo[4,5-b]pyrazin-5-yl]-3-(2-trimethylsilylethoxymethoxy)phenyl]methoxy]-dimethyl-silane

To a solution of 5-bromo-2-(1,3-dihydropyrrolo[3,4-c]pyridin-2-yl)oxazolo[4,5-b]pyrazine (Example 29, Step D) (46.0 mg, 0.14 mmol, 1.0 eq) and tert-butyl-[[5-chloro-2-(4,4,5,5-tetraethyl-1,3,2-dioxaborolan-2-yl)-3-(2-trimethylsilylethoxymethoxy)phenyl]methoxy]-dimethyl-silane (Step F) (68.9 mg, 0.13 mmol, 0.9 eq), Na₂CO₃ (38.3 mg, 0.36 mmol, 2.5 eq) in 1,4-dioxane (1 mL)/water (0.2 mL) was added XPhos Pd G3 (24.5 mg, 0.03 mmol, 0.2 eq). The reaction mixture was stirred at 80° C. for 4 hours under N₂. Upon the reaction completion, the reaction mixture was cooled to room temperature and concentrated under vacuum to give a residue, which was purified by C₁₈ column chromatography (20 g. 0.1% NH₃·H₂O in water/acetonitrile, acetonitrile: 100%) to afford the title compound (10.0 mg, 10% yield) as colorless oil. LCMS: m/z 640.5 [M+H]⁺, ESI pos.

### Step B: 5-Chloro-2-[2-(1,3-dihydropyrrolo[3,4-c]pyridin-2-yl)oxazolo[4,5-b]pyrazin-5-yl]-3-(hydroxymethyl)phenol

To a solution of tert-butyl-[[5-chloro-2-[2-(1,3-dihydropyrrolo[3,4-c]pyridin-2-yl)oxazolo[4,5-b]pyrazin-5-yl]-3-(2-trimethylsilylethoxymethoxy)phenyl]methoxy]-dimethyl-silane (step A) (10.0 mg, 0.02 mmol, 1.0 eq) in DCM (0.5 mL) was added TFA (0.5 mL, 6.73 mmol, 431 eq). The reaction mixture was stirred at 20° C. for 1.5 hours. Upon the reaction completion, the mixture was concentrated under vacuum. The residue was dissolved in methanol (0.5 mL) and basified to pH=8 by NH₃·H₂O, then purified by preparative HPLC (Method: Column Waters Xbridge 150*25 mm*5 um; Condition: water (NH₄HCO₃)-ACN; Begin B: 20; End B: 50; Gradient Time (min): 100% B; Hold Time (min): 2; Flow Rate (ml/min): 25) to afford the title compound (4.0 mg, 64% yield) as a white solid. LCMS: m/z 396.0 [M+H]⁺, ESI pos.

### Examples 31-42

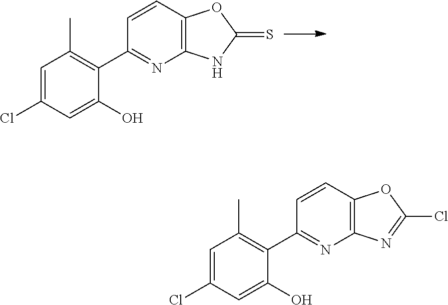 [SMILES: Cc1cc(Cl)cc(O)c1-c1ccc2oc(=S)[nH]c2n1 ; Cc1cc(Cl)cc(O)c1-c1ccc2oc(Cl)nc2n1]

5-(4-Chloro-2-hydroxy-6-methyl-phenyl)-3H-oxazolo[4,5-b]pyridine-2-thione Intermediate 1 (1320.0 mg, 4.51 mmol, 1.0 eq) was dissolved in DCM (50 mL) and oxalyl chloride (5.8 mL, 67.64 mmol, 15.0 eq) was added, followed by drop-wise addition of DMF (1.5 mL). The mixture was stirred at room temperature for 30 min, then concentrated in vacuo. The resulting residue was taken up in DCM (45 mL) and sat. aq. K₂CO₃ (15 mL) was added. The DCM layer was isolated and the aqueous back-extracted with DCM (30 mL). The combined organic extracts were dried using a phase separator. The resulting solution was made up to 92 mL with DCM and used as a stock solution in a series of reactions using the following general procedure:

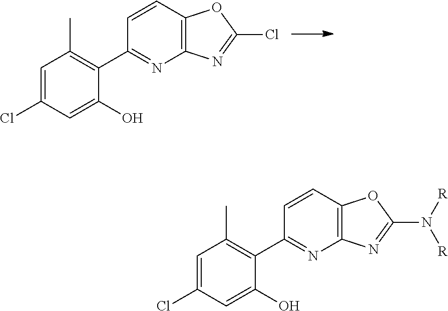

Amine (5.41 mmol, 1.2 eq) was dissolved in DMF (0.1 mL) and triethylamine (0.02 mL, 0.15 mmol, 3.0 eq) was added, followed by 1 mL of the stock solution of 5-chloro-2-(2-chlorooxazolo[4,5-b]pyridin-5-yl)-3-methylphenol (1 eq). The mixture was shaken at room temperature for 20 min. The DCM was evaporated overnight, and the resulting residue made up to 1 mL with DMF, then filtered. The resulting filtrate was purified by prep HPLC.

The following examples ‘Ex.’ were synthesised by method outlined above:

| Ex- | Structure | | |
|---|---|---|---|
| am- | (All examples containing | | Amine name, Prep |
| ple | chiral centres are | | Gradient, |
| No | racemates unless stated) | Name | LCMS data |
| 31 | 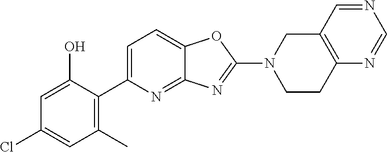 [SMILES: Cc1cc(Cl)cc(O)c1-c1ccc2oc(N3CCc4ncncc4C3)nc2n1] | 5-Chloro-2-[2-(7,8- dihydro-5H- pyrido[4,3- d]pyrimidin-6- yl)oxazolo[4,5- b]pyridin-5-yl]-3- methyl-phenol | 5,6,7,8- Tetrahydropyrido[4, 3-d]pyrimidine (CAS # 192869-50-4), 20-50% MeCN in water, LCMS m/z 393.9 [M + H]⁺, ESI pos. |
| 32 | 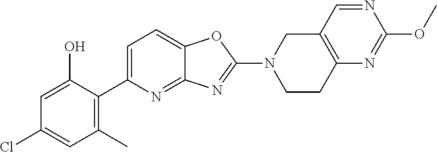 [SMILES: COc1ncc2c(n1)CCN(c1nc3nc(-c4c(C)cc(Cl)cc4O)ccc3o1)C2] | 5-Chloro-2-[2-(2- methoxy-7,8-dihydro- 5H-pyrido[4,3- d]pyrimidin-6- yl)oxazolo[4,5- b]pyridin-5-yl]-3- methyl-phenol | 2-Methoxy-5,6,7,8- tetrahydropyrido[4,3- d]pyrimidine (CAS # 880361-83-1), 30-60% MeCN in water, LCMS m/z 423.9 [M + H]⁺, ESI pos. |
| 33 | 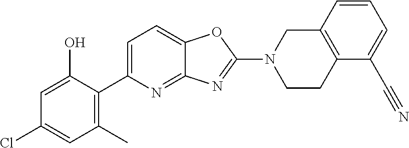 [SMILES: Cc1cc(Cl)cc(O)c1-c1ccc2oc(N3CCc4c(C#N)cccc4C3)nc2n1] | 2-(5-(4-Chloro-2- hydroxy-6- methylphenyl)oxazolo [4,5-b]pyridin-2-yl)- 1,2,3,4- tetrahydroisoquinoline- 5-carbonitrile | 1,2,3,4- tetrahydroisoquinoline- 5-carbonitrile (CAS # 215794-24- 4), 40-70% MeCN in water, LCMS m/z 417.0 [M + H]⁺, ESI pos. |
| 34 | 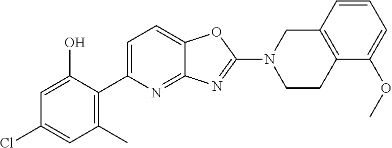 [SMILES: COc1cccc2c1CCN(c1nc3nc(-c4c(C)cc(Cl)cc4O)ccc3o1)C2] | 5-chloro-2-[2-(5- methoxy-3,4-dihydro- 1H-isoquinolin-2- yl)oxazolo[4,5- b]pyridin-5-yl]-3- methyl-phenol | 5-Methoxy-1,2,3,4- tetrahydroisoquinoline hydrochloride (CAS # 103030-69- 9), 45-75% MeCN in water, LCMS m/z 422.0 [M + H]⁺, ESI pos. |
| 35 | 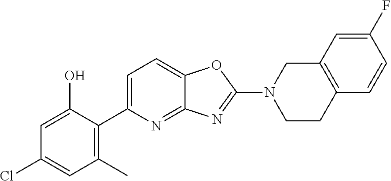 [SMILES: Cc1cc(Cl)cc(O)c1-c1ccc2oc(N3CCc4ccc(F)cc4C3)nc2n1] | 5-Chloro-2-[2-(7- fluoro-3,4-dihydro- 1H-isoquinolin-2- yl)oxazolo[4,5- b]pyridin-5-yl]-3- methyl-phenol | 7-Fluoro-1,2,3,4- tetrahydroisoquinoline hydrochloride (CAS # 406923-91- 9), 40-70% MeCN in water, LCMS m/z 410.0 [M + H]⁺, ESI pos. |
| 36 | 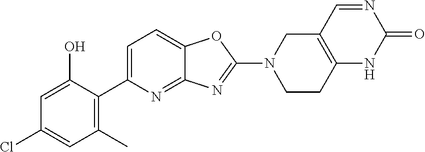 [SMILES: Cc1cc(Cl)cc(O)c1-c1ccc2oc(N3CCc4[nH]c(=O)ncc4C3)nc2n1] | 6-[5-(4-Chloro-2- hydroxy-6-methyl- phenyl)oxazolo[4,5- b]pyridin-2-yl]- 1,5,7,8- tetrahydropyrido[4,3- d]pyrimidin-2-one | 5,6,7,8- Tetrahydropyrido[4, 3-d]pyrimidin-2-ol hydrochloride (CAS # 1956321-99-5), 5-20% MeCN in water, LCMS m/z 410.1 [M + H]⁺, ESI pos. |
| 37 | 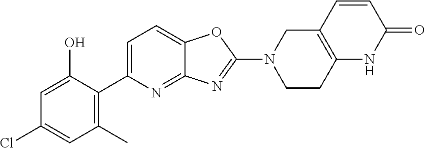 [SMILES: Cc1cc(Cl)cc(O)c1-c1ccc2oc(N3CCc4[nH]c(=O)ccc4C3)nc2n1] | 6-[5-(4-Chloro-2- hydroxy-6-methyl- phenyl)oxazolo[4,5- b]pyridin-2-yl]- 1,5,7,8-tetrahydro-1,6- naphthyridin-2-one | 5,6,7,8-Tetrahydro- 1,6-naphthyridin- 2(1H)-one (CAS # 676994-64-2), 5-35% MeCN in water, LCMS m/z 409.1 [M + H]⁺, ESI pos. |
| 38 | 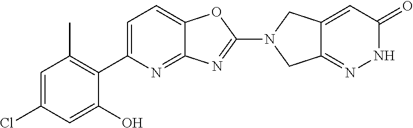 [SMILES: Cc1cc(Cl)cc(O)c1-c1ccc2oc(N3Cc4cc(=O)[nH]nc4C3)nc2n1] | 6-[5-(4-chloro-2- hydroxy-6-methyl- phenyl)oxazolo[4,5- b]pyridin-2-yl]-5,7- dihydro-2H- pyrrolo[3,4- c]pyridazin-3-one | 2,5,6,7-Tetrahydro- 3H-pyrrolo[3,4- c]pyridazin-3-one hydrochloride (CAS # 1415550-33-2), 5-35% MeCN in water, LCMS m/z 396.2 [M + H]⁺, ESI pos. |
| 40 | 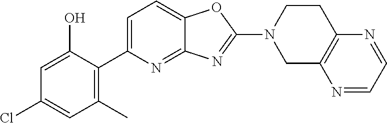 [SMILES: Cc1cc(Cl)cc(O)c1-c1ccc2oc(N3CCc4nccnc4C3)nc2n1] | 5-Chloro-2-[2-(7,8- dihydro-5H- pyrido[3,4-b]pyrazin- 6-yl)oxazolo[4,5- b]pyridin-5-yl]-3- methyl-phenol | 5H,6H,7H,8H- pyrido[3,4- b]pyrazine (CAS # 405162-62-1), 25-55% MeCN in water, LCMS m/z 394.0 [M + H]⁺, ESI pos. |
| 42 | 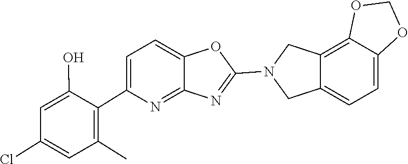 [SMILES: Cc1cc(Cl)cc(O)c1-c1ccc2oc(N3Cc4ccc5c(c4C3)OCO5)nc2n1] | 5-Chloro-2-(2-(6,8- dihydro-7H- [1,3]dioxolo[4,5- e]isoindol-7- yl)oxazolo[4,5- b]pyridin-5-yl)-3- methylphenol | 7,8-Dihydro-6H- [1,3]dioxolo[4,5- e]isoindole hydrochloride (CAS # 1998216-16-2), 40-70% MeCN in water, LCMS m/z 421.9 [M + H]⁺, ESI pos. |

### Example 41

### 6-[5-[2-Hydroxy-6-methyl-4-(trifluoromethyl)phenyl]oxazolo[4,5-b]pyridin-2-yl]-3,5,7,8-tetrahydropyrido[4,3-d]pyrimidin-4-one

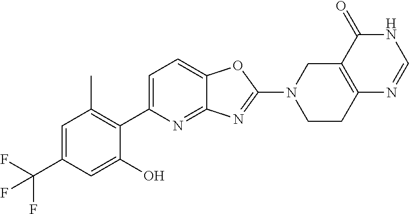 [SMILES: Cc1cc(C(F)(F)F)cc(O)c1-c1ccc2oc(N3CCc4nc[nH]c(=O)c4C3)nc2n1]

5-[2-Hydroxy-6-methyl-4-(trifluoromethyl)phenyl]-3H-oxazolo[4,5-b]pyridine-2-thione Intermediate 2 (156.0 mg, 0.33 mmol, 1.0 eq) was dissolved in DCM (2 mL) and oxalyl chloride (0.59 mL, 6.87 mmol, 21.1 eq) was added. The mixture was stirred at room temperature for 30 min, then concentrated in vacuo. The resulting residue was taken up in DCM (10 mL) and diluted with sat. aq. K₂CO₃ (10 mL) after which the layers were separated. The aqueous layer was extracted with DCM (2×10 mL) after which the combined organics were concentrated in vacuo. The resulting solution was dissolved in DCM (4 mL) to make a stock solution. Then 2 mL of this stock solution was added to 5,6,7,8-tetrahydro-3H-pyrido[4,3-d]pyrimidin-4-one; dihydrochloride (59.0 mg, 0.26 mmol, 1.6 eq), triethylamine (0.21 mL, 1.5 mmol, 9 eq) and DMF (0.1 mL). The reaction mixture was stirred at r.t. for 16 hr after which it was concentrated in vacuo. The resulting product was dissolved in DMF (1 mL), filtered and the resulting filtrate was purified by prep HPLC (25-100% MeCN in water) to afford the title compound as a white solid. LCMS m/z 444.2 [M+H]⁺, ESI pos.

### Example 43

### 2-[5-[2-Hydroxy-6-methyl-4-(trifluoromethyl)phenyl]oxazolo[4,5-b]pyrazin-2-yl]-1,3-dihydropyrrolo[3,4-c]pyridin-6-ol; 2,2,2-trifluoroacetic acid

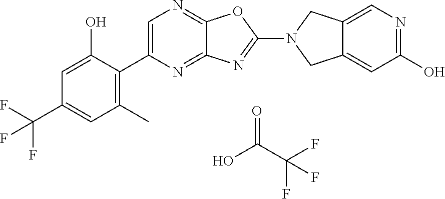 [SMILES: Cc1cc(C(F)(F)F)cc(O)c1-c1cnc2oc(N3Cc4cnc(O)cc4C3)nc2n1 ; O=C(O)C(F)(F)F]

### Step A: N-(3-benzyloxy-6-chloro-pyrazin-2-yl)-6-methoxy-1,3-dihydropyrrolo[3,4-c]pyridine-2-carboxamide

To a solution of 3-benzyloxy-6-chloro-pyrazin-2-amine (1.37 g, 5.79 mmol, 1.0 eq; CAS #2923540-12-7) in THF (13 mL) was added 4-nitrophenyl carbonochloridate (1.28 g, 6.37 mmol, 1.1 eq; CAS #7693-46-1). The mixture was stirred at 60° C. for 1 hour. Upon reaction completion, the reaction mixture was concentrated under reduced pressure. The residue was dissolved in DCM (8 mL), then DIPEA (1.87 g, 14.48 mmol, 2.5 eq) and 6-methoxy-2,3-dihydro-1H-pyrrolo[3,4-c]pyridine (870.0 mg, 5.79 mmol, 1.0 eq) (Example 12, step A) was added to the above reaction mixture and stirring was continued at 20° C. for 1 hour. The mixture was poured into water (20 mL) and extracted with EtOAc (30 mL*3). The combined organic layers were washed with brine (20 mL), dried over anhydrous sodium sulfate, filtered and the filtrate was concentrated under reduced pressure. The residue was purified by column chromatography on silica gel (petroleum ether/ethyl acetate=0/1 to 1/1) to afford the title compound (1300.0 mg, 49% yield) as a white solid. ¹H NMR (400 MHz, DMSO-d₆): δ 9.32 (s, 1H), 8.16 (s, 1H), 8.02 (s, 1H), 7.52-7.42 (m, 3H), 7.35-7.25 (m, 2H), 6.83 (s, 1H), 5.39 (s, 2H), 4.90-4.60 (m, 4H), 3.85 (s, 3H).

### Step B: N-[3-benzyloxy-6-[2-hydroxy-6-methyl-4-(trifluoromethyl)phenyl]pyrazin-2-yl]-6-methoxy-1,3-dihydropyrrolo[3,4-c]pyridine-2-carboxamide

To a mixture of CsF (1.03 g, 6.8 mmol, 4.0 eq), 3-methyl-2-(4,4,5,5-tetramethyl-1,3,2-dioxaborolan-2-yl)-5-(trifluoromethyl)phenol (747.6 mg, 3.4 mmol, 2.0 eq; CAS #2557358-38-8) and aforementioned N-(3-benzyloxy-6-chloro-pyrazin-2-yl)-6-methoxy-1,3-dihydropyrrolo[3,4-c]pyridine-2-carboxamide (700.0 mg, 1.7 mmol, 1.0 eq.) in 1,4-dioxane (10 mL) and water (1 mL) was added XPhos Pd G₃ (285.8 mg, 0.34 mmol, 0.2 eq). The mixture was stirred at 95° C. for 5 hours under N₂. Upon reaction completion, it was cooled to room temperature and filtered. Then, the filtrate was concentrated under reduced pressure and the residue was purified by column chromatography on silica gel (petroleum ether/ethyl acetate=0/1 to 0/1) to give the title compound (360.0 mg, 38% yield) as a light yellow solid. LCMS: m/z 552.1 [M+H]⁺, ESI⁺ pos.

### Step C: N-[3-Benzyloxy-6-[2-methoxy-6-methyl-4-(trifluoromethyl)phenyl]pyrazin-2-yl]-6-methoxy-1,3-dihydropyrrolo[3,4-c]pyridine-2-carboxamide

To a mixture of aforementioned N-[3-benzyloxy-6-[2-hydroxy-6-methyl-4-(trifluoromethyl)phenyl]pyrazin-2-yl]-6-methoxy-1,3-dihydropyrrolo[3,4-c]pyridine-2-carboxamide (210.0 mg, 0.38 mmol, 1.0 eq) and K₂CO₃ (68.3 mg, 0.49 mmol, 1.3 eq) in DMF (3 mL) was added Mel (63.2 mg, 0.38 mmol, 1.0 eq). The mixture was stirred at 25° C. for 12 hours under nitrogen. The mixture was then poured into water (10 mL) and extracted with EtOAc (10 mL*3). The combined organic layers were washed with brine (20 mL), dried over anhydrous sodium sulfate, filtered, and the filtrate was concentrated under reduced pressure. The residue was purified by prep-TLC (SiO₂, petroleum ether:ethyl acetate=2:1) to afford the title compound (137.0 mg, 63% yield) as a light yellow solid. LCMS: m/z 566.1 [M+H]⁺, ESI⁺ pos.

### Step D: N-[3-Hydroxy-6-[2-methoxy-6-methyl-4-(trifluoromethyl)phenyl]pyrazin-2-yl]-6-methoxy-1,3-dihydropyrrolo[3,4-c]pyridine-2-carboxamide

A solution of aforementioned N-[3-benzyloxy-6-[2-methoxy-6-methyl-4-(trifluoromethyl)phenyl]pyrazin-2-yl]-6-methoxy-1,3-dihydropyrrolo[3,4-c]pyridine-2-carboxamide (137.0 mg, 0.24 mmol, 1.0 eq) in TFA (3.0 mL) was stirred at 25° C. for 3 hours. The mixture was concentrated under reduced pressure and the residue was purified by prep-HPLC (column: Phenomenex Gemini, 150 mm*30 mm*15 um; mobile phase: [water (0.1% TFA, V/V)-MeCN]; B %: 22%-52%, 12 mins) to afford the title compound (100.0 mg, 88% yield) as a yellow solid. LCMS: m/z 476.1 [M+H]⁺, ESI⁺ pos.

### Step E: 2-(6-Methoxy-1,3-dihydropyrrolo[3,4-c]pyridin-2-yl)-5-[2-methoxy-6-methyl-4-(trifluoromethyl)phenyl]oxazolo[4,5-b]pyrazine

A solution of aforementioned N-[3-hydroxy-6-[2-methoxy-6-methyl-4-(trifluoromethyl)phenyl]pyrazin-2-yl]-6-methoxy-1,3-dihydropyrrolo[3,4-c]pyridine-2-carboxamide (90.0 mg, 0.19 mmol, 1.0 eq) in POCl₃ (5.0 mL) was stirred at 90° C. for 3 hours. Upon reaction completion, the mixture was cooled to room temperature and concentrated under reduced pressure. The residue was then purified by prep-HPLC (column: Phenomenex Gemini, 150 mm*30 mm*15 um; mobile phase: [water (0.1% TFA, V/V)-MeCN]; B %: 22%-52%, 12 mins), to afford the title compound (41.0 mg, 43% yield) as a light yellow solid. ¹H NMR (400 MHz, DMSO-d₆): δ8.27 (s, 1H), 7.84 (s, 1H), 7.39 (s, 1H), 7.29 (s, 1H), 6.98 (s, 1H), 5.12-5.00 (m, 4H), 3.89 (s, 3H), 3.76 (s, 3H), 2.15 (s, 3H).

### Step F: 2-[2-(6-Methoxy-1,3-dihydropyrrolo[3,4-c]pyridin-2-yl)oxazolo[4,5-b]pyrazin-5-yl]-3-methyl-5-(trifluoromethyl)phenol

To a solution of 2-(6-methoxy-1,3-dihydropyrrolo[3,4-c]pyridin-2-yl)-5-[2-methoxy-6-methyl-4-(trifluoromethyl)phenyl]oxazolo[4,5-b]pyrazine (14.0 mg, 0.03 mmol, 1.0 eq) in DCM (0.5 mL) was added BBr₃ (0.06 mL, 0.61 mmol, 20.0 eq) at 0° C. The mixture was stirred at 25° C. for 0.5 hour. Afterwards, the reaction mixture was quenched by addition of MeOH (1 mL) and subsequently concentrated under reduced pressure. The residue was purified by prep-HPLC (column: Phenomenex Gemini, 150 mm*30 mm*15 um; mobile phase: [water (0.1% TFA, V/V)-MeCN]; B %: 30%-52%, 10 mins) to afford the title compound (12.0 mg, 88% yield) as a yellow solid. ¹H NMR (400 MHz, CD₃OD): δ 8.25 (s, 1H), 7.81 (s, 1H), 7.11 (s, 1H), 7.01 (s, 1H), 6.96 (s, 1H), 5.11-5.00 (m, 4H), 3.97 (s, 3H), 2.22 (s, 3H).

### Step G: 2-[5-[2-Hydroxy-6-methyl-4-(trifluoromethyl)phenyl]oxazolo[4,5-b]pyrazin-2-yl]-1,3-dihydropyrrolo[3,4-c]pyridin-6-ol; 2,2,2-trifluoroacetic acid

A mixture of 2-[2-(6-methoxy-1,3-dihydropyrrolo[3,4-c]pyridin-2-yl)oxazolo[4,5-b]pyrazin-5-yl]-3-methyl-5-(trifluoromethyl)phenol (10.0 mg, 0.02 mmol, 1.0 eq) in HBr/AcOH (0.5 mL) was stirred at 80° C. for 0.5 hour. Upon reaction completion, the mixture was cooled to 20° C. and then concentrated under reduced pressure. The residue was purified by prep-HPLC (column: Phenomenex Gemini, 150 mm*30 mm*15 um; mobile phase: [water (0.1% TFA, V/V)-MeCN]; B %: 30%-52%, 10 mins) to afford the title compound (1.56 mg, 16% yield) as a white solid. LCMS: m/z 430.2 [M+H]⁺, ESI⁺ pos.

### Example 44

### 6-[5-[2-Hydroxy-6-methyl-4-(trifluoromethyl)phenyl]oxazolo[4,5-b]pyridin-2-yl]-5,7-dihydro-1H-pyrrolo[3,4-d]pyrimidine-2,4-dione

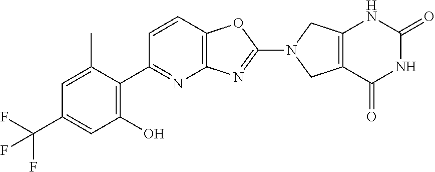 [SMILES: Cc1cc(C(F)(F)F)cc(O)c1-c1ccc2oc(N3Cc4[nH]c(=O)[nH]c(=O)c4C3)nc2n1]

### Step A: 6-(5-Chlorooxazolo[4,5-b]pyridin-2-yl)-5,7-dihydro-1H-pyrrolo[3,4-d]pyrimidine-2,4-dione

To a solution of 5-chloro-2-(methylthio)oxazolo[4,5-b]pyridine (CAS #1783370-92-2, 200 mg, 0.95 mmol, 1.0 eq) in 1,4-dioxane (2 mL) was added 1,5,6,7-tetrahydropyrrolo[3,4-d]pyrimidine-2,4-quinone; hydrochloride (CAS #13931-24-3, 199 mg, 1.05 mmol, 1.1 eq) followed by triethylamine (CAS #121-44-8, 211 mg, 0.29 mL, 2.08 mmol, 2.2 eq). The reaction mixture was stirred at 90° C. for 16 hours. Afterwards, the solvent was evaporated. The residue was dissolved in N-methyl-2-pyrrolidinone (CAS #872-50-4, 12 mL) and triethylamine (210 mg, 0.29 mL, 2.08 mmol, 2.2 eq) was added to the reaction mixture, and stirring was continued at 130° C. for 16 hours. Then, the reaction mixture was cooled to room temperature, adsorbed on ISOLUTE HM-N and purified by flash chromatography (ISCO, 12 g SiO₂: 0% to 10% MeOH in DCM) to afford the title compound (340 mg, 99% yield) as brown solid. LCMS: m/z 306.0 [M+H]⁺, ESI pos.

### Step B: 6-[5-[2-Hydroxy-6-methyl-4-(trifluoromethyl)phenyl]oxazolo[4,5-b]pyridin-2-yl]-5,7-dihydro-1H-pyrrolo[3,4-d]pyrimidine-2,4-dione

A mixture of 6-(5-chlorooxazolo[4,5-b]pyridin-2-yl)-5,7-dihydro-1H-pyrrolo[3,4-d]pyrimidine-2,4-quinone (Example 44, step A) (50 mg, 0.14 mmol, 1.0 eq), 3-methyl-2-(4,4,5,5-tetramethyl-1,3,2-dioxaborolan-2-yl)-5-(trifluoromethyl)phenol (72 mg, 0.215 mmol, 1.44 eq), cesium carbonate (137 mg, 0.42 mmol, 3.0 eq) and XPhos Pd G3 (12.4 mg, 0.015 mmol, 0.105 eq) in 1,4-dioxane (0.23 mL) and water (58 μL) was flushed with argon and stirred at 90° C. for two hours. The reaction mixture was cooled to room temperature and extracted with dichloromethane/methanol (19:1) and half-saturated aq. NH₄Cl-solution. The organic layer was washed with water and brine. The aqueous layers were back-extracted twice with a mixture of dichloromethane/methanol (19:1). The combined organic layers were dried over sodium sulfate, filtered and concentrated in vacuo. The crude was then purified by preparative HPLC (Column: YMC-Trail, 12 nm, 5 μm, 100×30 mm; Condition: ACN/water+0.1% HCOOH; Gradient: ACN in water, runtime 4.5 min) to afford the title compound (8 mg, 12% yield) as light grey solid. LCMS: m/z 446.1 [M+H]⁺, ESI pos

### Example A

A compound of formula I can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. A compound of formula I: 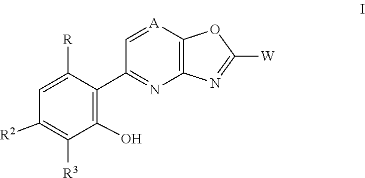
wherein,
R¹ is H, alkyl, hydroxyalkyl or alkoxyalkyl;
R² is halo, haloalkyl or cyano;
R³ is H;
or R² and R³, and the atoms to which they are bonded, form a 4-6 membered heterocycle ring comprising a single O heteroatom optionally substituted with one or two substituents independently selected from halo and alkyl,
or R² and R³, and the atoms to which they are bonded, form a 3-6 membered cycloalkyl ring optionally substituted with one or two substituents independently selected from halo and alkyl;
A is CH or N;
W is: 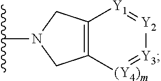
p is 1 or 2;
q is 1 or 2;
m is 0 or 1;
Y₁ is C═O, CH, CH₂, C—R^{x}, N, or NH;
Y₂ is C═O, CH, C—R^{y}, O, N, NH, or N—CH₃;
Y₃ is C═O, CH, CH₂, CR^{y}, N, NH, or NR^{z};
Y₄ is C═O, CH, CR^{y}, N, NH or N—CH₃;
wherein R^{x} is H and each R^{y} is independently selected from —OH, alkyl, alkoxy, cyano and halo, or R^{x}+R^{y}, through the atoms to which they are attached, form a heterocycle ring;
R^{z} is H, alkyl or hydroxyalkyl;
and pharmaceutically acceptable salts thereof.

2. A compound according to claim 1, wherein R¹ is alkyl or hydroxyalkyl.

3. A compound according to claim 1, wherein R¹ is alkyl.

4. A compound according to claim 1, wherein n is 1, q is 1, and m is 1.

5-6. (canceled)

7. A compound according to claim 1, wherein Y₁ is CH, C—R^{x}, N, or NH.

8. A compound according to claim 1, wherein Y₁ is CH, N, or NH.

9. (canceled)

10. A compound according to claim 1, wherein Y₂ is C═O, CH, C—R^{y}, N, or NH.

11. (canceled)

12. A compound according to claim 1, wherein Y₃ is C═O, CH, C—R^{y}, N, NH, or NR_{z}.

13-14. (canceled)

15. A compound according to claim 1, wherein each R^{y} is independently selected from OH, alkyl, alkoxy, cyano, and halo.

16. (canceled)

17. A compound according to claim 1, wherein each R^{z} is H, alkyl, or hydroxyalkyl.

18. (canceled)

19. A compound according to claim 1, wherein,
R¹ is alkyl or hydroxyalkyl;
R² is halo, haloalkyl, or cyano;
R³ is H;
A is CH or N;
p is 1 or 2;
q is 1 or 2;
m is 0 or 1;
Y₁ is CH, C—R^{x}, N, or NH;
Y₂ is C═O, CH, C—R^{y}, N, or NH;
Y₃ is C═O, CH, CR^{y}, N, NH, or NR^{z};
Y₄ is C═O, CH, CR^{y}, N, NH, or N—CH₃;
wherein R^{x} is H and each R^{y} is independently selected from OH, alkyl, alkoxy, cyano and halo, or R^{x}+R^{y}, through the atoms to which they are attached, form a heterocycle ring comprising 1 or 2 oxygen heteroatoms;
R^{z} is H, alkyl, or hydroxyalkyl;
and pharmaceutically acceptable salts thereof.

20. A compound according to claim 1, wherein,
R¹ is alkyl;
R² is halo, haloalkyl, or cyano;
R³ is H;
A is CH or N;
p is 1 or 2;
q is 1;
m is 0 or 1;
Y₁ is CH, N, or NH;
Y₂ is C═O, CH, C—R^{y}, N, or NH;
Y₃ is CH, N, NH, or NR^{z};
Y₄ is CH or N;
wherein each R^{y} is alkyl;
R^{z} is H, alkyl, or hydroxyalkyl;
and pharmaceutically acceptable salts thereof.

21. A compound according to claim 1, wherein,
R¹ is alkyl;
R² is halo, haloalkyl, or cyano;
R³ is H;
A is CH or N;
p is 1 or 2;
q is 1;
m is 0 or 1;
Y₁ is CH, N, or NH;
Y₂ is C═O, CH, C—R^{y}, N, or NH;
Y₃ is CH, N, NH or NR^{z}; and
Y₄ is CH or N;
wherein each R^{y} is alkyl; and R^{z} is hydroxyalkyl,
and pharmaceutically acceptable salts thereof.

22. A compound according to claim 1, wherein,
R¹ is alkyl;
R² is halo, haloalkyl, or cyano;
R³ is H;
A is CH or N;
p is 1;
q is 1;
m is 1;
Y₁ is CH;
Y₂ is C═O or N;
Y₃ is CH, NH or NR^{z};
Y₄ is CH;
R^{z} is hydroxyalkyl;
and pharmaceutically acceptable salts thereof.

23. A compound according to claim 1, selected from:
2-[5-[2-Hydroxy-6-methyl-4-(trifluoromethyl)phenyl]oxazolo[4,5-b]pyridin-2-yl]-3,5-dihydro-1H-pyrrolo[3,4-c]pyridin-6-one;
2-[5-[2-Hydroxy-6-methyl-4-(trifluoromethyl)phenyl]oxazolo[4,5-b]pyridin-2-yl]-5-methyl-1,3-dihydropyrrolo[3,4-c]pyridin-6-one;
3-Hydroxy-5-methyl-4-[2-(6-oxo-3,5-dihydro-1H-pyrrolo[3,4-c]pyridin-2-yl)oxazolo[4,5-b]pyridin-5-yl]benzonitrile;
2-[5-(4-Chloro-2-hydroxy-6-methyl-phenyl)oxazolo[4,5-b]pyridin-2-yl]-3,5-dihydro-1H-pyrrolo[3,4-c]pyridin-6-one;
5-(2-Hydroxyethyl)-2-[5-[2-hydroxy-6-methyl-4-(trifluoromethyl)phenyl]oxazolo[4,5-b]pyridin-2-yl]-1,3-dihydropyrrolo[3,4-c]pyridin-6-one;
5-Chloro-2-(2-isoindolin-2-yloxazolo[4,5-b]pyridin-5-yl)-3-methyl-phenol;
5-chloro-2-[2-(1,3-dihydropyrrolo[3,4-c]pyridin-2-yl)oxazolo[4,5-b]pyridin-5-yl]-3-methyl-phenol;
2-[5-(4-Chloro-2-hydroxy-6-methyl-phenyl)oxazolo[4,5-b]pyridin-2-yl]isoindolin-5-ol;
5-Chloro-2-[2-(4-fluoroisoindolin-2-yl)oxazolo[4,5-b]pyridin-5-yl]-3-methyl-phenol;
4-[2-(1,3-dihydropyrrolo[3,4-c]pyridin-2-yl)oxazolo[4,5-b]pyridin-5-yl]-3-hydroxy-5-methyl-benzonitrile;
5-chloro-3-methyl-2-[2-(2-methyl-7,8-dihydro-5H-pyrido[4,3-d]pyrimidin-6-yl)oxazolo[4,5-b]pyridin-5-yl]phenol;
2-[2-(6-methoxy-1,3-dihydropyrrolo[3,4-c]pyridin-2-yl)oxazolo[4,5-b]pyridin-5-yl]-3-methyl-5-(trifluoromethyl)phenol;
5-chloro-2-[2-(5,7-dihydropyrrolo[3,4-d]pyrimidin-6-yl)oxazolo[4,5-b]pyridin-5-yl]-3-methyl-phenol; formic acid;
5-chloro-2-[2-(5,7-dihydropyrrolo[3,4-d]pyrimidin-6-yl)oxazolo[4,5-b]pyridin-5-yl]-3-methyl-phenol;
5-chloro-2-[2-(4,6-dihydro-1H-pyrrolo[3,4-c]pyrazol-5-yl)oxazolo[4,5-b]pyridin-5-yl]-3-methyl-phenol;
5-chloro-2-[2-(3,4-dihydro-1H-2,7-naphthyridin-2-yl)oxazolo[4,5-b]pyridin-5-yl]-3-methyl-phenol;
3-hydroxy-4-(2-isoindolin-2-yloxazolo[4,5-b]pyridin-5-yl)-5-methyl-benzonitrile;
4-[2-(7,8-dihydro-5H-pyrido[4,3-d]pyrimidin-6-yl)oxazolo[4,5-b]pyridin-5-yl]-3-hydroxy-5-methyl-benzonitrile;
2-[2-(5,7-dihydropyrrolo[3,4-d]pyrimidin-6-yl)oxazolo[4,5-b]pyridin-5-yl]-3-methyl-5-(trifluoromethyl)phenol;
7-[5-[2-hydroxy-6-methyl-4-(trifluoromethyl)phenyl]oxazolo[4,5-b]pyridin-2-yl]-2,5,6,8-tetrahydro-2,7-naphthyridin-3-one;
6-[5-[2-hydroxy-6-methyl-4-(trifluoromethyl)phenyl]oxazolo[4,5-b]pyridin-2-yl]-5,7-dihydro-1H-pyrrolo[3,4-b]pyridin-2-one;
6-[5-[2-hydroxy-6-methyl-4-(trifluoromethyl)phenyl]oxazolo[4,5-b]pyridin-2-yl]-1-methyl-5,7-dihydropyrrolo[3,4-b]pyridin-2-one;
6-[5-[2-hydroxy-6-methyl-4-(trifluoromethyl)phenyl]oxazolo[4,5-b]pyridin-2-yl]-2-methyl-5,7-dihydropyrrolo[3,4-c]pyridazin-3-one;
2-[2-(5,7-dihydropyrrolo[3,4-b]pyrazin-6-yl)oxazolo[4,5-b]pyridin-5-yl]-3-methyl-5-(trifluoromethyl)phenol;
2-[2-(7,8-dihydro-5H-pyrido[4,3-d]pyrimidin-6-yl)oxazolo[4,5-b]pyridin-5-yl]-3-methyl-5-(trifluoromethyl)phenol;
5-chloro-3-methyl-2-[2-(2,4,6,7-tetrahydropyrazolo[4,3-c]pyridin-5-yl)oxazolo[4,5-b]pyridin-5-yl]phenol;
5-(difluoromethyl)-2-[2-(7,8-dihydro-5H-pyrido[4,3-d]pyrimidin-6-yl)oxazolo[4,5-b]pyridin-5-yl]-3-methyl-phenol;
7-[5-(4-chloro-2-hydroxy-6-methyl-phenyl)oxazolo[4,5-b]pyridin-2-yl]-2,5,6,8-tetrahydro-2,7-naphthyridin-3-one;
5-Chloro-2-[2-(1,3-dihydropyrrolo[3,4-c]pyridin-2-yl)oxazolo[4,5-b]pyrazin-5-yl]-3-methyl-phenol; formic acid;
5-Chloro-2-[2-(1,3-dihydropyrrolo[3,4-c]pyridin-2-yl)oxazolo[4,5-b]pyrazin-5-yl]-3-methyl-phenol;
5-Chloro-2-[2-(1,3-dihydropyrrolo[3,4-c]pyridin-2-yl)oxazolo[4,5-b]pyrazin-5-yl]-3-(hydroxymethyl)phenol;
5-Chloro-2-[2-(7,8-dihydro-5H-pyrido[4,3-d]pyrimidin-6-yl)oxazolo[4,5-b]pyridin-5-yl]-3-methyl-phenol;
5-Chloro-2-[2-(2-methoxy-7,8-dihydro-5H-pyrido[4,3-d]pyrimidin-6-yl)oxazolo[4,5-b]pyridin-5-yl]-3-methyl-phenol;
2-[5-(4-Chloro-2-hydroxy-6-methyl-phenyl)oxazolo[4,5-b]pyridin-2-yl]-3,4-dihydro-1H-isoquinoline-5-carbonitrile;
5-Chloro-2-[2-(5-methoxy-3,4-dihydro-1H-isoquinolin-2-yl)oxazolo[4,5-b]pyridin-5-yl]-3-methyl-phenol;
5-Chloro-2-[2-(7-fluoro-3,4-dihydro-1H-isoquinolin-2-yl)oxazolo[4,5-b]pyridin-5-yl]-3-methyl-phenol;
6-[5-(4-Chloro-2-hydroxy-6-methyl-phenyl)oxazolo[4,5-b]pyridin-2-yl]-1,5,7,8-tetrahydropyrido[4,3-d]pyrimidin-2-one;
6-[5-(4-Chloro-2-hydroxy-6-methyl-phenyl)oxazolo[4,5-b]pyridin-2-yl]-1,5,7,8-tetrahydro-1,6-naphthyridin-2-one;
6-[5-(4-Chloro-2-hydroxy-6-methyl-phenyl)oxazolo[4,5-b]pyridin-2-yl]-5,7-dihydro-2H-pyrrolo[3,4-c]pyridazin-3-one;
5-chloro-2-[2-(7,8-dihydro-5H-pyrido[3,4-b]pyrazin-6-yl)oxazolo[4,5-b]pyridin-5-yl]-3-methyl-phenol;
6-[5-[2-hydroxy-6-methyl-4-(trifluoromethyl)phenyl]oxazolo[4,5-b]pyridin-2-yl]-3,5,7,8-tetrahydropyrido[4,3-d]pyrimidin-4-one;
5-chloro-2-(2-(6,8-dihydro-7H-[1,3]dioxolo[4,5-e]isoindol-7-yl)oxazolo[4,5-b]pyridin-5-yl)-3-methylphenol; and
pharmaceutically acceptable salts thereof.

24-27. (canceled)

28. A method of treating a disease, disorder or condition responsive to NLRP3 inhibition in a subject in need thereof, comprising administering a therapeutically effective amount of a compound according to claim 1.

29. A pharmaceutical composition comprising a compound according to claim 1 and a therapeutically inert carrier.

30. (canceled)

31. The method of claim 28, wherein the disease, disorder or condition is selected from Asthma and chronic obstructive pulmonary disorder (COPD).

32. The method of claim 28, wherein the disease, disorder or condition is selected from Parkinson's Disease and Alzheimer's Disease.

33-34. (canceled)

35. A method of inhibiting NLRP3 in a subject in need thereof, which method comprises administering to the subject an effective amount of a compound of claim 1.

36-38. (canceled)
